SEQUENCE LISTING (317 sequences)

SEQ ID NO: 1 - protein, 167 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 2 - protein, 167 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARGAKTGAAGSLMDVLHHPGMNHRVEITEGILADECAALLSDFFRMRRQEIKAQKKAQSSTD

SEQ ID NO: 3 - protein, 167 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARGAKTGAAGSLMDVLHHPGMNHRVEITEGILADECAALLSDFFRMRRQEIKAQKNAQSSTD

SEQ ID NO: 4 - protein, 167 aa, synthetic construct
MSEVEFSHEYWMRHALTLAKRAWDEREVPVGAVLVHNNRVIGEGWNRAIGRHDPTAHAEIMALRQGGLVMQNYRLIDATLYVTLEPCVMCAGAMIHSRIGRVVFGARGAKTGAAGSLMDVLHHPGMNHRVEITEGILADECAALLSDFFRMRRQEIKAQKNAQSSTD

SEQ ID NO: 5 - protein, 167 aa, synthetic construct
MSEVEFSHEYWMRHALTLAKRAWDEREVPVGAVLVHNNRVIGEGWNRAIGRHDPTAHAEIMALRQGGLVMQNYRLFDATLYVTLEPCVMCAGAMIHSRIGRVVFGARGAKTGAAGSLMDVLHHPGMNHRVEITEGILADECAALLSDFFRMRRQEIKAQKNAQSSTD

SEQ ID NO: 6 - protein, 167 aa, synthetic construct
MSEVEFSHEYWMRHALTLAKRAWDEREVPVGAVLVHNNRVIGEGWNRAIGRHDPTAHAEIMALRQGGLVMQNYRLIDATLYVTLEPCVMCAGAMIHSRIGRVVFGARGAKTGAAGSLMDVLHHPGINHRVEITEGILADECAALLSDFFRMRRQEIKAQKNAQSSTD

SEQ ID NO: 7 - protein, 167 aa, synthetic construct
MSEVEFSHEYWMRHALTLAKRAWDEREVPVGAVLVHNNRVIGEGWNRAIGRHDPTAHAEIMALRQGGLVMQNYRLIDATLYVTLEPCVMCAGAMIHSRIGRVVFGARGAKTGAAGSLMDVLHHPGMKHRVEITEGILADECAALLSDFFRMRRQEIKAQKNAQSSTD

SEQ ID NO: 8 - protein, 167 aa, synthetic construct
MSEVEFSHEYWMRHALTLAKRAWDEREVPVGAVLVHNNRVIGEGWNRAIGRHDPTAHAEIMALRQGGLVMQNYRLFDATLYVTLEPCVMCAGAMIHSRIGRVVFGARGARTGAAGSLMDVLHHPGMKHRVEITEGILADECAALLSDFFRMRRQEIKAQKNAQSSTD

SEQ ID NO: 9 - protein, 167 aa, synthetic construct
MSEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGARGARTGAAGSLMDVLHHPGIKHRVEITEGILADECAALLSDFFRMRRQEIKAQKNAQSSTD

SEQ ID NO: 10 - protein, 167 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARGARTGAAGSLMDVLHHPGMKHRVEITEGILADECAALLSDFFRMRRQEIKAQKNAQSSTD

SEQ ID NO: 11 - protein, 167 aa, synthetic construct
MSEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLSRFFRMPRRVFKAQKKAQSSTD

SEQ ID NO: 12 - protein, 167 aa, synthetic construct
MSEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLCRFFRMPRRVFKAQKKAQSSTD

SEQ ID NO: 13 - protein, 167 aa, synthetic construct
MSEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLSRFFRMPRRVFNAQKKAQSSTD

SEQ ID NO: 14 - protein, 167 aa, synthetic construct
MSEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLSRFFRMPRRVFKAQKNAQSSTD

SEQ ID NO: 15 - protein, 167 aa, synthetic construct
MSEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLSRFFRMPRRVFKAQKKAQSSID

SEQ ID NO: 16 - protein, 167 aa, synthetic construct
MSEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGARGARTGAAGSLMDVLRHPGIKHRVEITEGILADECAALLSDFFRMRRQEIKAQKNAQSSTD

SEQ ID NO: 17 - protein, 167 aa, synthetic construct
MSEVEFSHEYWMRHALTLAKRAWDEREVPVGAVLVHNNRVIGEGWNRAIGHHDPTAHAEIMALRQGGLVMQNYRLIDATLYVTLEPCVMCAGAMIHSRIGRVVFGARGARTGAAGSLMDVLRHPGIKHRVEITEGILADECAALLSDFFRMRRQEIKAQKKAQSSTD

SEQ ID NO: 18 - protein, 167 aa, synthetic construct
MSEVEFSHEYWMRHALTLAKRAWDERDVPVGAVLVHNNRVIGEGWNRAIGHHDPTAHAEIMALRQGGLVMQNYRLFDATLYVTLEPCVMCAGAMIHSRIGRVVFGARGARTGAAGSLMDVLRHPGIKHRVEITEGILADECAALLSDFFRMRRQEIKAQKNAQSSTD

SEQ ID NO: 19 - protein, 167 aa, synthetic construct
MSEVEFSHEYWMRHALTLAKRAWDERDVPVGAVLVHNNRVIGEGWNKAIGHHDPTAHAEIMALRQGGLVMQNYRLFDATLYVTLEPCVMCAGAMIHSRIGRVVFGARGARTGAAGSLMDVLRHPGIKHRVEITEGILADECAALLSDFFRMRRQEIKAQKNAQSSTD

SEQ ID NO: 20 - protein, 167 aa, synthetic construct
MSEVEFSHEYWMRHALTLAKRAWDERDVPVGAVLVHNNRVIGEGWNRAIGHHDPTAHAEIMALRQGGLVMQNYRLIDATLYVTLEPCVMCAGAMIHSRIGRVVFGARGARTGAVGSLMDVLRHPGIKHRVEITEGILADECAALLSDFFRMRRQEIKAQKKAQSSTD

SEQ ID NO: 21 - protein, 167 aa, synthetic construct
MSEVEFSHEYWMRHALTLAKRAWDERDVPVGAVLVHNNRVIGEGWNKAIGHHDPTAHAEIMALRQGGLVMQNYRLFDATLYVTLEPCVMCAGAMIHSRIGRVVFGARGARTGAVGSLMDVLRHPGIKHRVEITEGILADECAALLSDFFRMRRQEIKAQKKAQSSTD

SEQ ID NO: 22 - protein, 167 aa, synthetic construct
MSEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLRYPGIKHRVEITEGILADECAALLSRFFRMPRRVFKAQKKAQSSTD

SEQ ID NO: 23 - protein, 167 aa, synthetic construct
MSEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLSRFFRMPRRVFKAQKKAQSSTN

SEQ ID NO: 24 - protein, 167 aa, synthetic construct
MSEVEFSHEYWMRHALTLAKRARDEREVPVGAVLVLNNRVIGEGWNKAIGLHDPTAHAEIMALRQGGLVMQNYRLFDATLYSTLEPCVMCAGAMIHSRIGRVVFGVRGARHGAAGSLMNVLHYPGIKHRVEITEGILADECAALLCRFFRMPRRVFKAQKKAQSSTD

SEQ ID NO: 25 - protein, 167 aa, synthetic construct
MSEVEFSHEYWMRHALTLAKRARDEREVPVGAVLVLNNRVIGEGWNKAIGLHDPTAHAEIMALRQGGLVMQNYRLFDATLYSTLEPCVMCAGAMIHSRIGRVVFGVRGARHGAAGSLMNVLNYPGIKHRVEITEGILADECAALLCRFFRMPRRVFKAQKKAQSSTD

SEQ ID NO: 26 - protein, 167 aa, synthetic construct
MSEVEFSHEYWMRHALTLAKRARDEREVPVGAVLVLNNRVIGEGWNKAIGLHDPTAHAEIMALRQGGLVMQNYRLYDATLYSTLEPCVMCAGAMIHSRIGRVVFGVRGARHGAVGSLMNVLHYPGIKHRVEITEGILADECAALLCRFFRMPRRVFKAQKKAQSSTD

SEQ ID NO: 27 - protein, 167 aa, synthetic construct
MSEVEFSHEYWMRHALTLAKRARDEREVPVGAVLVLNNRVIGEGWNKAIGLHDPTAHAEIMALRQGGLVMQNYRLFDATLYSTLEPCVMCAGAMIHSRIGRVVFGVRGSRHGAVGSLMNVLHYPGIKHRVEITEGILADECAALLSRFFRMPRRVFKAQKKAQSSTD

SEQ ID NO: 28 - protein, 167 aa, synthetic construct
MSEVEFSHEYWMRHALTLAKRARDEREVPVGAVLVHNNRVIGEGWNKAIGLHDPTAHAEIMALRQGGLVMQNYRLYDATLYSTLEPCVMCAGAMIHSRIGRVVFGVRGSRHGAVGSLMNVLHYPGIKHRVEITEGILADECAALLSRFFRMPRRVFKAQKKAQSSTD

SEQ ID NO: 29 - protein, 167 aa, synthetic construct
MSEVEFSHEYWMRHALTLAKRARDEREVPVGAVLVLNNRVIGEGWNKAIGLHDPTAHAEIMALRQGGLVMQNYRLYDATLYSTLEPCVMCAGAMIHSRIGRVVFGVRGARHGAVGSLMNVLHYPGIKHRVEITEGILADECAALLCRFFRMPRRVFNAQKNAQSSIN

SEQ ID NO: 30 - protein, 167 aa, synthetic construct
MSEVEFSHEYWMRHALTLAKRARDEREVPVGAVLVLNNRVIGEGWNKAIGLHDPTAHAEIMALRQGGLVMQNYRLYDATLYSTLEPCVMCAGAMIHSRIGRVVFGVRGARHGAVGSLMNVLHYPGIKHRVEITEGILADECAALLCRFFRMPRRVFKAQKKAQSSIN

SEQ ID NO: 31 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 32 - protein, 167 aa, synthetic construct
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 33 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 34 - DNA, 29 bp, synthetic construct
  source 1..29: mol_type = other DNA
  modified_base 6: mod_base = OTHER; note = uracil
agagctagaaatagcaagttaaaataagg

SEQ ID NO: 35 - DNA, 53 bp, synthetic construct
  source 1..53: mol_type = other DNA
  modified_base 6: mod_base = OTHER; note = uracil
agctctaaaacgcagtctatgctttgtgttcggtgtttcgtcctttccacaag

SEQ ID NO: 36 - DNA, 54 bp, synthetic construct
  source 1..54: mol_type = other DNA
  modified_base 6: mod_base = OTHER; note = uracil
agctctaaaacccacccaagtgatcacacttcggtgtttcgtcctttccacaag

SEQ ID NO: 37 - DNA, 54 bp, synthetic construct
  source 1..54: mol_type = other DNA
  modified_base 6: mod_base = OTHER; note = uracil
agctctaaaacccccaaaggtgaccgtcctgcggtgtttcgtcctttccacaag

SEQ ID NO: 38 - DNA, 54 bp, synthetic construct
  source 1..54: mol_type = other DNA
  modified_base 6: mod_base = OTHER; note = uracil
agctctaaaacccaagacaaacttgcatcctcggtgtttcgtcctttccacaag

SEQ ID NO: 39 - DNA, 53 bp, synthetic construct
  source 1..53: mol_type = other DNA
  modified_base 6: mod_base = OTHER; note = uracil
agctctaaaaccctgacaatcgataggtaccggtgtttcgtcctttccacaag

SEQ ID NO: 40 - DNA, 53 bp, synthetic construct
  source 1..53: mol_type = other DNA
  modified_base 6: mod_base = OTHER; note = uracil
agctctaaaacgcagtctatgcctcatactcggtgtttcgtcctttccacaag

SEQ ID NO: 41 - DNA, 53 bp, synthetic construct
  source 1..53: mol_type = other DNA
  modified_base 6: mod_base = OTHER; note = uracil
agctctaaaacgccctggcctgggtcaatccggtgtttcgtcctttccacaag

SEQ ID NO: 42 - DNA, 53 bp, synthetic construct
  source 1..53: mol_type = other DNA
  modified_base 6: mod_base = OTHER; note = uracil
agctctaaaacgcagtctatccttggtcttcggtgtttcgtcctttccacaag

SEQ ID NO: 43 - DNA, 54 bp, synthetic construct
  source 1..54: mol_type = other DNA
  modified_base 6: mod_base = OTHER; note = uracil
agctctaaaaccaaaggtgaccgtcctggctcggtgtttcgtcctttccacaag

SEQ ID NO: 44 - DNA, 53 bp, synthetic construct
  source 1..53: mol_type = other DNA
  modified_base 6: mod_base = OTHER; note = uracil
agctctaaaaccccaagtgatcacacttgtcggtgtttcgtcctttccacaag

SEQ ID NO: 45 - DNA, 54 bp, synthetic construct
  source 1..54: mol_type = other DNA
  modified_base 6: mod_base = OTHER; note = uracil
agctctaaaactggcctgggtcaatccttggcggtgtttcgtcctttccacaag

SEQ ID NO: 46 - DNA, 54 bp, synthetic construct
  source 1..54: mol_type = other DNA
  modified_base 6: mod_base = OTHER; note = uracil
agctctaaaaccagctacctgaagtacttggcggtgtttcgtcctttccacaag

SEQ ID NO: 47 - DNA, 53 bp, synthetic construct
  source 1..53: mol_type = other DNA
  modified_base 6: mod_base = OTHER; note = uracil
agctctaaaactgactcatcattatctcatcggtgtttcgtcctttccacaag

SEQ ID NO: 48 - DNA, 54 bp, synthetic construct
  source 1..54: mol_type = other DNA
  modified_base 6: mod_base = OTHER; note = uracil
agctctaaaactttaatcataacaattgcttcggtgtttcgtcctttccacaag

SEQ ID NO: 49 - DNA, 54 bp, synthetic construct
  source 1..54: mol_type = other DNA
  modified_base 6: mod_base = OTHER; note = uracil
agctctaaaaccatttcttttggaatgtattcggtgtttcgtcctttccacaag

SEQ ID NO: 50 - DNA, 53 bp, synthetic construct
  source 1..53: mol_type = other DNA
  modified_base 6: mod_base = OTHER; note = uracil
agctctaaaacatttcttttggaatgtattcggtgtttcgtcctttccacaag

SEQ ID NO: 51 - DNA, 54 bp, synthetic construct
  source 1..54: mol_type = other DNA
  modified_base 6: mod_base = OTHER; note = uracil
agctctaaaactttcttttggaatgtattcacggtgtttcgtcctttccacaag

SEQ ID NO: 52 - DNA, 54 bp, synthetic construct
  source 1..54: mol_type = other DNA
  modified_base 6: mod_base = OTHER; note = uracil
agctctaaaaccactatctcaatgcaaatatcggtgtttcgtcctttccacaag

SEQ ID NO: 53 - DNA, 54 bp, synthetic construct
  source 1..54: mol_type = other DNA
  modified_base 6: mod_base = OTHER; note = uracil
agctctaaaacgcaccttggcgcagcggtggcggtgtttcgtcctttccacaag

SEQ ID NO: 54 - DNA, 54 bp, synthetic construct
  source 1..54: mol_type = other DNA
  modified_base 6: mod_base = OTHER; note = uracil
agctctaaaacgcttgcccccttgggccttacggtgtttcgtcctttccacaag

SEQ ID NO: 55 - DNA, 53 bp, synthetic construct
  source 1..53: mol_type = other DNA
  modified_base 6: mod_base = OTHER; note = uracil
agctctaaaaccgcaggccacggtcacctgcggtgtttcgtcctttccacaag

SEQ ID NO: 56 - DNA, 53 bp, synthetic construct
  source 1..53: mol_type = other DNA
  modified_base 6: mod_base = OTHER; note = uracil
agctctaaaactcacgtgctcagtctgggccggtgtttcgtcctttccacaag

SEQ ID NO: 57 - DNA, 53 bp, synthetic construct
  source 1..53: mol_type = other DNA
  modified_base 6: mod_base = OTHER; note = uracil
agctctaaaacttcttcttctgctcggactcggtgtttcgtcctttccacaag

SEQ ID NO: 58 - DNA, 34 bp, synthetic construct
  source 1..34: mol_type = other DNA
  modified_base 8: mod_base = OTHER; note = uracil
agtactctggaaacagaatctactaaaacaaggc

SEQ ID NO: 59 - DNA, 57 bp, synthetic construct
  source 1..57: mol_type = other DNA
  modified_base 8: mod_base = OTHER; note = uracil
agagtactaaaactaggacacatgctgtctaccacggtgtttcgtcctttccacaag

SEQ ID NO: 60 - DNA, 58 bp, synthetic construct
  source 1..58: mol_type = other DNA
  modified_base 8: mod_base = OTHER; note = uracil
agagtactaaaacccccaaaggccaggctgtaaatcggtgtttcgtcctttccacaag

SEQ ID NO: 61 - DNA, 57 bp, synthetic construct
  source 1..57: mol_type = other DNA
  modified_base 8: mod_base = OTHER; note = uracil
agagtactaaaactgtttagcacattacctgacacggtgtttcgtcctttccacaag

SEQ ID NO: 62 - DNA, 57 bp, synthetic construct
  source 1..57: mol_type = other DNA
  modified_base 8: mod_base = OTHER; note = uracil
agagtactaaaacaccccatgcaccctcctccaccggtgtttcgtcctttccacaag

SEQ ID NO: 63 - DNA, 57 bp, synthetic construct
  source 1..57: mol_type = other DNA
  modified_base 8: mod_base = OTHER; note = uracil
agagtactaaaactggctcaatcaatcctcttgccggtgtttcgtcctttccacaag

SEQ ID NO: 64 - DNA, 58 bp, synthetic construct
  source 1..58: mol_type = other DNA
  modified_base 8: mod_base = OTHER; note = uracil
agagtactaaaacttatgatacttcgcacactagtcggtgtttcgtcctttccacaag

SEQ ID NO: 65 - DNA, 53 bp, synthetic construct
  source 1..53: mol_type = other DNA
  modified_base 6: mod_base = OTHER; note = uracil
agctctaaaacggagtttggccttgttaaccggtgtttcgtcctttccacaag

SEQ ID NO: 66 - DNA, 53 bp, synthetic construct
  source 1..53: mol_type = other DNA
  modified_base 6: mod_base = OTHER; note = uracil
agctctaaaacctaatcccggaactggacccggtgtttcgtcctttccacaag

SEQ ID NO: 67 - DNA, 54 bp, synthetic construct
  source 1..54: mol_type = other DNA
  modified_base 6: mod_base = OTHER; note = uracil
agctctaaaacagcccagcagtctatccttgcggtgtttcgtcctttccacaag

SEQ ID NO: 68 - DNA, 53 bp, synthetic construct
  source 1..53: mol_type = other DNA
  modified_base 6: mod_base = OTHER; note = uracil
agctctaaaacgccgtttgtactttgtcctcggtgtttcgtcctttccacaag

SEQ ID NO: 69 - DNA, 53 bp, synthetic construct
  source 1..53: mol_type = other DNA
  modified_base 6: mod_base = OTHER; note = uracil
agctctaaaacgccagataatacgggtcatcggtgtttcgtcctttccacaag

SEQ ID NO: 70 - DNA, 53 bp, synthetic construct
  source 1..53: mol_type = other DNA
  modified_base 6: mod_base = OTHER; note = uracil
agctctaaaacagtcatggtttgatgtctccggtgtttcgtcctttccacaag

SEQ ID NO: 71 - DNA, 54 bp, synthetic construct
  source 1..54: mol_type = other DNA
  modified_base 6: mod_base = OTHER; note = uracil
agctctaaaacgtgacaagtgtgatcacttgcggtgtttcgtcctttccacaag

SEQ ID NO: 72 - DNA, 54 bp, synthetic construct
  source 1..54: mol_type = other DNA
  modified_base 6: mod_base = OTHER; note = uracil
agctctaaaactgatgtctcctgcagtctatcggtgtttcgtcctttccacaag

SEQ ID NO: 73 - DNA, 54 bp, synthetic construct
  source 1..54: mol_type = other DNA
  modified_base 6: mod_base = OTHER; note = uracil
agctctaaaaccttcttcatctgcaagtcatcggtgtttcgtcctttccacaag

SEQ ID NO: 74 - DNA, 53 bp, synthetic construct
  source 1..53: mol_type = other DNA
  modified_base 6: mod_base = OTHER; note = uracil
agctctaaaactggaaaaatggctttgaatcggtgtttcgtcctttccacaag

SEQ ID NO: 75 - DNA, 58 bp, synthetic construct
  source 1..58: mol_type = other DNA
  modified_base 8: mod_base = OTHER; note = uracil
agagtactaaaacgtcaggcctctgtccctctgtacggtgtttcgtcctttccacaag

SEQ ID NO: 76 - DNA, 58 bp, synthetic construct
  source 1..58: mol_type = other DNA
  modified_base 8: mod_base = OTHER; note = uracil
agagtactaaaacaggctgttgtcatacttctcatcggtgtttcgtcctttccacaag

SEQ ID NO: 77 - DNA, 57 bp, synthetic construct
  source 1..57: mol_type = other DNA
  modified_base 8: mod_base = OTHER; note = uracil
agagtactaaaacggtaatgactaagatgactgccggtgtttcgtcctttccacaag

SEQ ID NO: 78 - DNA, 57 bp, synthetic construct
  source 1..57: mol_type = other DNA
  modified_base 8: mod_base = OTHER; note = uracil
agagtactaaaacgggtacaatcctactctagtccggtgtttcgtcctttccacaag

SEQ ID NO: 79 - DNA, 57 bp, synthetic construct
  source 1..57: mol_type = other DNA
  modified_base 8: mod_base = OTHER; note = uracil
agagtactaaaactgctgtcacagttagctcagccggtgtttcgtcctttccacaag

SEQ ID NO: 80 - DNA, 43 bp, synthetic construct
  source 1..43: mol_type = other DNA
  modified_base 9: mod_base = OTHER; note = uracil
atctacacttagtagaaattcggtgtttcgtcctttccacaag

SEQ ID NO: 81 - DNA, 57 bp, synthetic construct
  source 1..57: mol_type = other DNA
  modified_base 9: mod_base = OTHER; note = uracil
agtgtagattgctgcaagtaagcatgcatttgtttttttaagcttgggccgctcgag

SEQ ID NO: 82 - DNA, 57 bp, synthetic construct
  source 1..57: mol_type = other DNA
  modified_base 9: mod_base = OTHER; note = uracil
agtgtagatctagacaggggctagtatgtgcatttttttaagcttgggccgctcgag

SEQ ID NO: 83 - DNA, 57 bp, synthetic construct
  source 1..57: mol_type = other DNA
  modified_base 9: mod_base = OTHER; note = uracil
agtgtagatcagctattcaggctggcccgccctttttttaagcttgggccgctcgag

SEQ ID NO: 84 - DNA, 57 bp, synthetic construct
  source 1..57: mol_type = other DNA
  modified_base 9: mod_base = OTHER; note = uracil
agtgtagatgaagcacatcaaggacattctaatttttttaagcttgggccgctcgag

SEQ ID NO: 85 - DNA, 57 bp, synthetic construct
  source 1..57: mol_type = other DNA
  modified_base 9: mod_base = OTHER; note = uracil
agtgtagatggataagcacagttttaaatagttttttttaagcttgggccgctcgag

SEQ ID NO: 86 - DNA, 57 bp, synthetic construct
  source 1..57: mol_type = other DNA
  modified_base 9: mod_base = OTHER; note = uracil
agtgtagatgtttaaacacaccgggttaataatttttttaagcttgggccgctcgag

SEQ ID NO: 87 - DNA, 38 bp, synthetic construct
  source 1..38: mol_type = other DNA
  modified_base 8: mod_base = OTHER; note = uracil
acaagagtagaaattcggtgtttcgtcctttccacaag

SEQ ID NO: 88 - DNA, 60 bp, synthetic construct
  source 1..60: mol_type = other DNA
  modified_base 8: mod_base = OTHER; note = uracil
actcttgtagattgctgcaagtaagcatgcatttgtttttttaagcttgggccgctcgag

SEQ ID NO: 89 - DNA, 60 bp, synthetic construct
  source 1..60: mol_type = other DNA
  modified_base 8: mod_base = OTHER; note = uracil
actcttgtagatctagacaggggctagtatgtgcatttttttaagcttgggccgctcgag

SEQ ID NO: 90 - DNA, 60 bp, synthetic construct
  source 1..60: mol_type = other DNA
  modified_base 8: mod_base = OTHER; note = uracil
actcttgtagatcagctattcaggctggcccgccctttttttaagcttgggccgctcgag

SEQ ID NO: 91 - DNA, 60 bp, synthetic construct
  source 1..60: mol_type = other DNA
  modified_base 8: mod_base = OTHER; note = uracil
actcttgtagatgaagcacatcaaggacattctaatttttttaagcttgggccgctcgag

SEQ ID NO: 92 - DNA, 60 bp, synthetic construct
  source 1..60: mol_type = other DNA
  modified_base 8: mod_base = OTHER; note = uracil
actcttgtagatggataagcacagttttaaatagttttttttaagcttgggccgctcgag

SEQ ID NO: 93 - DNA, 60 bp, synthetic construct
  source 1..60: mol_type = other DNA
  modified_base 8: mod_base = OTHER; note = uracil
actcttgtagatgtttaaacacaccgggttaataatttttttaagcttgggccgctcgag

SEQ ID NO: 94 - DNA, 54 bp, synthetic construct
  source 1..54: mol_type = other DNA
  modified_base 6: mod_base = OTHER; note = uracil
agctctaaaacctccagggcgaacttcgacacggtgtttcgtcctttccacaag

SEQ ID NO: 95 - DNA, 54 bp, synthetic construct
  source 1..54: mol_type = other DNA
  modified_base 6: mod_base = OTHER; note = uracil
agctctaaaaccctctccagggcgaacttcgcggtgtttcgtcctttccacaag

SEQ ID NO: 96 - DNA, 20 bp, synthetic construct
  source 1..20: mol_type = other DNA
gctctgatctgaataccacg

SEQ ID NO: 97 - DNA, 20 bp, synthetic construct
  source 1..20: mol_type = other DNA
gcttgatcggagaggctatt

SEQ ID NO: 98 - DNA, 20 bp, synthetic construct
  source 1..20: mol_type = other DNA
ttcttttcagtgccattggg

SEQ ID NO: 99 - DNA, 20 bp, synthetic construct
  source 1..20: mol_type = other DNA
gactgatcgcaacagacaat

SEQ ID NO: 100 - DNA, 20 bp, synthetic construct
  source 1..20: mol_type = other DNA
gtcaggctgcaatgtgaata

SEQ ID NO: 101 - DNA, 20 bp, synthetic construct
  source 1..20: mol_type = other DNA
tacggcgtagtgcacctgga

SEQ ID NO: 102 - DNA, 44 bp, synthetic construct
  source 1..44: mol_type = other DNA
gattggtctcaacctgcaggtgcagtaaggaggaaaaaaaaatg

SEQ ID NO: 103 - DNA, 34 bp, synthetic construct
  source 1..34: mol_type = other DNA
gattggtctcagtccccggtgtttcgctaccgga

SEQ ID NO: 104 - DNA, 37 bp, synthetic construct
  source 1..37: mol_type = other DNA
ccaccctgtatgtgacattcgagccatgcgtgatgtg

SEQ ID NO: 105 - DNA, 37 bp, synthetic construct
  source 1..37: mol_type = other DNA
ccaccctgtatgtgacactggagccatgcgtgatgtg

SEQ ID NO: 106 - DNA, 43 bp, synthetic construct
  source 1..43: mol_type = other DNA
tgtcacatacagggtggcatcgawcaggcggtaattctgcatg

SEQ ID NO: 107 - DNA, 37 bp, synthetic construct
  source 1..37: mol_type = other DNA
cgtctgccaggattccctctgtgatctccacccggtg

SEQ ID NO: 108 - DNA, 33 bp, synthetic construct
  source 1..33: mol_type = other DNA
ggaatcctggcagacgagtgcgccgccctgctg

SEQ ID NO: 109 - DNA, 148 bp, synthetic construct
  source 1..148: mol_type = other DNA
gagccatgcgtgatgtgcgcaggagcaatgatccacagcaggatcggaagagtggtgttcggagygcggggcgccargcgcggcgcagcaggctccctgatgratgtgctgcrcyaccccggcatraascaccgggtggagatcacag

SEQ ID NO: 110 - DNA, 148 bp, synthetic construct
  source 1..148: mol_type = other DNA
gagccatgcgtgatgtgcgcaggagcaatgatccacagcaggatcggaagagtggtgttcggagygcggggcgccargaccggcgcagcaggctccctgatgratgtgctgcrcyaccccggcatraascaccgggtggagatcacag

SEQ ID NO: 111 - DNA, 100 bp, synthetic construct
  source 1..100: mol_type = other DNA
gtgcgccgccctgctgwgccgtttctwtagaatgcsgagacrggwgwtcaakgcccagaagaasgcacagagctccaycractccggtagcgaaacaccg

SEQ ID NO: 112 - DNA, 100 bp, synthetic construct
  source 1..100: mol_type = other DNA
gtgcgccgccctgctgwgcgatttctwtagaatgcsgagacrggwgwtcaakgcccagaagaasgcacagagctccaycractccggtagcgaaacaccg

SEQ ID NO: 113 - DNA, 42 bp, synthetic construct
  source 1..42: mol_type = other DNA
ggcgcccacggggacwtctctttcatcccrtgctcgctttgc

SEQ ID NO: 114 - DNA, 48 bp, synthetic construct
  source 1..48: mol_type = other DNA
ccccgtgggcgccgtgctggtgcwcaacaatagagtgatcggagaggg

SEQ ID NO: 115 - DNA, 53 bp, synthetic construct
  source 1..53: mol_type = other DNA
gcggtagggtcgtggwggccgattgscytgttccatccctctccgatcactct

SEQ ID NO: 116 - DNA, 19 bp, synthetic construct
  source 1..19: mol_type = other DNA
cacgaccctaccgcacacg

SEQ ID NO: 117 - DNA, 60 bp, synthetic construct
  source 1..60: mol_type = other DNA
acatcacgcatggctcgartgtcacatacagggtggcatcgwacaggcggtaattctgca

SEQ ID NO: 118 - DNA, 60 bp, synthetic construct
  source 1..60: mol_type = other DNA
acatcacgcatggctcgartgtcgaatacagggtggcatcgwacaggcggtaattctgca

SEQ ID NO: 119 - DNA, 60 bp, synthetic construct
  source 1..60: mol_type = other DNA
gccatgcgtgatgtgcgcaggagcartgatccacagcaggatcggaagagtggtgttcgg

SEQ ID NO: 120 - DNA, 60 bp, synthetic construct
  source 1..60: mol_type = other DNA
cattcatcagggagcctrctgcgccgygcctggmgccccgcactccgaacaccactcttc

SEQ ID NO: 121 - DNA, 60 bp, synthetic construct
  source 1..60: mol_type = other DNA
cattcatcagggagcctrctgcgccgygcctggmgttccgcactccgaacaccactcttc

SEQ ID NO: 122 - DNA, 33 bp, synthetic construct
  source 1..33: mol_type = other DNA
ggctccctgatgaatgtgctgmactaccccggc

SEQ ID NO: 123 - DNA, 19 bp, synthetic construct
  source 1..19: mol_type = other DNA
cattttgcgcttcagccat

SEQ ID NO: 124 - DNA, 19 bp, synthetic construct
  source 1..19: mol_type = other DNA
cagtgatcaccgcccatcc

SEQ ID NO: 125 - RNA, 77 nt, synthetic construct
  source 1..77: mol_type = other RNA
gcatccgtagctcagctggatagagtactcggctacgaaccgagcggtcggaggttcgaatcctcccggatgcacca

SEQ ID NO: 126 - DNA, 58 bp, synthetic construct
  source 1..58: mol_type = other DNA
tccgaatagcgcccttccccttgcccggcgttaatgatttgcccaaatggtgcatccg

SEQ ID NO: 127 - DNA, 56 bp, synthetic construct
  source 1..56: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnngcatccgtagctcagctgg

SEQ ID NO: 128 - DNA, 50 bp, synthetic construct
  source 1..50: mol_type = other DNA
gactggagttcagacgtgtgctcttccgatcttccgaatagcgcccttcc

SEQ ID NO: 129 - DNA, 22 bp, synthetic construct
  source 1..22: mol_type = other DNA
tgggttggtgatcgtttggtgg

SEQ ID NO: 130 - DNA, 22 bp, synthetic construct
  source 1..22: mol_type = other DNA
tgggttggttatcgtttggtgg

SEQ ID NO: 131 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnccagccccatctgtcaaact

SEQ ID NO: 132 - DNA, 54 bp, synthetic construct
  source 1..54: mol_type = other DNA
tggagttcagacgtgtgctcttccgatcttgaatggattccttggaaacaatga

SEQ ID NO: 133 - DNA, 60 bp, synthetic construct
  source 1..60: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnaatgtgtcaactcttgacagggc

SEQ ID NO: 134 - DNA, 55 bp, synthetic construct
  source 1..55: mol_type = other DNA
gactggagttcagacgtgtgctcttccgatctgagctgcaggtgtaatgaagacc

SEQ ID NO: 135 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnccgacagccagtggttaagt

SEQ ID NO: 136 - DNA, 49 bp, synthetic construct
  source 1..49: mol_type = other DNA
tggagttcagacgtgtgctcttccgatctgcttttcaccgactgcacag

SEQ ID NO: 137 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnagagactgattgcgtggagt

SEQ ID NO: 138 - DNA, 49 bp, synthetic construct
  source 1..49: mol_type = other DNA
tggagttcagacgtgtgctcttccgatctcactccagcctaggcaacaa

SEQ ID NO: 139 - DNA, 60 bp, synthetic construct
  source 1..60: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnagcatgcatttgtaggcttgatg

SEQ ID NO: 140 - DNA, 49 bp, synthetic construct
  source 1..49: mol_type = other DNA
tggagttcagacgtgtgctcttccgatctcccagccaaacttgtcaacc

SEQ ID NO: 141 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnttgcttattgctgaggggca

SEQ ID NO: 142 - DNA, 49 bp, synthetic construct
  source 1..49: mol_type = other DNA
tggagttcagacgtgtgctcttccgatctacctctctcctccagctgag

SEQ ID NO: 143 - DNA, 60 bp, synthetic construct
  source 1..60: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnggcttatgaaggcagagactgag

SEQ ID NO: 144 - DNA, 52 bp, synthetic construct
  source 1..52: mol_type = other DNA
tggagttcagacgtgtgctcttccgatctgttacctctcctttccaaggcac

SEQ ID NO: 145 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnngtctgaggtcacacagtggg

SEQ ID NO: 146 - DNA, 49 bp, synthetic construct
  source 1..49: mol_type = other DNA
tggagttcagacgtgtgctcttccgatctctgagagcagggaccacatc

SEQ ID NO: 147 - DNA, 60 bp, synthetic construct
  source 1..60: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnntacacccaattcttcactgatgc

SEQ ID NO: 148 - DNA, 57 bp, synthetic construct
  source 1..57: mol_type = other DNA
gactggagttcagacgtgtgctcttccgatctcaaacaaacgttatgacaaacctcc

SEQ ID NO: 149 - DNA, 60 bp, synthetic construct
  source 1..60: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnggtgattcaaagggtatcaggcc

SEQ ID NO: 150 - DNA, 58 bp, synthetic construct
  source 1..58: mol_type = other DNA
gactggagttcagacgtgtgctcttccgatctggcactcataaacagaaggttctacc

SEQ ID NO: 151 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnncctggcctcactggatactc

SEQ ID NO: 152 - DNA, 52 bp, synthetic construct
  source 1..52: mol_type = other DNA
tggagttcagacgtgtgctcttccgatctgaatgactgaatcggaacaaggc

SEQ ID NO: 153 - DNA, 56 bp, synthetic construct
  source 1..56: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnctagccttgcgttccgagg

SEQ ID NO: 154 - DNA, 48 bp, synthetic construct
  source 1..48: mol_type = other DNA
tggagttcagacgtgtgctcttccgatctcctgcagtccccaagatcg

SEQ ID NO: 155 - DNA, 58 bp, synthetic construct
  source 1..58: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnagggtgcttgagttgatcctg

SEQ ID NO: 156 - DNA, 48 bp, synthetic construct
  source 1..48: mol_type = other DNA
tggagttcagacgtgtgctcttccgatctatgctggcctcagctggtg

SEQ ID NO: 157 - DNA, 58 bp, synthetic construct
  source 1..58: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnncctcacagaaggatgtcggag

SEQ ID NO: 158 - DNA, 48 bp, synthetic construct
  source 1..48: mol_type = other DNA
tggagttcagacgtgtgctcttccgatcttgcctgtagtgctgacgtc

SEQ ID NO: 159 - DNA, 60 bp, synthetic construct
  source 1..60: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnntgctgcaagtaagcatgcatttg

SEQ ID NO: 160 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnncagctcagcctgagtgttga

SEQ ID NO: 161 - DNA, 47 bp, synthetic construct
  source 1..47: mol_type = other DNA
tggagttcagacgtgtgctcttccgatctctgcttcgtggcaatgcg

SEQ ID NO: 162 - DNA, 59 bp, synthetic construct
  source 1..59: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnncctgcagtctcctgcttctctg

SEQ ID NO: 163 - DNA, 55 bp, synthetic construct
  source 1..55: mol_type = other DNA
gactggagttcagacgtgtgctcttccgatctaacccagatgagaggatgaaggc

SEQ ID NO: 164 - DNA, 58 bp, synthetic construct
  source 1..58: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnggacatttccaccgcaaaatg

SEQ ID NO: 165 - DNA, 47 bp, synthetic construct
  source 1..47: mol_type = other DNA
tggagttcagacgtgtgctcttccgatgctacagaaaggtcagcagc

SEQ ID NO: 166 - DNA, 59 bp, synthetic construct
  source 1..59: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnngctgtggcatccagagacatgg

SEQ ID NO: 167 - DNA, 52 bp, synthetic construct
  source 1..52: mol_type = other DNA
tggagttcagacgtgtgctcttccgatctctctttgctccagatttcccttc

SEQ ID NO: 168 - DNA, 60 bp, synthetic construct
  source 1..60: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnngaatcctggacaaggtttgaagg

SEQ ID NO: 169 - DNA, 49 bp, synthetic construct
  source 1..49: mol_type = other DNA
tggagttcagacgtgtgctcttccgatcttcctgaggtctaggaacccg

SEQ ID NO: 170 - DNA, 60 bp, synthetic construct
  source 1..60: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnncatgaaactgtagccccagctac

SEQ ID NO: 171 - DNA, 55 bp, synthetic construct
  source 1..55: mol_type = other DNA
tggagttcagacgtgtgctcttccgatctacttggaaccaacccaaatattcctc

SEQ ID NO: 172 - DNA, 60 bp, synthetic construct
  source 1..60: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnncactggcctttattcagtccctc

SEQ ID NO: 173 - DNA, 51 bp, synthetic construct
  source 1..51: mol_type = other DNA
tggagttcagacgtgtgctcttccgatctagagcactgagcatagaccaag

SEQ ID NO: 174 - DNA, 59 bp, synthetic construct
  source 1..59: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnngctttaaacatttgtctgtgcg

SEQ ID NO: 175 - DNA, 51 bp, synthetic construct
  source 1..51: mol_type = other DNA
tggagttcagacgtgtgctcttccgatctgttttctgtccctccctcagta

SEQ ID NO: 176 - DNA, 58 bp, synthetic construct
  source 1..58: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnncagagagagcaggacgtcaca

SEQ ID NO: 177 - DNA, 51 bp, synthetic construct
  source 1..51: mol_type = other DNA
tggagttcagacgtgtgctcttccgatctagcactacctacgtcagcacct

SEQ ID NO: 178 - DNA, 56 bp, synthetic construct
  source 1..56: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnttctgctcggactcaggcc

SEQ ID NO: 179 - DNA, 53 bp, synthetic construct
  source 1..53: mol_type = other DNA
tggagttcagacgtgtgctcttccgatctaaccctatgtagcctcagtcttcc

SEQ ID NO: 180 - DNA, 59 bp, synthetic construct
  source 1..59: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnngacagagggagagaaacagagc

SEQ ID NO: 181 - DNA, 51 bp, synthetic construct
  source 1..51: mol_type = other DNA
tggagttcagacgtgtgctcttccgatctttctagatgccgacaaaaggat

SEQ ID NO: 182 - DNA, 59 bp, synthetic construct
  source 1..59: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnncactgctgaaccagtcaaactc

SEQ ID NO: 183 - DNA, 51 bp, synthetic construct
  source 1..51: mol_type = other DNA
tggagttcagacgtgtgctcttccgatctggcatggggaaatataaacttg

SEQ ID NO: 184 - DNA, 59 bp, synthetic construct
  source 1..59: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnncagagtctagagggcagtggtg

SEQ ID NO: 185 - DNA, 53 bp, synthetic construct
  source 1..53: mol_type = other DNA
tggagttcagacgtgtgctcttccgatctctcccacacacattgaatctcctg

SEQ ID NO: 186 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnctgactcagccctgcaaagg

SEQ ID NO: 187 - DNA, 48 bp, synthetic construct
  source 1..48: mol_type = other DNA
tggagttcagacgtgtgctcttccgatctcaagtcaggggagcgtgtc

SEQ ID NO: 188 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnacgtctcatatgccccttgg

SEQ ID NO: 189 - DNA, 51 bp, synthetic construct
  source 1..51: mol_type = other DNA
tggagttcagacgtgtgctcttccgatctacgtaggaattttggtgggaca

SEQ ID NO: 190 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnccctgttcctaaagcccacc

SEQ ID NO: 191 - DNA, 49 bp, synthetic construct
  source 1..49: mol_type = other DNA
tggagttcagacgtgtgctcttccgatctactggttctgtttgtggcca

SEQ ID NO: 192 - DNA, 55 bp, synthetic construct
  source 1..55: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnccagggaaacgcccatgc

SEQ ID NO: 193 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnaaaaggggcaagcttcagat

SEQ ID NO: 194 - DNA, 49 bp, synthetic construct
  source 1..49: mol_type = other DNA
tggagttcagacgtgtgctcttccgatctagtgaggagaaggcaggagg

SEQ ID NO: 195 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnntgttctgccctcacagaggt

SEQ ID NO: 196 - DNA, 47 bp, synthetic construct
  source 1..47: mol_type = other DNA
tggagttcagacgtgtgctcttccgatcccaaaggacatacggggag

SEQ ID NO: 197 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnngtgcgtgcttcttacatgcc

SEQ ID NO: 198 - DNA, 51 bp, synthetic construct
  source 1..51: mol_type = other DNA
tggagttcagacgtgtgctcttccgatccaagtatgccttaagcagaacaa

SEQ ID NO: 199 - DNA, 58 bp, synthetic construct
  source 1..58: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnctgtctcagttctcagtccgg

SEQ ID NO: 200 - DNA, 53 bp, synthetic construct
  source 1..53: mol_type = other DNA
gactggagttcagacgtgtgctcttccgatcttagctctgccttatggggagg

SEQ ID NO: 201 - DNA, 58 bp, synthetic construct
  source 1..58: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnngtgtggagagtgagtaagcca

SEQ ID NO: 202 - DNA, 50 bp, synthetic construct
  source 1..50: mol_type = other DNA
tggagttcagacgtgtgctcttccgatctacggtaggatgatttcaggca

SEQ ID NO: 203 - DNA, 58 bp, synthetic construct
  source 1..58: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnncacaaagcagtgtagctcagg

SEQ ID NO: 204 - DNA, 54 bp, synthetic construct
  source 1..54: mol_type = other DNA
tggagttcagacgtgtgctcttccgatcttttttggtactcgagtgttattcag

SEQ ID NO: 205 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnntcccctgttgacctggagaa

SEQ ID NO: 206 - DNA, 49 bp, synthetic construct
  source 1..49: mol_type = other DNA
tggagttcagacgtgtgctcttccgatctcactgtacttgccctgacca

SEQ ID NO: 207 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnttggtgttgacagggagcaa

SEQ ID NO: 208 - DNA, 49 bp, synthetic construct
  source 1..49: mol_type = other DNA
tggagttcagacgtgtgctcttccgatctctgagatgtgggcagaaggg

SEQ ID NO: 209 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnntgagagggaacagaagggct

SEQ ID NO: 210 - DNA, 49 bp, synthetic construct
  source 1..49: mol_type = other DNA
tggagttcagacgtgtgctcttccgatctgtccaaaggcccaagaacct

SEQ ID NO: 211 - DNA, 59 bp, synthetic construct
  source 1..59: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnngggagatttgcatctgtggagg

SEQ ID NO: 212 - DNA, 55 bp, synthetic construct
  source 1..55: mol_type = other DNA
tggagttcagacgtgtgctcttccgatctgcttttataccatcttggggttacag

SEQ ID NO: 213 - DNA, 60 bp, synthetic construct
  source 1..60: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnncaatgtgcttcaacccatcacgg

SEQ ID NO: 214 - DNA, 56 bp, synthetic construct
  source 1..56: mol_type = other DNA
tggagttcagacgtgtgctcttccgatctccatgaatttgtgatggatgcagtctg

SEQ ID NO: 215 - DNA, 57 bp, synthetic construct
  source 1..57: mol_type = other DNA
  misc_difference 34: note = g, a, t, or c
  misc_difference 35: note = g, a, t, or c
  misc_difference 36: note = g, a, t, or c
  misc_difference 37: note = g, a, t, or c
acactctttccctacacgacgctcttccgatctnnnnaggaggtgcaggagctagac

SEQ ID NO: 216 - DNA, 51 bp, synthetic construct
  source 1..51: mol_type = other DNA
tggagttcagacgtgtgctcttccgatcttcctcgtcctgctctcacttag

SEQ ID NO: 217 - DNA, 20 bp, synthetic construct
  source 1..20: mol_type = other DNA
gaacacaaagcatagactgc

SEQ ID NO: 218 - DNA, 20 bp, synthetic construct
  source 1..20: mol_type = other DNA
aagtgtgatcacttgggtgg

SEQ ID NO: 219 - DNA, 20 bp, synthetic construct
  source 1..20: mol_type = other DNA
caggacggtcacctttgggg

SEQ ID NO: 220 - DNA, 20 bp, synthetic construct
  source 1..20: mol_type = other DNA
aggatgcaagtttgtcttgg

SEQ ID NO: 221 - DNA, 20 bp, synthetic construct
  source 1..20: mol_type = other DNA
ggtacctatcgattgtcagg

SEQ ID NO: 222 - DNA, 20 bp, synthetic construct
  source 1..20: mol_type = other DNA
gagtatgaggcatagactgc

SEQ ID NO: 223 - DNA, 20 bp, synthetic construct
  source 1..20: mol_type = other DNA
ggattgacccaggccagggc

SEQ ID NO: 224 - DNA, 20 bp, synthetic construct
  source 1..20: mol_type = other DNA
gaagaccaaggatagactgc

SEQ ID NO: 225 - DNA, 20 bp, synthetic construct
  source 1..20: mol_type = other DNA
agccaggacggtcacctttg

SEQ ID NO: 226 - DNA, 20 bp, synthetic construct
  source 1..20: mol_type = other DNA
gacaagtgtgatcacttggg

SEQ ID NO: 227 - DNA, 20 bp, synthetic construct
  source 1..20: mol_type = other DNA
ccaaggattgacccaggcca

SEQ ID NO: 228 - DNA, 20 bp, synthetic construct
  source 1..20: mol_type = other DNA
ccaagtacttcaggtagctg

SEQ ID NO: 229 - DNA, 20 bp, synthetic construct
  source 1..20: mol_type = other DNA
gatgagataatgatgagtca

SEQ ID NO: 230 - DNA, 20 bp, synthetic construct
  source 1..20: mol_type = other DNA
aagcaattgttatgattaaa

SEQ ID NO: 231 - DNA, 20 bp, synthetic construct
  source 1..20: mol_type = other DNA
aatacattccaaaagaaatg

SEQ ID NO: 232 - DNA, 20 bp, synthetic construct
  source 1..20: mol_type = other DNA
gaatacattccaaaagaaat

SEQ ID NO: 233 - DNA, 20 bp, synthetic construct
  source 1..20: mol_type = other DNA
tgaatacattccaaaagaaa

SEQ ID NO: 234 - DNA, 20 bp, synthetic construct
  source 1..20: mol_type = other DNA
atatttgcattgagatagtg

SEQ ID NO: 235 - DNA, 20 bp, synthetic construct
  source 1..20: mol_type = other DNA
ccaccgctgcgccaaggtgc

SEQ ID NO: 236 - DNA, 20 bp, synthetic construct
  source 1..20: mol_type = other DNA
taaggcccaagggggcaagc

SEQ ID NO: 237 - DNA, 20 bp, synthetic construct
  source 1..20: mol_type = other DNA
gcaggtgaccgtggcctgcg

SEQ ID NO: 238 - DNA, 20 bp, synthetic construct
  source 1..20: mol_type = other DNA
ggcccagactgagcacgtga

SEQ ID NO: 239 - DNA, 20 bp, synthetic construct
  source 1..20: mol_type = other DNA
gagtccgagcagaagaagaa

SEQ ID NO: 240 - DNA, 22 bp, synthetic construct
  source 1..22: mol_type = other DNA
gtggtagacagcatgtgtccta

SEQ ID NO: 241 - DNA, 23 bp, synthetic construct
  source 1..23: mol_type = other DNA
gatttacagcctggcctttgggg

SEQ ID NO: 242 - DNA, 22 bp, synthetic construct
  source 1..22: mol_type = other DNA
gtgtcaggtaatgtgctaaaca

SEQ ID NO: 243 - DNA, 22 bp, synthetic construct
  source 1..22: mol_type = other DNA
ggtggaggagggtgcatggggt

SEQ ID NO: 244 - DNA, 22 bp, synthetic construct
  source 1..22: mol_type = other DNA
ggcaagaggattgattgagcca

SEQ ID NO: 245 - DNA, 22 bp, synthetic construct
  source 1..22: mol_type = other DNA
actagtgtgcgaagtatcataa

SEQ ID NO: 246 - DNA, 20 bp, synthetic construct
  source 1..20: mol_type = other DNA
ggttaacaaggccaaactcc

SEQ ID NO: 247 - DNA, 20 bp, synthetic construct
  source 1..20: mol_type = other DNA
gggtccagttccgggattag

SEQ ID NO: 248 - DNA, 20 bp, synthetic construct
  source 1..20: mol_type = other DNA
caaggatagactgctgggct

SEQ ID NO: 249 - RNA, 20 nt, synthetic construct
  source 1..20: mol_type = other RNA
gaggacaaagtacaaacggc

SEQ ID NO: 250 - DNA, 20 bp, synthetic construct
  source 1..20: mol_type = other DNA
gatgacccgtattatctggc

SEQ ID NO: 251 - DNA, 20 bp, synthetic construct
  source 1..20: mol_type = other DNA
ggagacatcaaaccatgact

SEQ ID NO: 252 - DNA, 20 bp, synthetic construct
  source 1..20: mol_type = other DNA
caagtgatcacacttgtcac

SEQ ID NO: 253 - DNA, 20 bp, synthetic construct
  source 1..20: mol_type = other DNA
atagactgcaggagacatca

SEQ ID NO: 254 - DNA, 20 bp, synthetic construct
  source 1..20: mol_type = other DNA
atgacttgcagatgaagaag

SEQ ID NO: 255 - DNA, 20 bp, synthetic construct
  source 1..20: mol_type = other DNA
gattcaaagccatttttcca

SEQ ID NO: 256 - DNA, 22 bp, synthetic construct
  source 1..22: mol_type = other DNA
tacagagggacagaggcctgac

SEQ ID NO: 257 - DNA, 22 bp, synthetic construct
  source 1..22: mol_type = other DNA
atgagaagtatgacaacagcct

SEQ ID NO: 258 - DNA, 22 bp, synthetic construct
  source 1..22: mol_type = other DNA
ggcagtcatcttagtcattacc

SEQ ID NO: 259 - DNA, 22 bp, synthetic construct
  source 1..22: mol_type = other DNA
ggactagagtaggattgtaccc

SEQ ID NO: 260 - DNA, 22 bp, synthetic construct
  source 1..22: mol_type = other DNA
ggctgagctaactgtgacagca

SEQ ID NO: 261 - DNA, 23 bp, synthetic construct
  source 1..23: mol_type = other DNA
tgctgcaagtaagcatgcatttg

SEQ ID NO: 262 - DNA, 23 bp, synthetic construct
  source 1..23: mol_type = other DNA
ctagacaggggctagtatgtgca

SEQ ID NO: 263 - DNA, 23 bp, synthetic construct
  source 1..23: mol_type = other DNA
cagctattcaggctggcccgccc

SEQ ID NO: 264 - DNA, 23 bp, synthetic construct
  source 1..23: mol_type = other DNA
gaagcacatcaaggacattctaa

SEQ ID NO: 265 - DNA, 23 bp, synthetic construct
  source 1..23: mol_type = other DNA
ggataagcacagttttaaatagt

SEQ ID NO: 266 - DNA, 23 bp, synthetic construct
  source 1..23: mol_type = other DNA
gtttaaacacaccgggttaataa

SEQ ID NO: 267 - DNA, 20 bp, synthetic construct
  source 1..20: mol_type = other DNA
tgtcgaagttcgccctggag

SEQ ID NO: 268 - DNA, 20 bp, synthetic construct
  source 1..20: mol_type = other DNA
cgaagttcgccctggagagg

SEQ ID NO: 269 - DNA, 20 bp, synthetic construct
  source 1..20: mol_type = other DNA
gctctgatctgaataccacg

SEQ ID NO: 270 - DNA, 20 bp, synthetic construct
  source 1..20: mol_type = other DNA
gaacacaatgcatagattgc

SEQ ID NO: 271 - DNA, 20 bp, synthetic construct
  source 1..20: mol_type = other DNA
aaacataaagcatagactgc

SEQ ID NO: 272 - DNA, 20 bp, synthetic construct
  source 1..20: mol_type = other DNA
cacccagactgagcacgtgc

SEQ ID NO: 273 - DNA, 20 bp, synthetic construct
  source 1..20: mol_type = other DNA
gacacagaccgggcacgtga

SEQ ID NO: 274 - DNA, 20 bp, synthetic construct
  source 1..20: mol_type = other DNA
agctcagactgagcaagtga

SEQ ID NO: 275 - DNA, 20 bp, synthetic construct
  source 1..20: mol_type = other DNA
agaccagactgagcaagaga

SEQ ID NO: 276 - DNA, 20 bp, synthetic construct
  source 1..20: mol_type = other DNA
gagttagagcagaagaagaa

SEQ ID NO: 277 - DNA, 20 bp, synthetic construct
  source 1..20: mol_type = other DNA
gagtctaagcagaagaagaa

SEQ ID NO: 278 - DNA, 20 bp, synthetic construct
  source 1..20: mol_type = other DNA
gaggccgagcagaagaaaga

SEQ ID NO: 279 - RNA, 19 nt, synthetic construct
  source 1..19: mol_type = other RNA
gtactcggctacgaaccag

SEQ ID NO: 280 - RNA, 20 nt, synthetic construct
  source 1..20: mol_type = other RNA
gtactcggctacgaaccgag

SEQ ID NO: 281 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 282 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 283 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 284 - protein, 1368 aa, synthetic construct
MDKKYSIGLTIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGD

SEQ ID NO: 285 - protein, 1368 aa, synthetic construct
MDKKYSIGLTIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIGFLEAKGYKEVKKDLIIKLPKYSLFELENGRKRMLASASVLHKGNELALPSKYVNFLYLASHYEKLKGSSEDNKQKQLFVEQHKHYLDEIIEQISEFSKRVILADANLDKVLSAYNKHRDKPIREQAENIIHLFTLTNLGASAAFKYFDTTIGRKLYTSTKEVLDATLIHQSITGLYETRIDLSQLGGD

SEQ ID NO: 286 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 287 - protein, 1054 aa, synthetic construct
MGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 288 - protein, 1054 aa, synthetic construct
MGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 289 - protein, 1227 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 290 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYRNRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNREKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIARGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 291 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARGAKTGAAGSLMDVLHHPGMNHRVEITEGILADECAALLSDFFRMRRQEIKAQKKAQSSTD

SEQ ID NO: 292 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARGAKTGAAGSLMDVLHHPGMNHRVEITEGILADECAALLSDFFRMRRQEIKAQKNAQSSTD

SEQ ID NO: 293 - protein, 166 aa, synthetic construct
SEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGARGARTGAAGSLMDVLRHPGIKHRVEITEGILADECAALLSDFFRMRRQEIKAQKNAQSSTD

SEQ ID NO: 294 - protein, 166 aa, synthetic construct
SEVEFSHEYWMRHALTLAKRAWDEREVPVGAVLVHNNRVIGEGWNRAIGHHDPTAHAEIMALRQGGLVMQNYRLIDATLYVTLEPCVMCAGAMIHSRIGRVVFGARGARTGAAGSLMDVLRHPGIKHRVEITEGILADECAALLSDFFRMRRQEIKAQKKAQSSTD

SEQ ID NO: 295 - protein, 166 aa, synthetic construct
SEVEFSHEYWMRHALTLAKRAWDERDVPVGAVLVHNNRVIGEGWNRAIGHHDPTAHAEIMALRQGGLVMQNYRLFDATLYVTLEPCVMCAGAMIHSRIGRVVFGARGARTGAAGSLMDVLRHPGIKHRVEITEGILADECAALLSDFFRMRRQEIKAQKNAQSSTD

SEQ ID NO: 296 - protein, 166 aa, synthetic construct
SEVEFSHEYWMRHALTLAKRAWDERDVPVGAVLVHNNRVIGEGWNKAIGHHDPTAHAEIMALRQGGLVMQNYRLFDATLYVTLEPCVMCAGAMIHSRIGRVVFGARGARTGAAGSLMDVLRHPGIKHRVEITEGILADECAALLSDFFRMRRQEIKAQKNAQSSTD

SEQ ID NO: 297 - protein, 166 aa, synthetic construct
SEVEFSHEYWMRHALTLAKRAWDERDVPVGAVLVHNNRVIGEGWNRAIGHHDPTAHAEIMALRQGGLVMQNYRLIDATLYVTLEPCVMCAGAMIHSRIGRVVFGARGARTGAVGSLMDVLRHPGIKHRVEITEGILADECAALLSDFFRMRRQEIKAQKKAQSSTD

SEQ ID NO: 298 - protein, 166 aa, synthetic construct
SEVEFSHEYWMRHALTLAKRAWDERDVPVGAVLVHNNRVIGEGWNKAIGHHDPTAHAEIMALRQGGLVMQNYRLFDATLYVTLEPCVMCAGAMIHSRIGRVVFGARGARTGAVGSLMDVLRHPGIKHRVEITEGILADECAALLSDFFRMRRQEIKAQKKAQSSTD

SEQ ID NO: 299 - protein, 166 aa, synthetic construct
SEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLSRFFRMPRRVFKAQKKAQSSTD

SEQ ID NO: 300 - protein, 166 aa, synthetic construct
SEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLRYPGIKHRVEITEGILADECAALLSRFFRMPRRVFKAQKKAQSSTD

SEQ ID NO: 301 - protein, 166 aa, synthetic construct
SEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLCRFFRMPRRVFKAQKKAQSSTD

SEQ ID NO: 302 - protein, 166 aa, synthetic construct
SEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLSRFFRMPRRVFNAQKKAQSSTD

SEQ ID NO: 303 - protein, 166 aa, synthetic construct
SEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLSRFFRMPRRVFKAQKNAQSSTD

SEQ ID NO: 304 - protein, 166 aa, synthetic construct
SEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLSRFFRMPRRVFKAQKKAQSSID

SEQ ID NO: 305 - protein, 166 aa, synthetic construct
SEVEFSHEYWMRHALTLAKRAWDEREVPVGAVLVHNNRVIGEGWNRAIGHHDPTAHAEIMALRQGGLVMQNYRLFDATLYVTLEPCVMCAGAMIHSRIGRVVFGVRGARHGAAGSLMNVLHYPGIKHRVEITEGILADECAALLSRFFRMPRRVFKAQKKAQSSTN

SEQ ID NO: 306 - protein, 165 aa, synthetic construct
SEVEFSHEYWMRHALTLAKRARDEREVPVGAVLVLNNRVIGEGWNKAIGLHDPTAHAEIMALRQGGLVMQNYRLFDATLYSTLEPCVMCAGAIHSRIGRVVFGVRGARHGAAGSLMNVLHYPGIKHRVEITEGILADECAALLCRFFRMPRRVFKAQKKAQSSTD

SEQ ID NO: 307 - protein, 166 aa, synthetic construct
SEVEFSHEYWMRHALTLAKRARDEREVPVGAVLVLNNRVIGEGWNKAIGLHDPTAHAEIMALRQGGLVMQNYRLFDATLYSTLEPCVMCAGAMIHSRIGRVVFGVRGARHGAAGSLMNVLNYPGIKHRVEITEGILADECAALLCRFFRMPRRVFKAQKKAQSSTD

SEQ ID NO: 308 - protein, 166 aa, synthetic construct
SEVEFSHEYWMRHALTLAKRARDEREVPVGAVLVLNNRVIGEGWNKAIGLHDPTAHAEIMALRQGGLVMQNYRLYDATLYSTLEPCVMCAGAMIHSRIGRVVFGVRGARHGAVGSLMNVLHYPGIKHRVEITEGILADECAALLCRFFRMPRRVFKAQKKAQSSTD

SEQ ID NO: 309 - protein, 166 aa, synthetic construct
SEVEFSHEYWMRHALTLAKRARDEREVPVGAVLVLNNRVIGEGWNKAIGLHDPTAHAEIMALRQGGLVMQNYRLFDATLYSTLEPCVMCAGAMIHSRIGRVVFGVRGSRHGAVGSLMNVLHYPGIKHRVEITEGILADECAALLSRFFRMPRRVFKAQKKAQSSTD

SEQ ID NO: 310 - protein, 166 aa, synthetic construct
SEVEFSHEYWMRHALTLAKRARDEREVPVGAVLVHNNRVIGEGWNKAIGLHDPTAHAEIMALRQGGLVMQNYRLYDATLYSTLEPCVMCAGAMIHSRIGRVVFGVRGSRHGAVGSLMNVLHYPGIKHRVEITEGILADECAALLSRFFRMPRRVFKAQKKAQSSTD

SEQ ID NO: 311 - protein, 166 aa, synthetic construct
SEVEFSHEYWMRHALTLAKRARDEREVPVGAVLVLNNRVIGEGWNKAIGLHDPTAHAEIMALRQGGLVMQNYRLYDATLYSTLEPCVMCAGAMIHSRIGRVVFGVRGARHGAVGSLMNVLHYPGIKHRVEITEGILADECAALLCRFFRMPRRVFNAQKNAQSSIN

SEQ ID NO: 312 - protein, 166 aa, synthetic construct
SEVEFSHEYWMRHALTLAKRARDEREVPVGAVLVLNNRVIGEGWNKAIGLHDPTAHAEIMALRQGGLVMQNYRLYDATLYSTLEPCVMCAGAMIHSRIGRVVFGVRGARHGAVGSLMNVLHYPGIKHRVEITEGILADECAALLCRFFRMPRRVFKAQKKAQSSIN

SEQ ID NO: 313 - protein, 19 aa, synthetic construct
MKRTADGSEFESPKKKRKV

SEQ ID NO: 314 - protein, 32 aa, synthetic construct
SGGSSGGSSGSETPGTSESATPESSGGSSGGS

SEQ ID NO: 315 - protein, 4 aa, synthetic construct
SGGS

SEQ ID NO: 316 - protein, 17 aa, synthetic construct
KRTADGSEFEPKKKRKV

SEQ ID NO: 317 - protein, 1605 aa, synthetic construct
MKRTADGSEFESPKKKRKVSEVEFSHEYWMRHALTLAKRARDEREVPVGAVLVLNNRVIGEGWNKAIGLHDPTAHAEIMALRQGGLVMQNYRLYDATLYSTLEPCVMCAGAMIHSRIGRVVFGVRGARHGAVGSLMNVLHYPGIKHRVEITEGILADECAALLCRFFRMPRRVFKAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

(19) United States
(12) Patent Application Publication
(10) Pub. No.: US 2024/0352439 A1
(43) Pub. Date: Oct. 24, 2024

(54) POLYPEPTIDES AND METHODS FOR MODIFYING NUCLEIC ACIDS

(71) Applicant: THE UNIVERSITY OF CHICAGO, Chicago, IL (US)

(72) Inventors: Weixin TANG, Chicago, IL (US); Yulan XIAO, Chicago, IL (US)

(73) Assignee: THE UNIVERSITY OF CHICAGO, Chicago, IL (US)

(21) Appl. No.: 18/688,268

(22) PCT Filed: Sep. 2, 2022
(86) PCT No.: PCT/US2022/075891

§ 371 Date: Feb. 29, 2024

Related U.S. Application Data
(60) Provisional application No. 63/240,525, filed on Sep. 3, 2021.

(51) Int. Cl. C12N 9/78; C12N 9/22; C12N 15/10

(52) U.S. Cl. CPC C12N 9/78; C12N 9/22; C12N 15/1058; C12Y 305/04004

(57) ABSTRACT

The inventors have made TadA variants with improved activities, such as improved based editing in certain genomic contexts and altered editing window. Aspects of the disclosure relate to a polypeptide comprising SEQ ID NO: 1, wherein the polypeptide comprises one or more amino acid substitutions relative to SEQ ID NO: 1, wherein the one or more amino acid substitutions comprise a substitution at amino acid (23, 27, 36, 47, 48, 51, 76, 82, 106, 108, 109,110, 111, 114, 119, 122, 123, 126, 127, 146, 147, 152, 154, 155, 156, 157, 161, 166, 167), and combinations thereof.

### CROSS-REFERENCE TO RELATED APPLICATIONS

This application claims the benefit of priority of U.S. Provisional Patent Application No. 63/240,525 filed Sep. 3, 2021, which is hereby incorporated by reference in its entirety.

### BACKGROUND OF THE INVENTION

### II. Field of the Invention

This invention relates to the field of molecular biology

### III. Background

Approximately 60% of known disease-associated genetic variations in the human genome are point mutations, close to half of which are G:C to A:T transitions (1, 2). Adenine base editors (ABEs), wherein a deoxyadenosine deaminase is covalently linked to a catalytically impaired CRISPR protein via a flexible linker, can correct G:C to A:T mutations site-specifically in the genome without introducing excessive double-stranded DNA (dsDNA) breaks (3, 4). The deoxyadenosine deaminases in ABEs are variants of the Escherichia coli tRNA-specific adenosine deaminase (TadA) (5) evolved to function on single-stranded DNA (ssDNA). While ABE activity was initially demonstrated with Streptococcus pyogenes Cas9 (SpCas9), more CRISPR proteins have since been demonstrated compatible with TadA variants for adenine base editing (6-8). Correction of disease relevant mutations has been achieved with ABEs in a variety of cell models and organisms (9-17), including non-human primates (18-20).

Seven rounds of directed evolution in E. coli yielded TadA7.10 (3), the TadA variant that comprises the state-of-the-art ABE-ABE7.10—wherein a SpCas9 nickase (nCas9) is employed for target DNA engagement. ABE7.10 edits A into G in a window spanning protospacer positions 4-7 through an inosine (I) intermediate. TadA7.10 is most efficient in deaminating A in a “YA” motif (Y: pyrimidine; T and C) (3), a context preference inherited from WT TadA that deaminates adenosine in the anti-codon loop (U)ACG of Arg tRNA (5). This context bias is most evident when the target A is outside the strong editing window (21). TadA7.10, as is evolved in a SpCas9-guided manner, is less compatible with other CRISPR systems. More active TadA variants, TadA8 (22) and TadA8e (7), were obtained by pushing TadA7.10 through additional rounds of directed evolution with increased selection stringencies. ABE8e is 590-fold faster than ABE7.10 under single turnover conditions (7). With substantially improved deamination activity, ABE8 and ABE8e demonstrated universally higher activity and a broadened editing window (4-8) in human cells (7, 22). These high-activity ABEs can be particularly useful for editing disease-causing mutations in primary cells and in vivo where superior activity is required to compensate deficiency in delivery.

TadA8 and TadA8e, both of which are derivatives of TadA7.10, have inherited the weak “YA” context preference (7, 22, 23). Adenine following a purine (RA, R=A or G) remains a challenging substrate, especially when the target A is outside the most optimal editing window (5-7). Thus, there is a need in the art for the development of base editors with improved activities.

### SUMMARY OF THE INVENTION

The inventors have made TadA variants with improved activities, such as improved based editing in certain genomic contexts and altered editing window. Aspects of the disclosure relate to a polypeptide comprising SEQ ID NO:1, wherein the polypeptide comprises one or more amino acid substitutions relative to SEQ ID NO:1, wherein the one or more amino acid substitutions comprise a substitution at amino acid 23, 27, 36, 47, 48, 51, 76, 82, 106, 108, 109, 110, 111, 114, 119, 122, 123, 126, 127, 146, 147, 152, 154, 155, 156, 157, 161, 166, 167, and combinations thereof. Also described is a nucleic acid encoding a polypeptide of the disclosure, an expression vector comprising the nucleic acid, and host cells comprising the polypeptide, expression vector, and/or nucleic acid of the disclosure. Further aspects relate to a method for making a polypeptide comprising transferring the expression vector of the disclosure into a cell under conditions sufficient for expression of the polypeptide encoded on the expression vector. Further aspects relate to a method for modifying adenine bases and/or for editing adenine bases in a nucleic acid molecule comprising contacting the nucleic acid with a polypeptide of the disclosure.

Yet further aspects relate to a method for directed evolution of an editor, the method comprising: (i) generating a library of variant genes of the editor by mutagenesis; (ii) selecting or screening for one or more variants with increased fitness, wherein each variant comprises one or more substitutions in the amino acid sequence of the editor; (iii) generating a library of variant genes by mutagenesis, wherein the template variant genes comprises the one or more variants with increased fitness; (iv) selecting or screening for one or more variants with increased fitness, wherein each variant comprises one or more substitutions in the amino acid sequence of the editor; (v) repeating steps (iii) and (iv) iteratively between 0-10 additional times; (vi) generating a library of variant genes; wherein the library comprises variant genes that combines the one or more substitutions of the selected variants of (iv) or (v); (vii) selecting or screening for one or more variants with increased fitness; wherein each variant comprises one or more substitutions in the amino acid sequence of the editor; and (viii) repeating steps (iii) and (iv) or steps (vi) and (vii) iteratively between 0-10 additional times. In some aspects, the method comprises (i) generating a library of variant genes; wherein the library comprises a combinatorial library; (ii) selecting or screening for one or more variants with increased fitness; wherein each variant comprises one or more substitutions in the amino acid sequence of the editor; and (iii) repeating steps (i) and (ii) iteratively between 0-10 additional times.

In some aspects, the one or more amino acid substitutions comprise one or more of W23R, E27D, H36L, R47K, P48A, R51H, R51L, I76F, I76Y, V82S, A106V, D108G, A109S, K110R, T111H, A114V, D119N, H122R, H122N, H123Y, M126I, N127K, S146C, D147R, R152P, Q154R, E155V, I156F, K157N, K161N, T166I, and/or D167N,

In some aspects, the polypeptide comprises a R47K substitution. In some aspects, the polypeptide is not substituted at amino acid 84, 109, 122, 149, and/or 157. In some aspects, the polypeptide does not have a substation at amino acid 84 and/or amino acid 149 of the TadA protein (SEQ ID NO:1). In some aspects, the polypeptide comprises a D108G substitution. In some aspects, the polypeptide is not substituted at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, and/or 167 of SEQ ID NO:1.

In some aspects, the polypeptide comprises a K110R substitution. In some aspects, the polypeptide comprises a T111H substitution. In some aspects, the polypeptide comprises a T111R substitution. In some aspects, the polypeptide comprises a A114V substitution. In some aspects, the polypeptide comprises a M126I substitution. In some aspects, the polypeptide comprises a N127K substitution. In some aspects, the polypeptide comprises a W23R substitution. In some aspects, the polypeptide comprises a E27D substitution. In some aspects, the polypeptide comprises a H36L substitution. In some aspects, the polypeptide comprises a P48A substitution. In some aspects, the polypeptide comprises a R51H substitution. In some aspects, the polypeptide comprises a R51L substitution. In some aspects, the polypeptide comprises a I76F substitution. In some aspects, the polypeptide comprises a I76Y substitution. In some aspects, the polypeptide comprises a V82S substitution. In some aspects, the the polypeptide comprises a A106V substitution. In some aspects, the polypeptide comprises a A109S substitution. In some aspects, the polypeptide comprises a D119N substitution. In some aspects, the polypeptide comprises a H122R substitution. In some aspects, the polypeptide comprises a H122N substitution. In some aspects, the polypeptide comprises a H123Y substitution. In some aspects, the polypeptide comprises a M126I substitution. In some aspects, the polypeptide comprises a S146C substitution. In some aspects, the polypeptide comprises a D147R substitution. In some aspects, the polypeptide comprises a R152P substitution. In some aspects, the polypeptide comprises a Q154R substitution. In some aspects, the polypeptide comprises a E155V substitution. In some aspects, the polypeptide comprises a I156F substitution. In some aspects, the polypeptide comprises a K157N substitution. In some aspects, the polypeptide comprises a K161N substitution. In some aspects, the polypeptide comprises a T166I substitution. In some aspects, the polypeptide comprises a D167N substitution.

In some aspects, the one or more substitutions comprise or consist of D108G and K161N substitutions. In some aspects, the one or more substitutions comprise or consist of P48A, D108G, and K161N substitutions. In some aspects, the one or more substitutions comprise or consist of P48A, I76F, D108G, and K161N substitutions. In some aspects, the one or more substitutions comprise or consist of P48A, R51H, I76F, D108G, K110R, H122R, M126I, N127K, and K161N substitutions. In some aspects, the one or more substitutions comprise or consist of P48A, R51H, D108G, K110R, H122R, M126I, and N127K, substitutions. In some aspects, the one or more substitutions comprise or consist of E27D, P48A, R51H, I76F, D108G, K110R, H122R, M126I, N127K, and K161N substitutions. In some aspects, the one or more substitutions comprise or consist of E27D, R47K, P48A, R51H, I76F, D108G, K110R, H122R, M126I, N127K, and K161N substitutions. In some aspects, the one or more substitutions comprise or consist of E27D, P48A, R51H, D108G, K110R, A114V, H122R, M126I, and N127K substitutions. In some aspects, the one or more substitutions comprise or consist of E27D, R47K, P48A, R51H, I76F, D108G, K110R, A114V, H122R, M126I, and N127K substitutions. In some aspects, the one or more substitutions comprise or consist of P48A, R51H, I76F, A106V, D108G, K110R, T111H, D119N, H123Y, M126I, N127K, D147R, R152P, Q154R, E155V, and I156F substitutions. In some aspects, the one or more substitutions comprise or consist of P48A, R51H, 176F, A106V, D108G, K110R, T111H, D119N, H122R, H123Y, M126I, N127K, D147R, R152P, Q154R, E155V, and I156F substitutions. In some aspects, the one or more substitutions comprise or consist of P48A, R51H, I76F, A106V, D108G, K110R, T111H, D119N, H123Y, M126I, N127K, S146C, D147R, R152P, Q154R, E155V, and I156F substitutions. In some aspects, the one or more substitutions comprise or consist of P48A, R51H, I76F, A106V, D108G, K110R, T111H, D119N, H123Y, M126I, N127K, D147R, R152P, Q154R, E155V, I156F, and K157N substitutions, the one or more substitutions comprise or consist of P48A, R51H, 176F, A106V, D108G, K110R, T111H, D119N, H123Y, M126I, N127K, D147R, R152P, Q154R, E155V, I156F, and K161N substitutions, the one or more substitutions comprise or consist of P48A, R51H, I76F, A106V, D108G, K110R, T111H, D119N, H123Y, M126I, N127K, D147R, R152P, Q154R, E155V, I156F, and T166I substitutions. In some aspects, the one or more substitutions comprise or consist of P48A, R51H, I76F, A106V, D108G, K110R, T111H, D119N, H123Y, M126I, N127K, D147R, R152P, Q154R, E155V, I156F, and D167N substitutions. In some aspects, the one or more substitutions comprise or consist of W23R, H36L, R47K, P48A, R51L, I76F, V82S, A106V, D108G, K110R, T111H, D119N, H123Y, M126I, N127K, S146C, D147R, R152P, Q154R, E155V, and I156F substitutions. In some aspects, the one or more substitutions comprise or consist of W23R, H36L, R47K, P48A, R51L, I76F, V82S, A106V, D108G, K110R, T111H, D119N, H122N, H123Y, M126I, N127K, S146C, D147R, R152P, Q154R, E155V, and I156F substitutions. In some aspects, the one or more substitutions comprise or consist of W23R, H36L, R47K, P48A, R51L, I76Y, V82S, A106V, D108G, K110R, T111H, A114V, D119N, H123Y, M126I, N127K, S146C, D147R, R152P, Q154R, E155V, and I156F substitutions. In some aspects, the one or more substitutions comprise or consist of W23R, H36L, R47K, P48A, R51L, I76F, V82S, A106V, D108G, A109S, K110R, T111H, A114V, D119N, H123Y, M126I, N127K, D147R, R152P, Q154R, E155V, and I156F substitutions. In some aspects, the one or more substitutions comprise or consist of W23R, R47K, P48A, R51L, I76Y, V82S, A106V, D108G, A109S, K110R, T111H, A114V, D119N, H123Y, M126I, N127K, D147R, R152P, Q154R, E155V, and I156F substitutions. In some aspects, the one or more substitutions comprise or consist of W23R, H36L, R47K, P48A, R51L, I76Y, V82S, A106V, D108G, K110R, T111H, A114V, D119N, H123Y, M126I, N127K, S146C, D147R, R152P, Q154R, E155V, I156F, K157N, K161N, T166I, and D167N substitutions. In some aspects, the one or more substitutions comprise or consist of W23R, H36L, R47K, P48A, R51L, I76Y, V82S, A106V, D108G, K110R, T111H, A114V, D119N, H123Y, M126I, N127K, S146C, D147R, R152P, Q154R, E155V, I156F, T166I, and D167N substitutions. In some aspects, the one or more substitutions comprise or consist of P48A, D108G, M126I, and K161N substitutions. In some aspects, the one or more substitutions comprise or consist of P48A, D108G, N127K, and K161N substitutions. In some aspects, the one or more substitutions comprise or consist of P48A, I76F, D108G, K110R, N127K, and K161N substitutions. In some aspects, the one or more substitutions comprise or consist of P48A, R51H, I76F, D108G, K110R, M126I, N127K, and K161N substitutions. In some aspects, the one or more substitutions comprise or consist of D108G, K110R, N127K, and K161N substitutions. In some aspects, the one or more substitutions comprise or consist of P48A, R51H, I76F, A106V, D108G, K110R, T111H, D119N, H123Y, M126I, N127K, D147R, R152P, Q154R, E155V, and I156F substitutions. In some aspects, the one or more substitutions comprise or consist of P48A, R51H, I76F, A106V, D108G, K110R, T111H, D119N, H123Y, M126I, N127K, S146C, D147R, R152P, Q154R, E155V, and I156F substitutions. In some aspects, the one or more substitutions comprise or consist of P48A, R51H, I76F, A106V, D108G, K110R, T111H, D119N, H123Y, M126I, N127K, D147R, R152P, Q154R, E155V, I156F, and K157N substitutions. In some aspects, the one or more substitutions comprise or consist of P48A, R51H, I76F, A106V, D108G, K110R, T111H, D119N, H123Y, M126I, N127K, D147R, R152P, Q154R, E155V, I156F, and K161N substitutions. In some aspects, the one or more substitutions comprise or consist of P48A, R51H, I76F, A106V, D108G, K110R, T111H, D119N, H123Y, M126I, N127K, D147R, R152P, Q154R, E155V, I156F, and T166I substitutions.

In some aspects, the polypeptide comprises or consists of a polypeptide having the amino acid sequence of one of SEQ ID NOS:2-30 or 291-312. The polypeptide may comprise at least 70% sequence identity to SEQ ID NO:1. In some aspects, the polypeptide comprises or comprises at least 80% sequence identity to one of SEQ ID NOS:2-30 or 291-312. In some aspects, the comprises or comprises at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOS:2-30 or 291-312. In some aspects, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, and/or 167 is substituted. In some aspects, the substitution is with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, the polypeptide comprises at least 2 amino acid substitutions relative to SEQ ID NO:1. In some aspects, the polypeptide comprises, comprises at least, or comprises at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 substitutions, or any derivable range therein, relative to SEQ ID NO:1. In some aspects, the polypeptide comprises, comprises at least, or comprises at most 2, 3, 4, 5, 6, 7, 8, 9, or 10 substitutions, or any derivable range therein, relative to one of SEQ ID NOS:2-30 or 291-312. In some aspects, the substitutions are at amino acid positions 23, 27, 36, 47, 48, 51, 76, 82, 106, 108, 109, 110, 111, 114, 119, 122, 123, 126, 127, 146, 147, 152, 154, 155, 156, 157, 161, 166, and/or 167. The substitutions may be selected from W23R, E27D, H36L, R47K, P48A, R51H, R51L, I76F, I76Y, V82S, A106V, D108G, A109S, K110R, T111H, A114V, D119N, H122R, H122N, H123Y, M126I, N127K, S146C, D147R, R152P, Q154R, E155V, I156F, K157N, K161N, T166I, and D167N.

In aspects of the disclosure, the polypeptide modifies adenosine bases in a nucleic acid molecule. The nucleic acid molecule may be a RNA or a DNA molecule. In some aspects, the nucleic acid molecule is RNA. In some aspects, the nucleic acid molecule is DNA. In some aspects, the nucleic acid molecule is single-stranded. In some aspects, the nucleic acid molecule is double-stranded. In some aspects, the polypeptide is covalently linked to an effector protein. In some aspects, the effector protein comprises a Cas protein, or a variant thereof. In some aspects, the effector comprises a catalytically impaired Cas protein. In some aspects, the Cas protein comprises a Cas9 protein. The effector or Cas protein may be further defined as a Sp dCas9 (D10A, H840A), Sp nCas9 (D10A), Hf nCas9 (D10A), Sp VQR nCas9 (D10A), Sp VRER nCas9 (D10A), Sa nCas9 (D10A), Sa KKH nCas9 (D10A), dCas12a, SpCas9(D10A)-NG, xCas9 (D10A), Sp dCas9, Sp dCas9, Sp n xCas9 (D10A), Sa nCas9 (D10A), Sp Cas9-VRQR, SpCas9-NG, SpCas9-NRCH, SpCas9Nrth, LbCpf, enAsCpf, or SaKH nCas9 (D10A). These protein variants are known in the art and described in, for example, Rees H A, Liu D R. Base editing: precision chemistry on the genome and transcriptome of living cells. Nat Rev Genet. 2018 December; 19(12):770-788. doi: 10.1038/s41576-018-0059-1. Erratum in: Nat Rev Genet. 2018 Oct. 19; PMID: 30323312; PMCID: PMC6535181, which is herein incorporated by reference. In some aspects, the effector protein comprises an amino acid sequence of one of SEQ ID NOS:281-290 or an amino acid sequence with at least 80% sequence identity to one of SEQ ID NOS:281-290. In some aspects, the effector protein comprises an amino acid sequence that has or has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOS:281-290. The effector protein may be fused to the N terminus of the polypeptide or the C-terminus of the polypeptide. In some aspects, the polypeptide comprises a linker between the effector protein and the polypeptide. In some aspects, the linker comprises SEQ ID NO:314 or an amino acid having at least 80% sequence identity to SEQ ID NO:314. In some aspects, the linker has or has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:314. In some aspects, the polypeptide comprises one or more nuclear localization signals. In some aspects, the polypeptide comprises SEQ ID NO:317 or an amino acid sequence having at least 85% sequence identity to SEQ ID NO:317. In some aspects, the polypeptide comprises or comprises at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:317.

In some aspects, the target nucleic acid (nucleic acid that is to be modified) comprises a Protospacer Adjacent Motif (PAM) motif and wherein the adenine is at a position at least or at most 1, 2, 3, 4, 5, 6, 7, 8, 9, 10, 11, 12, 13, 14, or 15 (or any derivable range therein) bases distal from the PAM. In some aspects, the adenine is adjacent to a purine. In some aspects, the adenine is adjacent to a pyrimidine. In some aspects, the adenine base is modified to an inosine base. In some aspects, the adenine base is edited to a guanine base.

In some aspects, provided herein are polypeptides and methods that achieve at least about 95%, 96%, 97%, 98%, or 99% A-to-G conversion rates. In some embodiments, provided herein are methods that achieve at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or any range derivable therein, A-to-G conversion rates. In some aspects, provided herein are polypeptides and methods that achieve at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or any range derivable therein, A-to-G conversion rates, wherein the A is in the context of RA, wherein “R” represents a purine base. In some aspects, provided herein are polypeptides and methods that achieve at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or any range derivable therein, A-to-G conversion rates, wherein the A is in the context of YA, wherein “Y” represents a pyrimidine base.

In some aspects, the method is performed in vitro, in vivo, or ex vivo.

In aspects of the methods described herein, the method steps, such as steps (i)-(ix) are performed in the order that they are recited. In some aspects, step (i): generating a library of variant genes of the editor by mutagenesis comprises mutagenesis by chemical mutagens, error prone PCR, transposons, or DNA shuffling. In some aspects, the mutagenesis comprises mutagenesis by error prone PCR.

In some aspects, the library of comprises a combinatorial library with coverage of at least 80% coverage of the substitution combinations. The term “combinatorial library” refers to a library the comprises variants comprising different combinations of the substitutions. For example, a combinatorial library of 5 substitution variants of a gene would have 5⁵ variants when all possible combinations of the variants are covered (100% coverage). At 90% coverage, at least 90% of all possible combinations are represented. Thus, the combinatorial library may be a library that combines, combines at least, or combines at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 34, 35, 36, 37, 38, 39, 40, 41, 42, 43, 44, 45, 46, 47, 48, 49, or 50 substitutions, or any derivable range therein. In some aspects, the library provides or provides at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% coverage (or any derivable range therein) of all of the possible combinations. In some aspects, the library of comprises a combinatorial library with coverage of at least 95% coverage of the substitution combinations. In some aspects, the combinatorial library is created by overlapping PCR fragments comprising DNA encoding for the one or more substitutions. The library may comprise at least 1000 different editor variants. In some aspects, the library comprises, comprises at least, or comprises at most 100, 500, 1000, 1500, 2000, 2500, 3000, 3500, 4000, 4500, 5000, 6000, 7000, 8000, 9000, 10000, 11000, 12000, 13000, 14000, 15000, 16000, 17000, 18000, 19000, 20000, 25000, 30000, 35000, 40000, 45000, 50000, 60000, 70000, 80000, 90000, 100000, 120000, 140000, 160000, 180000, 200000, 250000, 300000, 400000, 500000, 600000, 700000, 800000, 900000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, or 1×10¹⁴, or any derivable range therein, different editor variants. In some aspects, the library comprises combinations of at least 3 of the one or more substitutions identified in the variants with increased fitness.

In some aspects, the editor comprises TadA, Cas9, Cas11, Cas12, Cas13, a zinc finger, Cpf1, CDA1, ADAR, ADAR1, ADAR2, a deaminase, an adenine base editor, a cytidine deaminase, APOBEC1, first-generation base editor (BE1), BE2, BE3, HF-BE3, BE4-GAM, YE1-BE3, EE-BE3, YE2-BE3, VQR-BE3, VRER-BE3, Sa-BE3, Sa-BE4, SaBE4-Gam, SaKKH-BE3, Cas12a-BE, Target-AID, Target-AID-NG, xBE3, eA3A-BE3, A3A-BE3, BE-PLUS, TAM, CRISPR-X, ABE7.9, ABE7.10, xABE, ABESa, VQR-ABE, VRQR-ABEs, VRER-ABE, SaKKH-ABE, Gam, an editor of SEQ ID NO:1-33, or a substitutional variant thereof. In one aspect, the editor comprises an adenine base editor. In one aspect, the editor comprises a cytidine deaminase. In some aspects, the editor comprises an adenine base editor or a cytidine deaminase. Editors are known in the art and described in, for example, Rees H A, Liu D R. Base editing: precision chemistry on the genome and transcriptome of living cells. Nat Rev Genet. 2018 December; 19(12):770-788. doi: 10.1038/s41576-018-0059-1. Erratum in: Nat Rev Genet. 2018 Oct. 19; PMID: 30323312; PMCID: PMC6535181, which is herein incorporated by reference for all purposes. In some aspects, the editor is an editor described in Rees H A, Liu D R. Base editing: precision chemistry on the genome and transcriptome of living cells. Nat Rev Genet. 2018 December; 19(12):770-788. doi: 10.1038/s41576-018-0059-1. Erratum in: Nat Rev Genet. 2018 Oct. 19; PMID: 30323312; PMCID: PMC6535181.

In some aspects, steps (ii), (v), and/or (viii), comprise selecting for one or more variants with increased fitness, wherein the selection comprises editing of at least two different nucleotides of a selection gene. The fitness refers to the variant's ability to confer survival to the cell, such as to the bacterial cell. For example, the fitness can be increased when editing is successful in a selection gene and confer survival to cells that express the selection gene under selective pressure. In a specific example, the library is transformed into bacterial cells and the bacterial cells are cultured under selection by an antibiotic. The bacterial cells may have an antibiotic resistance gene comprising mutations that require correction by the variant to make a functional protein. Variants with increased fitness will edit the antibiotic resistance gene to correct the mutations and confer antibiotic resistance to the cells. In some aspects, the selection gene comprises an antibiotic resistance gene. In some aspects, the increased fitness comprises an increase in the rate of deamination. In some aspects, the increased fitness comprises increased editing of an adenine in a RA context, wherein R denotes a purine base and A denotes an adenine. In some aspects, the increased fitness comprises increased editing of an adenine in a YA context, wherein Y denotes a pyrimidine base and A denotes an adenine. In some aspects, the increased fitness comprises increased editing at protospacer positions 1, 2, and/or 3.

In some aspects, the method further comprises cloning and/or sequencing the variants with increased fitness. In some aspects, the variants are sequenced by Next generation sequencing methods. Sequencing methods are known in the art and include, for example, massively parallel signature sequencing, polony sequencing, 454 pyrosequencing, illumine (Solexa) sequencing, SOLiD sequencing, Ion Torrent semiconductor sequencing, DNA nanoball sequencing, heliscope single molecule sequencing, single molecule real time sequencing, Sanger sequencing, and clone by clone sequencing.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-D a. Design for bacterial selection. b. A:T-to-G:C editing in HEK293T cells enabled by ABE-RAs at A4-A8 positions. Four genomic loci were assayed, with ABE7.10 as a control. c. A:T-to-G:C editing in HEK293T cells by ABE4.0-4.3, ABE5.0-5.2 versus ABE7.10, ABE8.20 and ABE8e at A4-A8 positions at five genomic loci. d. A:T-to-G:C editing in HEK293T cells by ABE4.0-4.3, ABE5.0-5.2 versus ABE7.10, ABE8.20 and ABE8e at A1-A3 positions at five genomic loci.

FIG. 2A-G. a. In vitro deamination assay for TadA8r, TadA8.20, and TadA8e. 5′-radiolabeled ssDNA oligos bearing a single GA or TA sequence were used as substrates. Left: PAGE gels of ssDNA oligos incubated with different deaminases followed by EndoV treatment. Top right: kapp of TadA8r, TadA8.20, and TadA8e on GA- or TA-containing probes. Bottom right: Fractions of deaminated DNA plotted as a function of time. Data were fitted using a nonlinear regression model in Graphpad. b. A:T-to-G:C editing in HEK293T cells by ABE7.10, ABE8.20, ABE8e, and ABE8r at A4-A8 positions at twelve genomic loci. c. A:T-to-G:C editing in HEK293T cells by ABE7.10, ABE8.20, ABE8e, and ABE8r at A1-A3 positions at twelve genomic loci. d. A:T-to-G:C editing in HEK293T cells by ABE7.10, ABE8.20, ABE8e, and ABE8r at A9-A14 positions at twelve genomic loci. e. A:T-to-G:C editing in HEK293T cells by ABE7.10, ABE8.20, ABE8e, and ABE8r at A1-A3 positions at additional eight genomic loci. f. Box plot for A:T-to-G:C editing in HEK293T cells by ABE7.10, ABE8.20, ABE8e, and ABE8r. Left: A1: n=6; A2: n=11; A3: n=11, lower and upper hinges represent first and third quartile; the center line represents the median; + represents mean; Right: A1 (RA): n=4; A1 (YA): n=2; A2 (RA): n=9; A2 (YA): n=2; A3 (RA): n=6; A3 (YA): n=5, lower and upper hinges represent first and third quartile; the center line represents the median; + represents mean. g. Box plot of A:T-to-G:C editing in HEK293T cells by ABE7.10, ABE8.20, ABE8e, and ABE8r grouped by sequence context and positions in protospacer. A1-A3 (RA): n=19; A1-A3 (YA): n=9; A4-A8 (RA): n=17; A4-A8 (YA): n=16; A9-A14 (RA): n=8; A9-A14 (YA): n=16, lower and upper hinges represent first and third quartile; the center line represents the median; + represents mean.

FIG. 3A-B. a. On- and off-target editing frequencies of ABE7.10, ABE8.20, ABE8e, and ABE8r. Three genomic sites were assayed. Left: the most strongly edited A in on-target sites and the most strongly edited A in off-target sites are plotted. ON means on-target editing; OT means off-target editing; Right: ratio of on-target to off-target editing. b. Cas9-independent off-target A:T-to-G:C editing detected by the orthogonal R-loop assay at each R-loop site created by dSaCas9 and a SaCas9 sgRNA.

FIG. 4A-D. a. A:T-to-G:C editing in HEK293T cells by VRQR-ABEs and NG-ABEs at A4-A8 position in protospacer. b. A:T-to-G:C editing in HEK293T cells by dSpABE7.10, dSpABE8.20, dSpABE8e and dSpABE8r at A4-A8 position in protospacers. c. A:T-to-G:C editing in HEK293T cells by SaABEs, SaKKH-ABEs, LbABEs and enAsABEs in the strong editing window. d. Box plot of A:T-to-G:C editing in HEK293T cells by SaABEs and SaKKH-ABEs based on sequence context. RA: n=8; YA: n=15, lower and upper hinges represent first and third quartiles, the center line represents the median, + represents mean.

FIG. 5A-B. a. Base-editing efficiency in HEK293T cells at two PCSK9 splicing sites by ABE7.10, ABE8.20, ABE8e, and ABE8r. A3 in site 50 and A3 in site 51 are the PCSK9 splicing sites. b. Correcting a G:C-to-A:T mutation in ABCA4 by ABE8r with two different sgRNAs. A6 in site 52 and A3 in site 53 are the target As.

FIG. 6A-C. Directed evolution of TadA to function on deoxyadenosine in “RA” sequences. a. Methylation of “GATC” sequences in E. coli. Two restriction enzymes, DpnI and DpnII, are employed to confirm methylation of the target “GATC” in the chloramphenicol acetyl transferase gene. b. Unmethylated and methylated E. coli tRNA^{M} (ACG) treated with wildtype TadA and TadA71.10. Unmethylated and methylated tRNA were prepared through in vitro transcription using ATP and N⁶-methyl-ATP as starting materials, respectively. Treated RNA was reverse transcribed, amplified by PCR, and subjected to Sanger sequencing. c. Serial dilutions of E. coli transformed with the selection plasmid and denoted editor plasmids plated on 0, 16, or 32 μg/mL chloramphenicol. Two individual colonies from each transformation were assayed. FIG. 6B shows sequences: GCAUCCGUAGCUCAGCUGGAUAGAGUACUCGGCUACGAACCGAGCGGUCGGAG GUUCGAAUCCUCCCGGAUGCACCA (SEQ ID NO:125); GUACUCGGCUACGAACCAG (SEQ ID NO:279); and GUACUCGGCUACGAACCGAG (SEQ ID NO:280);

FIG. 7A-B. Initial-round directed evolution for TadA. a. Mutations identified in colonies that passed selection and validation. b. Serial dilutions of E. coli transformed with the selection plasmid and denoted editor plasmids plated on 0, 64, or 128 μg/mL chloramphenicol. Two individual colonies from each transformation were assayed.

FIG. 8A-B. Second-round directed evolution for TadA. a. Mutations identified in colonies that passed selection and validation. b. Serial dilutions of E. coli transformed with the selection plasmid and denoted editor plasmids plated on 0, 25, or 50 μg/mL kanamycin.

FIG. 9A-B. Third-round directed evolution for TadA. a. Mutations identified in colonies that passed selection and validation. b. Serial dilutions of E. coli transformed with the selection plasmid and denoted editor plasmids plated on 0, 400, or 800 μg/mL kanamycin.

FIG. 10. A:T-to-G:C editing in HEK293T cells enabled by ABE-RA1.0, 1.1, 2.0, 2.1, 3.0, 3.1, 3.2, and 3.3. Four target sites were assayed, with ABE7.10 as a control.

FIG. 11A-B. Fourth-round directed evolution for TadA. a. Mutations identified in colonies that passed selection and validation. b. Serial dilutions of E. coli transformed with the selection plasmid and denoted editor plasmids plated on 0, 400, or 800 μg/mL kanamycin.

FIG. 12. Mutations in colonies harvested in fifth-round directed evolution.

FIG. 13A-C. A:T-to-G:C editing in HEK293T cells enabled by ABE-RA4s, ABE-RA5s. Five target sites were assayed, with ABE7.10, ABE8.20, ABE8e as controls.

FIG. 14. A:T-to-G:C editing on N6-methyldeoxyadenosine in a plasmid in HEK293T cells and genomic site containing GATC sequence in HEK293T cells enabled by ABE7.10, ABE8.20, ABE-RA1.0, ABE-RA1.1 and ABE-RA2.0.

FIG. 15A-B. A:T-to-G:C editing in HEK293T cells by ABE7.10, ABE8.20, ABE8e, and ABE8r in the entire protospacer for twelve sites.

FIG. 16. Indel frequencies observed with ABE7.10, ABE8.20, ABE8e, and ABE8r at twelve sites.

FIG. 17A-B. A:T-to-G:C editing in HEK293T cells by ABE7.10, ABE8.20, ABE8e, and ABE8r in the entire protospacer for additional eight sites.

FIG. 18A-C. On-target and Cas9-dependent off-target editing generated by ABE7.10, ABE8.20, ABE8e, and ABE8r. Three target sites were chosen with 2-4 off-target sites evaluated for each target site.

FIG. 19. On-target editing enforced by ABEs at site 1 for orthogonal R-loop assays

FIG. 20. Cas9-independent off-target A⋅T-to-G⋅C editing detected by the orthogonal R-loop assay.

FIG. 21. A:T-to-G:C editing in HEK293T cells by VRQR-ABE7.10, VRQR-ABE8.20, VRQR-ABE8e, and VRQR-ABE8r. Four genomic loci were tested.

FIG. 22. A:T-to-G:C editing in HEK293T cells by NG-ABE7.10, NG-ABE8.20, NG-ABE8e, and NG-ABE8r. Five genomic loci were tested.

FIG. 23. A:T-to-G:C editing in HEK293T cells by NRCH-ABEs, and NRTH-ABEs.

FIG. 24. A:T-to-G:C editing in HEK293T cells by dSpABE7.10, dSpABE8.20, dSpABE8e, and dSpABE8r at 6 genomic loci.

FIG. 25. Indel frequencies detected for dSpABE7.10, dSpABE8.20, dSpABE8e, and dSpABE8r at seven targets sites in HEK293T cells by.

FIG. 26. A:T-to-G:C editing in HEK293T cells by SaABE7.10, SaABE8.20, SaABE8e, and SaABE8r. Six genomic loci were tested.

FIG. 27. A:T-to-G:C editing in HEK293T cells by SaKKH-ABEs. Four genomic sites were tested.

FIG. 28A-B. a. A:T-to-G:C editing in HEK293T cells by LbABEs. b. A:T-to-G:C editing in HEK293T cells by enAsABEs.

### DETAILED DESCRIPTION OF THE INVENTION

### I. Proteinaceous Compositions

As used herein, a “protein” “peptide” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more contiguous amino acids or nucleic acids, or any range derivable therein, of SEQ ID NOS:1-33. In specific aspects, the peptide or polypeptide is or is based on a human sequence. In certain aspects, the peptide or polypeptide is not naturally occurring and/or is in a combination of peptides or polypeptides.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 substitutions (or any range derivable therein).

In some aspects, the polypeptide comprises one or more substitutions at one or more amino acid positions selected from amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and/or 200 of any of SEQ ID NOS:1-33, wherein each substitution is independently chosen from an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine; and wherein the polypeptide is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOS:1-33.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) of SEQ ID NOS:1-33.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) of SEQ ID NOS:1-33 and have or have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOS:1-33.

In some aspects, the protein, polypeptide, or nucleic acid may comprise, comprise at least, or comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) contiguous amino acids or nucleic acids of SEQ ID NOS:1-33.

In some aspects, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) contiguous amino acids of SEQ ID NOS:1-33 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOS:1-33.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 of any of SEQ ID NOS:1-33 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) contiguous amino acids or nucleotides of any of SEQ ID NOS:1-33.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5:1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein structure.

In some aspects of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### II. Nucleic Acids

In certain aspects, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one aspect, one or more particular amino acid residues are changed using, for example, a site-directed mutagenesis protocol. In another aspect, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another aspect, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred aspect, the nucleic acid molecules are oligonucleotides. In a more preferred aspect, the oligonucleotides are from highly variable regions of the heavy and light or alpha and beta chains of the antibody or TCR of interest. In an even more preferred aspect, the oligonucleotides encode all or part of one or more of the CDRs or TCRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### III. Polypeptide Expression

In some aspects, there are nucleic acid molecule encoding polypeptides or peptides of the disclosure (e.g TCR genes). These may be generated by methods known in the art, e.g., isolated from B cells of mice that have been immunized and isolated, phage display, expressed in any suitable recombinant expression system and allowed to assemble to form antibody molecules or by recombinant methods.

### A. Expression

The nucleic acid molecules may be used to express large quantities of polypeptides. If the nucleic acid molecules are derived from a non-human, non-transgenic animal, the nucleic acid molecules may be used for humanization of the TCR genes.

### B. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, alpha chain, beta chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin or TCR sequence with appropriate restriction sites engineered so that any variable region sequences can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### C. Expression Systems

Numerous expression systems exist that comprise at least a part or all of the expression vectors discussed above. Prokaryote- and/or eukaryote-based systems can be employed for use with an aspect to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### IV. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### A. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy, light, alpha, and beta chains of an antibody or TCR, or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### V. Kits

The present disclosure additionally provides kits for modifying and/or detecting modified adenosines in a target DNA. Each kit may also include additional components that are useful for amplifying the nucleic acid, or sequencing the nucleic acid, or other applications of the present disclosure as described herein. The kit may optionally provide additional components that are useful in the procedure. These optional components include buffers, capture reagents, developing reagents, labels, reacting surfaces, means for detection, control samples, instructions, and interpretive information. The kit may also include reagents for DNA isolation and/or purification.

### VI. Sequences

| | | SEQ |
|---|---|---|
| | | ID |
| Description | Sequence | NO: |
| WT | MSEVEFSHEYWMRHALTLAKRAWDEREVPVGAVLVHNNRVIG | 1 |
| | EGWNRPIGRHDPTAHAEIMALRQGGLVMQNYRLIDATLYVTLE | |
| | PCVMCAGAMIHSRIGRVVFGARDAKTGAAGSLMDVLHHPGMN | |
| | HRVEITEGILADECAALLSDFFRMRRQEIKAQKKAQSSTD | |
| TadA7.10 | MSEVEFSHEYWMRHALTLAKRARDEREVPVGAVLVLNNRVIGE | 31 |
| | GWNRAIGLHDPTAHAEIMALRQGGLVMQNYRLIDATLYVTFEP | |
| | CVMCAGAMIHSRIGRVVFGVRNAKTGAAGSLMDVLHYPGMNH | |
| | RVEITEGILADECAALLCYFFRMPRQVFNAQKKAQSSTD | |
| TadA8.20 | MSEVEFSHEYWMRHALTLAKRARDEREVPVGAVLVLNNRVIGE | 32 |
| | GWNRAIGLHDPTAHAEIMALRQGGLVMQNYRLYDATLYSTFEP | |
| | CVMCAGAMIHSRIGRVVFGVRNAKTGAAGSLMDVLHHPGMNH | |
| | RVEITEGILADECAALLCRFFRMPRRVFNAQKKAQSSTD | |
| TadA8e | MSEVEFSHEYWMRHALTLAKRARDEREVPVGAVLVLNNRVIGE | 33 |
| | GWNRAIGLHDPTAHAEIMALRQGGLVMQNYRLIDATLYVTFEP | |
| | CVMCAGAMIHSRIGRVVFGVRNSKRGAAGSLMNVLNYPGMNH | |
| | RVEITEGILADECAALLCDFYRMPRQVFNAQKKAQSSIN | |
| TadA-R1.0 | MSEVEFSHEYWMRHALTLAKRAWDEREVPVGAVLVHNNRVIG | 2 |
| (pyx0331) | EGWNRPIGRHDPTAHAEIMALRQGGLVMQNYRLIDATLYVTLE | |
| | PCVMCAGAMIHSRIGRVVFGARGAKTGAAGSLMDVLHHPGMN | |
| | HRVEITEGILADECAALLSDFFRMRRQEIKAQKKAQSSTD | |
| TadA-R1.1 | MSEVEFSHEYWMRHALTLAKRAWDEREVPVGAVLVHNNRVIG | 3 |
| (pyx047a) | EGWNRPIGRHDPTAHAEIMALRQGGLVMQNYRLIDATLYVTLE | |
| | PCVMCAGAMIHSRIGRVVFGARGAKTGAAGSLMDVLHHPGMN | |
| | HRVEITEGILADECAALLSDFFRMRRQEIKAQKNAQSSTD | |
| TadA-R2.0 | MSEVEFSHEYWMRHALTLAKRAWDEREVPVGAVLVHNNRVIG | 16 |
| | EGWNRAIGHHDPTAHAEIMALRQGGLVMQNYRLFDATLYVTL | |
| | EPCVMCAGAMIHSRIGRVVFGARGARTGAAGSLMDVLRHPGIK | |
| | HRVEITEGILADECAALLSDFFRMRRQEIKAQKNAQSSTD | |
| TadA-R2.1 | MSEVEFSHEYWMRHALTLAKRAWDEREVPVGAVLVHNNRVIG | 17 |
| | EGWNRAIGHHDPTAHAEIMALRQGGLVMQNYRLIDATLYVTLE | |
| | PCVMCAGAMIHSRIGRVVFGARGARTGAAGSLMDVLRHPGIKH | |
| | RVEITEGILADECAALLSDFFRMRRQEIKAQKKAQSSTD | |
| TadA-R3.0 | MSEVEFSHEYWMRHALTLAKRAWDERDVPVGAVLVHNNRVIG | 18 |
| | EGWNRAIGHHDPTAHAEIMALRQGGLVMQNYRLFDATLYVTL | |
| | EPCVMCAGAMIHSRIGRVVFGARGARTGAAGSLMDVLRHPGIK | |
| | HRVEITEGILADECAALLSDFFRMRRQEIKAQKNAQSSTD | |
| TadA-R3.1 | MSEVEFSHEYWMRHALTLAKRAWDERDVPVGAVLVHNNRVIG | 19 |
| | EGWNKAIGHHDPTAHAEIMALRQGGLVMQNYRLFDATLYVTL | |
| | EPCVMCAGAMIHSRIGRVVFGARGARTGAAGSLMDVLRHPGIK | |
| | HRVEITEGILADECAALLSDFFRMRRQEIKAQKNAQSSTD | |
| TadA-R3.2 | MSEVEFSHEYWMRHALTLAKRAWDERDVPVGAVLVHNNRVIG | 20 |
| | EGWNRAIGHHDPTAHAEIMALRQGGLVMQNYRLIDATLYVTLE | |
| | PCVMCAGAMIHSRIGRVVFGARGARTGAVGSLMDVLRHPGIKH | |
| | RVEITEGILADECAALLSDFFRMRRQEIKAQKKAQSSTD | |
| TadA-R3.3 | MSEVEFSHEYWMRHALTLAKRAWDERDVPVGAVLVHNNRVIG | 21 |
| | EGWNKAIGHHDPTAHAEIMALRQGGLVMQNYRLFDATLYVTL | |
| | EPCVMCAGAMIHSRIGRVVFGARGARTGAVGSLMDVLRHPGIK | |
| | HRVEITEGILADECAALLSDFFRMRRQEIKAQKKAQSSTD | |
| TadA-R4.0 | MSEVEFSHEYWMRHALTLAKRAWDEREVPVGAVLVHNNRVIG | 11 |
| (088a) | EGWNRAIGHHDPTAHAEIMALRQGGLVMQNYRLFDATLYVTL | |
| | EPCVMCAGAMIHSRIGRVVFGVRGARHGAAGSLMNVLHYPGIK | |
| | HRVEITEGILADECAALLSRFFRMPRRVFKAQKKAQSSTD | |
| TadA-R4.1 | MSEVEFSHEYWMRHALTLAKRAWDEREVPVGAVLVHNNRVIG | 22 |
| | EGWNRAIGHHDPTAHAEIMALRQGGLVMQNYRLFDATLYVTL | |
| | EPCVMCAGAMIHSRIGRVVFGVRGARHGAAGSLMNVLRYPGIK | |
| | HRVEITEGILADECAALLSRFFRMPRRVFKAQKKAQSSTD | |
| TadA-R4.2 | MSEVEFSHEYWMRHALTLAKRAWDEREVPVGAVLVHNNRVIG | 12 |
| (088c) | EGWNRAIGHHDPTAHAEIMALRQGGLVMQNYRLFDATLYVTL | |
| | EPCVMCAGAMIHSRIGRVVFGVRGARHGAAGSLMNVLHYPGIK | |
| | HRVEITEGILADECAALLCRFFRMPRRVFKAQKKAQSSTD | |
| TadA-R4.3 | MSEVEFSHEYWMRHALTLAKRAWDEREVPVGAVLVHNNRVIG | 13 |
| (088d) | EGWNRAIGHHDPTAHAEIMALRQGGLVMQNYRLFDATLYVTL | |
| | EPCVMCAGAMIHSRIGRVVFGVRGARHGAAGSLMNVLHYPGIK | |
| | HRVEITEGILADECAALLSRFFRMPRRVFNAQKKAQSSTD | |
| TadA-R4.4 | MSEVEFSHEYWMRHALTLAKRAWDEREVPVGAVLVHNNRVIG | 14 |
| 088e) | EGWNRAIGHHDPTAHAEIMALRQGGLVMQNYRLFDATLYVTL | |
| | EPCVMCAGAMIHSRIGRVVFGVRGARHGAAGSLMNVLHYPGIK | |
| | HRVEITEGILADECAALLSRFFRMPRRVFKAQKNAQSSTD | |
| TadA-R4.5 | MSEVEFSHEYWMRHALTLAKRAWDEREVPVGAVLVHNNRVIG | 15 |
| (088f) | EGWNRAIGHHDPTAHAEIMALRQGGLVMQNYRLFDATLYVTL | |
| | EPCVMCAGAMIHSRIGRVVFGVRGARHGAAGSLMNVLHYPGIK | |
| | HRVEITEGILADECAALLSRFFRMPRRVFKAQKKAQSSID | |
| TadA-R4.6 | MSEVEFSHEYWMRHALTLAKRAWDEREVPVGAVLVHNNRVIG | 23 |
| | EGWNRAIGHHDPTAHAEIMALRQGGLVMQNYRLFDATLYVTL | |
| | EPCVMCAGAMIHSRIGRVVFGVRGARHGAAGSLMNVLHYPGIK | |
| | HRVEITEGILADECAALLSRFFRMPRRVFKAQKKAQSSTN | |
| TadA-R5.0 | MSEVEFSHEYWMRHALTLAKRARDEREVPVGAVLVLNNRVIGE | 24 |
| | GWNKAIGLHDPTAHAEIMALRQGGLVMQNYRLFDATLYSTLEP | |
| | CVMCAGAMIHSRIGRVVFGVRGARHGAAGSLMNVLHYPGIKH | |
| | RVEITEGILADECAALLCRFFRMPRRVFKAQKKAQSSTD | |
| TadA-R5.1 | MSEVEFSHEYWMRHALTLAKRARDEREVPVGAVLVLNNRVIGE | 25 |
| | GWNKAIGLHDPTAHAEIMALRQGGLVMQNYRLFDATLYSTLEP | |
| | CVMCAGAMIHSRIGRVVFGVRGARHGAAGSLMNVLNYPGIKH | |
| | RVEITEGILADECAALLCRFFRMPRRVFKAQKKAQSSTD | |
| TadA-R5.2 | MSEVEFSHEYWMRHALTLAKRARDEREVPVGAVLVLNNRVIGE | 26 |
| | GWNKAIGLHDPTAHAEIMALRQGGLVMQNYRLYDATLYSTLEP | |
| | CVMCAGAMIHSRIGRVVFGVRGARHGAVGSLMNVLHYPGIKH | |
| | RVEITEGILADECAALLCRFFRMPRRVFKAQKKAQSSTD | |
| TadA-R5.3 | MSEVEFSHEYWMRHALTLAKRARDEREVPVGAVLVLNNRVIGE | 27 |
| | GWNKAIGLHDPTAHAEIMALRQGGLVMQNYRLFDATLYSTLEP | |
| | CVMCAGAMIHSRIGRVVFGVRGSRHGAVGSLMNVLHYPGIKHR | |
| | VEITEGILADECAALLSRFFRMPRRVFKAQKKAQSSTD | |
| TadA-R5.4 | MSEVEFSHEYWMRHALTLAKRARDEREVPVGAVLVHNNRVIG | 28 |
| | EGWNKAIGLHDPTAHAEIMALRQGGLVMQNYRLYDATLYSTLE | |
| | PCVMCAGAMIHSRIGRVVFGVRGSRHGAVGSLMNVLHYPGIKH | |
| | RVEITEGILADECAALLSRFFRMPRRVFKAQKKAQSSTD | |
| TadA-R5.5 | MSEVEFSHEYWMRHALTLAKRARDEREVPVGAVLVLNNRVIGE | 29 |
| | GWNKAIGLHDPTAHAEIMALRQGGLVMQNYRLYDATLYSTLEP | |
| | CVMCAGAMIHSRIGRVVFGVRGARHGAVGSLMNVLHYPGIKH | |
| | RVEITEGILADECAALLCRFFRMPRRVFNAQKNAQSSIN | |
| TadA-R5.6 | MSEVEFSHEYWMRHALTLAKRARDEREVPVGAVLVLNNRVIGE | 30 |
| | GWNKAIGLHDPTAHAEIMALRQGGLVMQNYRLYDATLYSTLEP | |
| | CVMCAGAMIHSRIGRVVFGVRGARHGAVGSLMNVLHYPGIKH | |
| | RVEITEGILADECAALLCRFFRMPRRVFKAQKKAQSSIN | |
| pyx047c | MSEVEFSHEYWMRHALTLAKRAWDEREVPVGAVLVHNNRVIG | 4 |
| | EGWNRAIGRHDPTAHAEIMALRQGGLVMQNYRLIDATLYVTLE | |
| | PCVMCAGAMIHSRIGRVVFGARGAKTGAAGSLMDVLHHPGMN | |
| | HRVEITEGILADECAALLSDFFRMRRQEIKAQKNAQSSTD | |
| pyx047d | MSEVEFSHEYWMRHALTLAKRAWDEREVPVGAVLVHNNRVIG | 5 |
| | EGWNRAIGRHDPTAHAEIMALRQGGLVMQNYRLFDATLYVTLE | |
| | PCVMCAGAMIHSRIGRVVFGARGAKTGAAGSLMDVLHHPGMN | |
| | HRVEITEGILADECAALLSDFFRMRRQEIKAQKNAQSSTD | |
| pyx047e | MSEVEFSHEYWMRHALTLAKRAWDEREVPVGAVLVHNNRVIG | 6 |
| | EGWNRAIGRHDPTAHAEIMALRQGGLVMQNYRLIDATLYVTLE | |
| | PCVMCAGAMIHSRIGRVVFGARGAKTGAAGSLMDVLHHPGINH | |
| | RVEITEGILADECAALLSDFFRMRRQEIKAQKNAQSSTD | |
| pyx047f | MSEVEFSHEYWMRHALTLAKRAWDEREVPVGAVLVHNNRVIG | 7 |
| | EGWNRAIGRHDPTAHAEIMALRQGGLVMQNYRLIDATLYVTLE | |
| | PCVMCAGAMIHSRIGRVVFGARGAKTGAAGSLMDVLHHPGMK | |
| | HRVEITEGILADECAALLSDFFRMRRQEIKAQKNAQSSTD | |
| pyx047g | MSEVEFSHEYWMRHALTLAKRAWDEREVPVGAVLVHNNRVIG | 8 |
| | EGWNRAIGRHDPTAHAEIMALRQGGLVMQNYRLFDATLYVTLE | |
| | PCVMCAGAMIHSRIGRVVFGARGARTGAAGSLMDVLHHPGMK | |
| | HRVEITEGILADECAALLSDFFRMRRQEIKAQKNAQSSTD | |
| pyx047i | MSEVEFSHEYWMRHALTLAKRAWDEREVPVGAVLVHNNR VIG | 9 |
| | EGWNRAIGHHDPTAHAEIMALRQGGLVMQNYRLFDATLYVTL | |
| | EPCVMCAGAMIHSRIGRVVFGARGARTGAAGSLMDVLHHPGIK | |
| | HRVEITEGILADECAALLSDFFRMRRQEIKAQKNAQSSTD | |
| pyx047k | MSEVEFSHEYWMRHALTLAKRAWDEREVPVGAVLVHNNRVIG | 10 |
| | EGWNRPIGRHDPTAHAEIMALRQGGLVMQNYRLIDATLYVTLE | |
| | PCVMCAGAMIHSRIGRVVFGARGARTGAAGSLMDVLHHPGMK | |
| | HRVEITEGILADECAALLSDFFRMRRQEIKAQKNAQSSTD | |
| TadA-R1.0 | SEVEFSHEYWMRHALTLAKRAWDEREVPVGAVLVHNNRVIGE | 291 |
| (pyx0331)-x | GWNRPIGRHDPTAHAEIMALRQGGLVMQNYRLIDATLYVTLEP | |
| | CVMCAGAMIHSRIGRVVFGARGAKTGAAGSLMDVLHHPGMNH | |
| | RVEITEGILADECAALLSDFFRMRRQEIKAQKKAQSSTD | |
| TadA-R1.1 | SEVEFSHEYWMRHALTLAKRAWDEREVPVGAVLVHNNRVIGE | 292 |
| (pyx047a)-x | GWNRPIGRHDPTAHAEIMALRQGGLVMQNYRLIDATLYVTLEP | |
| | CVMCAGAMIHSRIGRVVFGARGAKTGAAGSLMDVLHHPGMNH | |
| | RVEITEGILADECAALLSDFFRMRRQEIKAQKNAQSSTD | |
| TadA-R2.0- | SEVEFSHEYWMRHALTLAKRAWDEREVPVGAVLVHNNRVIGE | 293 |
| x | GWNRAIGHHDPTAHAEIMALRQGGLVMQNYRLFDATLYVTLEP | |
| | CVMCAGAMIHSRIGRVVFGARGARTGAAGSLMDVLRHPGIKHR | |
| | VEITEGILADECAALLSDFFRMRRQEIKAQKNAQSSTD | |
| TadA-R2.1- | SEVEFSHEYWMRHALTLAKRAWDEREVPVGAVLVHNNRVIGE | 294 |
| x | GWNRAIGHHDPTAHAEIMALRQGGLVMQNYRLIDATLYVTLEP | |
| | CVMCAGAMIHSRIGRVVFGARGARTGAAGSLMDVLRHPGIKHR | |
| | VEITEGILADECAALLSDFFRMRRQEIKAQKKAQSSTD | |
| TadA-R3.0- | SEVEFSHEYWMRHALTLAKRAWDERDVPVGAVLVHNNRVIGE | 295 |
| x | GWNRAIGHHDPTAHAEIMALRQGGLVMQNYRLFDATLYVTLEP | |
| | CVMCAGAMIHSRIGRVVFGARGARTGAAGSLMDVLRHPGIKHR | |
| | VEITEGILADECAALLSDFFRMRRQEIKAQKNAQSSTD | |
| TadA-R3.1- | SEVEFSHEYWMRHALTLAKRAWDERDVPVGAVLVHNNRVIGE | 296 |
| x | GWNKAIGHHDPTAHAEIMALRQGGLVMQNYRLFDATLYVTLE | |
| | PCVMCAGAMIHSRIGRVVFGARGARTGAAGSLMDVLRHPGIKH | |
| | RVEITEGILADECAALLSDFFRMRRQEIKAQKNAQSSTD | |
| TadA-R3.2- | SEVEFSHEYWMRHALTLAKRAWDERDVPVGAVLVHNNRVIGE | 297 |
| x | GWNRAIGHHDPTAHAEIMALRQGGLVMQNYRLIDATLYVTLEP | |
| | CVMCAGAMIHSRIGRVVFGARGARTGAVGSLMDVLRHPGIKHR | |
| | VEITEGILADECAALLSDFFRMRRQEIKAQKKAQSSTD | |
| TadA-R3.3- | SEVEFSHEYWMRHALTLAKRAWDERDVPVGAVLVHNNRVIGE | 298 |
| x | GWNKAIGHHDPTAHAEIMALRQGGLVMQNYRLFDATLYVTLE | |
| | PCVMCAGAMIHSRIGRVVFGARGARTGAVGSLMDVLRHPGIKH | |
| | RVEITEGILADECAALLSDFFRMRRQEIKAQKKAQSSTD | |
| TadA-R4.0 | SEVEFSHEYWMRHALTLAKRAWDEREVPVGAVLVHNNRVIGE | 299 |
| (088a)-x | GWNRAIGHHDPTAHAEIMALRQGGLVMQNYRLFDATLYVTLEP | |
| | CVMCAGAMIHSRIGRVVFGVRGARHGAAGSLMNVLHYPGIKH | |
| | RVEITEGILADECAALLSRFFRMPRRVFKAQKKAQSSTD | |
| TadA-R4.1- | SEVEFSHEYWMRHALTLAKRAWDEREVPVGAVLVHNNRVIGE | 300 |
| x | GWNRAIGHHDPTAHAEIMALRQGGLVMQNYRLFDATLYVTLEP | |
| | CVMCAGAMIHSRIGRVVFGVRGARHGAAGSLMNVLRYPGIKHR | |
| | VEITEGILADECAALLSRFFRMPRRVFKAQKKAQSSTD | |
| TadA-R4.2 | SEVEFSHEYWMRHALTLAKRAWDEREVPVGAVLVHNNRVIGE | 301 |
| (088c)-x | GWNRAIGHHDPTAHAEIMALRQGGLVMQNYRLFDATLYVTLEP | |
| | CVMCAGAMIHSRIGRVVFGVRGARHGAAGSLMNVLHYPGIKH | |
| | RVEITEGILADECAALLCRFFRMPRRVFKAQKKAQSSTD | |
| TadA-R4.3 | SEVEFSHEYWMRHALTLAKRAWDEREVPVGAVLVHNNRVIGE | 302 |
| (088d)-x | GWNRAIGHHDPTAHAEIMALRQGGLVMQNYRLFDATLYVTLEP | |
| | CVMCAGAMIHSRIGRVVFGVRGARHGAAGSLMNVLHYPGIKH | |
| | RVEITEGILADECAALLSRFFRMPRRVFNAQKKAQSSTD | |
| TadA-R4.4 | SEVEFSHEYWMRHALTLAKRAWDEREVPVGAVLVHNNRVIGE | 303 |
| 088e)-x | GWNRAIGHHDPTAHAEIMALRQGGLVMQNYRLFDATLYVTLEP | |
| | CVMCAGAMIHSRIGRVVFGVRGARHGAAGSLMNVLHYPGIKH | |
| | RVEITEGILADECAALLSRFFRMPRRVFKAQKNAQSSTD | |
| TadA-R4.5 | SEVEFSHEYWMRHALTLAKRAWDEREVPVGAVLVHNNRVIGE | 304 |
| (088f)-x | GWNRAIGHHDPTAHAEIMALRQGGLVMQNYRLFDATLYVTLEP | |
| | CVMCAGAMIHSRIGRVVFGVRGARHGAAGSLMNVLHYPGIKH | |
| | RVEITEGILADECAALLSRFFRMPRRVFKAQKKAQSSID | |
| TadA-R4.6- | SEVEFSHEYWMRHALTLAKRAWDEREVPVGAVLVHNNRVIGE | 305 |
| x | GWNRAIGHHDPTAHAEIMALRQGGLVMQNYRLFDATLYVTLEP | |
| | CVMCAGAMIHSRIGRVVFGVRGARHGAAGSLMNVLHYPGIKH | |
| | RVEITEGILADECAALLSRFFRMPRRVFKAQKKAQSSTN | |
| TadA-R5.0- | SEVEFSHEYWMRHALTLAKRARDEREVPVGAVLVLNNRVIGEG | 306 |
| x | WNKAIGLHDPTAHAEIMALRQGGLVMQNYRLFDATLYSTLEPC | |
| | VMCAGAIHSRIGRVVFGVRGARHGAAGSLMNVLHYPGIKHRVE | |
| | ITEGILADECAALLCRFFRMPRRVFKAQKKAQSSTD | |
| TadA-R5.1- | SEVEFSHEYWMRHALTLAKRARDEREVPVGAVLVLNNRVIGEG | 307 |
| x | WNKAIGLHDPTAHAEIMALRQGGLVMQNYRLFDATLYSTLEPC | |
| | VMCAGAMIHSRIGRVVFGVRGARHGAAGSLMNVLNYPGIKHR | |
| | VEITEGILADECAALLCRFFRMPRRVFKAQKKAQSSTD | |
| TadA-R5.2- | SEVEFSHEYWMRHALTLAKRARDEREVPVGAVLVLNNRVIGEG | 308 |
| x | WNKAIGLHDPTAHAEIMALRQGGLVMQNYRLYDATLYSTLEPC | |
| | VMCAGAMIHSRIGRVVFGVRGARHGAVGSLMNVLHYPGIKHR | |
| | VEITEGILADECAALLCRFFRMPRRVFKAQKKAQSSTD | |
| TadA-R5.3- | SEVEFSHEYWMRHALTLAKRARDEREVPVGAVLVLNNRVIGEG | 309 |
| x | WNKAIGLHDPTAHAEIMALRQGGLVMQNYRLFDATLYSTLEPC | |
| | VMCAGAMIHSRIGRVVFGVRGSRHGAVGSLMNVLHYPGIKHRV | |
| | EITEGILADECAALLSRFFRMPRRVFKAQKKAQSSTD | |
| TadA-R5.4- | SEVEFSHEYWMRHALTLAKRARDEREVPVGAVLVHNNRVIGEG | 310 |
| x | WNKAIGLHDPTAHAEIMALRQGGLVMQNYRLYDATLYSTLEPC | |
| | VMCAGAMIHSRIGRVVFGVRGSRHGAVGSLMNVLHYPGIKHRV | |
| | EITEGILADECAALLSRFFRMPRRVFKAQKKAQSSTD | |
| TadA-R5.5- | SEVEFSHEYWMRHALTLAKRARDEREVPVGAVLVLNNRVIGEG | 311 |
| x | WNKAIGLHDPTAHAEIMALRQGGLVMQNYRLYDATLYSTLEPC | |
| | VMCAGAMIHSRIGRVVFGVRGARHGAVGSLMNVLHYPGIKHR | |
| | VEITEGILADECAALLCRFFRMPRRVFNAQKNAQSSIN | |
| TadA-R5.6- | SEVEFSHEYWMRHALTLAKRARDEREVPVGAVLVLNNRVIGEG | 312 |
| x | WNKAIGLHDPTAHAEIMALRQGGLVMQNYRLYDATLYSTLEPC | |
| | VMCAGAMIHSRIGRVVFGVRGARHGAVGSLMNVLHYPGIKHR | |
| | VEITEGILADECAALLCRFFRMPRRVFKAQKKAQSSIN | |

| | | SEQ |
|---|---|---|
| | | ID |
| Effector | Sequence | NO |
| SpCas9 | DKKYSIGLAIGTNSVGWAVITDEYKVPSKKFKVLGNTDRHSIKKNLIGA | 281 |
| nickase | LLFDSGETAEATRLKRTARRRYTRRKNRICYLQEIFSNEMAKVDDSFFH | |
| | RLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFE | |
| | ENPINASGVDAKAILSARLSKSRRLENLIAQLPGEKKNGLFGNLIALSLG | |
| | LTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPE | |
| | KYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILT | |
| | FRIPYYVGPLARGNSRFAWMTRKSEETITPWNFEEVVDKGASAQSFIER | |
| | MTNFDKNLPNEKVLPKHSLLYEYFTVYNELTKVKYVTEGMRKPAFLSG | |
| | EQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFA | |
| | NRNFMQLIHDDSLTFKEDIQKAQVSGQGDSLHEHIANLAGSPAIKKGIL | |
| | QTVKVVDELVKVMGRHKPENIVIEMARENQTTQKGQKNSRERMKRIE | |
| | EGIKELGSQILKEHPVENTQLQNEKLYLYYLQNGRDMYVDQELDINRLS | |
| | DYDVDHIVPQSFLKDDSIDNKVLTRSDKNRGKSDNVPSEEVVKKMKNY | |
| | WRQLLNAKLITQRKFDNLTKAERGGLSELDKAGFIKRQLVETRQITKHV | |
| | AQILDSRMNTKYDENDKLIREVKVITLKSKLVSDFRKDFQFYKVREINN | |
| | YHHAHDAYLNAVVGTALIKKYPKLESEFVYGDYKVYDVRKMIAKSEQ | |
| | EIGKATAKYFFYSNIMNFFKTEITLANGEIRKRPLIETNGETGEIVWDKG | |
| | RDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSS | |
| | FEKNPIDFLEAKGYKEVKKDLIIKLPKYSLFELENGRKRMLASAGELQK | |
| | GNELALPSKYVNFLYLASHYEKLKGSPEDNEQKQLFVEQHKHYLDEIIE | |
| | QISEFSKRVILADANLDKVLSAYNKHRDKPIREQAENIIHLFTLTNLGAP | |
| | AAFKYFDTTIDRKRYTSTKEVLDATLIHQSITGLYETRIDLSQLGGD | |
| SpCas9- | DKKYSIGLAIGTNSVGWAVITDEYKVPSKKFKVLGNTDRHSIKKNLIGA | 282 |
| VRQR | LLFDSGETAEATRLKRTARRRYTRRKNRICYLQEIFSNEMAKVDDSFFH | |
| | RLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFE | |
| | ENPINASGVDAKAILSARLSKSRRLENLIAQLPGEKKNGLFGNLIALSLG | |
| | LTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPE | |
| | KYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILT | |
| | FRIPYYVGPLARGNSRFAWMTRKSEETITPWNFEEVVDKGASAQSFIER | |
| | MTNFDKNLPNEKVLPKHSLLYEYFTVYNELTKVKYVTEGMRKPAFLSG | |
| | EQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFA | |
| | NRNFMQLIHDDSLTFKEDIQKAQVSGQGDSLHEHIANLAGSPAIKKGIL | |
| | QTVKVVDELVKVMGRHKPENIVIEMARENQTTQKGQKNSRERMKRIE | |
| | EGIKELGSQILKEHPVENTQLQNEKLYLYYLQNGRDMYVDQELDINRLS | |
| | DYDVDHIVPQSFLKDDSIDNKVLTRSDKNRGKSDNVPSEEVVKKMKNY | |
| | WRQLLNAKLITQRKFDNLTKAERGGLSELDKAGFIKRQLVETRQITKHV | |
| | AQILDSRMNTKYDENDKLIREVKVITLKSKLVSDFRKDFQFYKVREINN | |
| | YHHAHDAYLNAVVGTALIKKYPKLESEFVYGDYKVYDVRKMIAKSEQ | |
| | EIGKATAKYFFYSNIMNFFKTEITLANGEIRKRPLIETNGETGEIVWDKG | |
| | RDFATVRKVLSMPQVNIVKKTEVQTGGFSKESILPKRNSDKLIARKKD | |
| | WDPKKYGGFVSPTVAYSVLVVAKVEKGKSKKLKSVKELLGITIMERSS | |
| | FEKNPIDFLEAKGYKEVKKDLIIKLPKYSLFELENGRKRMLASARELQK | |
| | GNELALPSKYVNFLYLASHYEKLKGSPEDNEQKQLFVEQHKHYLDEIIE | |
| | QISEFSKRVILADANLDKVLSAYNKHRDKPIREQAENIIHLFTLTNLGAP | |
| | AAFKYFDTTIDRKQYRSTKEVLDATLIHQSITGLYETRIDLSQLGGD | |
| SpCas9- | DKKYSIGLAIGTNSVGWAVITDEYKVPSKKFKVLGNTDRHSIKKNLIGA | 283 |
| NG | LLFDSGETAEATRLKRTARRRYTRRKNRICYLQEIFSNEMAKVDDSFFH | |
| | RLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFE | |
| | ENPINASGVDAKAILSARLSKSRRLENLIAQLPGEKKNGLFGNLIALSLG | |
| | LTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPE | |
| | KYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILT | |
| | FRIPYYVGPLARGNSRFAWMTRKSEETITPWNFEEVVDKGASAQSFIER | |
| | MTNFDKNLPNEKVLPKHSLLYEYFTVYNELTKVKYVTEGMRKPAFLSG | |
| | EQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFA | |
| | NRNFMQLIHDDSLTFKEDIQKAQVSGQGDSLHEHIANLAGSPAIKKGIL | |
| | QTVKVVDELVKVMGRHKPENIVIEMARENQTTQKGQKNSRERMKRIE | |
| | EGIKELGSQILKEHPVENTQLQNEKLYLYYLQNGRDMYVDQELDINRLS | |
| | DYDVDHIVPQSFLKDDSIDNKVLTRSDKNRGKSDNVPSEEVVKKMKNY | |
| | WRQLLNAKLITQRKFDNLTKAERGGLSELDKAGFIKRQLVETRQITKHV | |
| | AQILDSRMNTKYDENDKLIREVKVITLKSKLVSDFRKDFQFYKVREINN | |
| | YHHAHDAYLNAVVGTALIKKYPKLESEFVYGDYKVYDVRKMIAKSEQ | |
| | EIGKATAKYFFYSNIMNFFKTEITLANGEIRKRPLIETNGETGEIVWDKG | |
| | RDFATVRKVLSMPQVNIVKKTEVQTGGFSKESIRPKRNSDKLIARKKD | |
| | WDPKKYGGFVSPTVAYSVLVVAKVEKGKSKKLKSVKELLGITIMERSS | |
| | FEKNPIDFLEAKGYKEVKKDLIIKLPKYSLFELENGRKRMLASARFLQK | |
| | GNELALPSKYVNFLYLASHYEKLKGSPEDNEQKQLFVEQHKHYLDEIIE | |
| | QISEFSKRVILADANLDKVLSAYNKHRDKPIREQAENIIHLFTLTNLGAP | |
| | RAFKYFDTTIDRKVYRSTKEVLDATLIHQSITGLYETRIDLSQLGGD | |
| SpCas9- | DKKYSIGLTIGTNSVGWAVITDEYKVPSKKFKVLGNTDRHSIKKNLIGA | 284 |
| NRCH | LLFDSGETAEATRLKRTARRRYTRRKNRICYLQEIFSNEMAKVDDSFFH | |
| | RLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFE | |
| | ENPINASGVDAKAILSARLSKSRRLENLIAQLPGEKKNGLFGNLIALSLG | |
| | LTPNFKSNFDLAEDAKLQLSKDTYDDDLDNLLAQIGDQYADLFLAAKN | |
| | LSDAILLSDILRVNTEITKAPLSASMVKRYDEHHQDLTLLKALVRQQLP | |
| | EKYKEIFFDQSKNGYAGYIDGGASQEEFYKFIKPILEKMDGTEELLVKL | |
| | NREDLLRKQRTFDNGIIPHQIHLGELHAILRRQGDFYPFLKDNREKIEKIL | |
| | TFRIPYYVGPLARGNSRFAWMTRKSEETITPWNFEEVVDKGASAQSFIE | |
| | RMTNFDKNLPNEKVLPKHSLLYEYFTVYNELTKVKYVTEGMRKPAFLS | |
| | GEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYA | |
| | HLFDDKVMKQLKRLRYTGWGRLSRKLINGIRDKQSGKTILDFLKSDGF | |
| | ANRNFMQLIHDDSLTFKEDIQKAQVSGQGDSLHEHIANLAGSPAIKKGI | |
| | LQTVKVVDELVKVMGGHKPENIVIEMARENQTTQKGQKNSRERMKRI | |
| | EEGIKELGSQILKEHPVENTQLQNEKLYLYYLQNGRDMYVDQELDINR | |
| | LSDYDVDHIVPQSFLKDDSIDNKVLTRSDKNRGKSDNVPSEEVVKKMK | |
| | NYWRQLLNAKLITQRKFDNLTKAERGGLSELDKAGFIKRQLVETRQITK | |
| | HVAQILDSRMNTKYDENDKLIREVKVITLKSKLVSDFRKDFQFYKVREI | |
| | NNYHHAHDAYLNAVVGTALIKKYPKLESEFVYGDYKVYDVRKMIAKS | |
| | EQEIGKATAKYFFYSNIMNFFKTEITLANGEIRKRPLIETNGETGEIVWD | |
| | KGRDFATVRKVLSMPQVNIVKKTEVQTGGFSKESILPKGNSDKLIARKK | |
| | DWDPKKYGGFNSPTVAYSVLVVAKVEKGKSKKLKSVKELLGITIMERS | |
| | SFEKNPIDFLEAKGYKEVKKDLIIKLPKYSLFELENGRKRMLASAGVLQ | |
| | KGNELALPSKYVNFLYLASHYEKLKGSPEDNEQKQLFVEQHKHYLDEII | |
| | EQISEFSKRVILADANLDKVLSAYNKHRDKPIREQAENIIHLFTLTNLGA | |
| | PAAFKYFDTTINRKQYNTTKEVLDATLIRQSITGLYETRIDLSQLGGD | |
| SpCas9- | DKKYSIGLTIGTNSVGWAVITDEYKVPSKKFKVLGNTDRHSIKKNLIGA | 285 |
| NRTH | LLFDSGETAEATRLKRTARRRYTRRKNRICYLQEIFSNEMAKVDDSFFH | |
| | RLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFE | |
| | ENPINASGVDAKAILSARLSKSRRLENLIAQLPGEKKNGLFGNLIALSLG | |
| | LTPNFKSNFDLAEDAKLQLSKDTYDDDLDNLLAQIGDQYADLFLAAKN | |
| | LSDAILLSDILRVNTEITKAPLSASMVKRYDEHHQDLTLLKALVRQQLP | |
| | EKYKEIFFDQSKNGYAGYIDGGASQEEFYKFIKPILEKMDGTEELLVKL | |
| | NREDLLRKQRTFDNGIIPHQIHLGELHAILRRQGDFYPFLKDNREKIEKIL | |
| | TFRIPYYVGPLARGNSRFAWMTRKSEETITPWNFEEVVDKGASAQSFIE | |
| | RMTNFDKNLPNEKVLPKHSLLYEYFTVYNELTKVKYVTEGMRKPAFLS | |
| | GEQKKAIVDLLFKTNRKVTVKQLKEDYFKKIECFDSVEISGVEDRFNAS | |
| | LGTYHDLLKIIKDKDFLDNEENEDILEDIVLTLTLFEDREMIEERLKTYA | |
| | HLFDDKVMKQLKRLRYTGWGRLSRKLINGIRDKQSGKTILDFLKSDGF | |
| | ANRNFMQLIHDDSLTFKEDIQKAQVSGQGDSLHEHIANLAGSPAIKKGI | |
| | LQTVKVVDELVKVMGGHKPENIVIEMARENQTTQKGQKNSRERMKRI | |
| | EEGIKELGSQILKEHPVENTQLQNEKLYLYYLQNGRDMYVDQELDINR | |
| | LSDYDVDHIVPQSFLKDDSIDNKVLTRSDKNRGKSDNVPSEEVVKKMK | |
| | NYWRQLLNAKLITQRKFDNLTKAERGGLSELDKAGFIKRQLVETRQITK | |
| | HVAQILDSRMNTKYDENDKLIREVKVITLKSKLVSDFRKDFQFYKVREI | |
| | NNYHHAHDAYLNAVVGTALIKKYPKLESEFVYGDYKVYDVRKMIAKS | |
| | EQEIGKATAKYFFYSNIMNFFKTEITLANGEIRKRPLIETNGETGEIVWD | |
| | KGRDFATVRKVLSMPQVNIVKKTEVQTGGFSKESILPKGNSDKLIARKK | |
| | DWDPKKYGGFNSPTVAYSVLVVAKVEKGKSKKLKSVKELLGITIMERS | |
| | SFEKNPIGFLEAKGYKEVKKDLIIKLPKYSLFELENGRKRMLASASVLH | |
| | KGNELALPSKYVNFLYLASHYEKLKGSSEDNKQKQLFVEQHKHYLDEII | |
| | EQISEFSKRVILADANLDKVLSAYNKHRDKPIREQAENIIHLFTLTNLGA | |
| | SAAFKYFDTTIGRKLYTSTKEVLDATLIHQSITGLYETRIDLSQLGGD | |
| dSpCas9 | DKKYSIGLAIGTNSVGWAVITDEYKVPSKKFKVLGNTDRHSIKKNLIGA | 286 |
| | LLFDSGETAEATRLKRTARRRYTRRKNRICYLQEIFSNEMAKVDDSFFH | |
| | RLEESFLVEEDKKHERHPIFGNIVDEVAYHEKYPTIYHLRKKLVDSTDK | |
| | ADLRLIYLALAHMIKFRGHFLIEGDLNPDNSDVDKLFIQLVQTYNQLFE | |
| | ENPINASGVDAKAILSARLSKSRRLENLIAQLPGEKKNGLFGNLIALSLG | |
| | LTPNFKSNFDLAEDAKLQLSKDTYDDDLDNLLAQIGDQYADLFLAAKN | |
| | LSDAILLSDILRVNTEITKAPLSASMIKRYDEHHQDLTLLKALVRQQLPE | |
| | KYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILT | |
| | FRIPYYVGPLARGNSRFAWMTRKSEETITPWNFEEVVDKGASAQSFIER | |
| | MTNFDKNLPNEKVLPKHSLLYEYFTVYNELTKVKYVTEGMRKPAFLSG | |
| | EQKKAIVDLLFKTNRKVTVKQLKEDYFKKIECFDSVEISGVEDRFNASL | |
| | GTYHDLLKIIKDKDFLDNEENEDILEDIVLTLTLFEDREMIEERLKTYAH | |
| | LFDDKVMKQLKRRRYTGWGRLSRKLINGIRDKQSGKTILDFLKSDGFA | |
| | NRNFMQLIHDDSLTFKEDIQKAQVSGQGDSLHEHIANLAGSPAIKKGIL | |
| | QTVKVVDELVKVMGRHKPENIVIEMARENQTTQKGQKNSRERMKRIE | |
| | EGIKELGSQILKEHPVENTQLQNEKLYLYYLQNGRDMYVDQELDINRLS | |
| | DYDVDAIVPQSFLKDDSIDNKVLTRSDKNRGKSDNVPSEEVVKKMKNY | |
| | WRQLLNAKLITQRKFDNLTKAERGGLSELDKAGFIKRQLVETRQITKHV | |
| | AQILDSRMNTKYDENDKLIREVKVITLKSKLVSDFRKDFQFYKVREINN | |
| | YHHAHDAYLNAVVGTALIKKYPKLESEFVYGDYKVYDVRKMIAKSEQ | |
| | EIGKATAKYFFYSNIMNFFKTEITLANGEIRKRPLIETNGETGEIVWDKG | |
| | RDFATVRKVLSMPQVNIVKKTEVQTGGFSKESILPKRNSDKLIARKKD | |
| | WDPKKYGGFDSPTVAYSVLVVAKVEKGKSKKLKSVKELLGITIMERSS | |
| | FEKNPIDFLEAKGYKEVKKDLIIKLPKYSLFELENGRKRMLASAGELQK | |
| | GNELALPSKYVNFLYLASHYEKLKGSPEDNEQKQLFVEQHKHYLDEIIE | |
| | QISEFSKRVILADANLDKVLSAYNKHRDKPIREQAENIIHLFTLTNLGAP | |
| | AAFKYFDTTIDRKRYTSTKEVLDATLIHQSITGLYETRIDLSQLGGD | |
| SaCas9 | GKRNYILGLAIGITSVGYGIIDYETRDVIDAGVRLFKEANVENNEGRRSK | 287 |
| | RGARRLKRRRRHRIQRVKKLLFDYNLLTDHSELSGINPYEARVKGLSQK | |
| | LSEEEFSAALLHLAKRRGVHNVNEVEEDTGNELSTKEQISRNSKALEEK | |
| | YVAELQLERLKKDGEVRGSINRFKTSDYVKEAKQLLKVQKAYHQLDQ | |
| | SFIDTYIDLLETRRTYYEGPGEGSPFGWKDIKEWYEMLMGHCTYFPEEL | |
| | RSVKYAYNADLYNALNDLNNLVITRDENEKLEYYEKFQIIENVFKQKK | |
| | KPTLKQIAKEILVNEEDIKGYRVTSTGKPEFTNLKVYHDIKDITARKEIIE | |
| | NAELLDQIAKILTIYQSSEDIQEELTNLNSELTQEEIEQISNLKGYTGTHN | |
| | LSLKAINLILDELWHTNDNQIAIFNRLKLVPKKVDLSQQKEIPTTLVDDFI | |
| | LSPVVKRSFIQSIKVINAIIKKYGLPNDIIIELAREKNSKDAQKMINEMQK | |
| | RNRQTNERIEEIIRTTGKENAKYLIEKIKLHDMQEGKCLYSLEAIPLEDLL | |
| | NNPFNYEVDHIIPRSVSFDNSFNNKVLVKQEENSKKGNRTPFQYLSSSDS | |
| | KISYETFKKHILNLAKGKGRISKTKKEYLLEERDINRFSVQKDFINRNLV | |
| | DTRYATRGLMNLLRSYFRVNNLDVKVKSINGGFTSFLRRKWKFKKERN | |
| | KGYKHHAEDALIIANADFIFKEWKKLDKAKKVMENQMFEEKQAESMP | |
| | EIETEQEYKEIFITPHQIKHIKDFKDYKYSHRVDKKPNRELINDTLYSTRK | |
| | DDKGNTLIVNNLNGLYDKDNDKLKKLINKSPEKLLMYHHDPQTYQKL | |
| | KLIMEQYGDEKNPLYKYYEETGNYLTKYSKKDNGPVIKKIKYYGNKLN | |
| | AHLDITDDYPNSRNKVVKLSLKPYRFDVYLDNGVYKFVTVKNLDVIKK | |
| | ENYYEVNSKCYEEAKKLKKISNQAEFIASFYNNDLIKINGELYRVIGVN | |
| | NDLLNRIEVNMIDITYREYLENMNDKRPPRIIKTIASKTQSIKKYSTDILG | |
| | NLYEVKSKKHPQIIKKG | |
| SaKKH | GKRNYILGLAIGITSVGYGIIDYETRDVIDAGVRLFKEANVENNEGRRSK | 288 |
| Cas9 | RGARRLKRRRRHRIQRVKKLLFDYNLLTDHSELSGINPYEARVKGLSQK | |
| | LSEEEFSAALLHLAKRRGVHNVNEVEEDTGNELSTKEQISRNSKALEEK | |
| | YVAELQLERLKKDGEVRGSINRFKTSDYVKEAKQLLKVQKAYHQLDQ | |
| | SFIDTYIDLLETRRTYYEGPGEGSPFGWKDIKEWYEMLMGHCTYFPEEL | |
| | RSVKYAYNADLYNALNDLNNLVITRDENEKLEYYEKFQIIENVFKQKK | |
| | KPTLKQIAKEILVNEEDIKGYRVTSTGKPEFTNLKVYHDIKDITARKEIIE | |
| | NAELLDQIAKILTIYQSSEDIQEELTNLNSELTQEEIEQISNLKGYTGTHN | |
| | LSLKAINLILDELWHTNDNQIAIFNRLKLVPKKVDLSQQKEIPTTLVDDFI | |
| | LSPVVKRSFIQSIKVINAIIKKYGLPNDIIIELAREKNSKDAQKMINEMQK | |
| | RNRQTNERIEEIIRTTGKENAKYLIEKIKLHDMQEGKCLYSLEAIPLEDLL | |
| | NNPFNYEVDHIIPRSVSFDNSFNNKVLVKQEENSKKGNRTPFQYLSSSDS | |
| | KISYETFKKHILNLAKGKGRISKTKKEYLLEERDINRFSVQKDFINRNLV | |
| | DTRYATRGLMNLLRSYFRVNNLDVKVKSINGGFTSFLRRKWKFKKERN | |
| | KGYKHHAEDALIIANADFIFKEWKKLDKAKKVMENQMFEEKQAESMP | |
| | EIETEQEYKEIFITPHQIKHIKDFKDYKYSHRVDKKPNRKLINDTLYSTRK | |
| | DDKGNTLIVNNLNGLYDKDNDKLKKLINKSPEKLLMYHHDPQTYQKL | |
| | KLIMEQYGDEKNPLYKYYEETGNYLTKYSKKDNGPVIKKIKYYGNKLN | |
| | AHLDITDDYPNSRNKVVKLSLKPYRFDVYLDNGVYKFVTVKNLDVIKK | |
| | ENYYEVNSKCYEEAKKLKKISNQAEFIASFYKNDLIKINGELYRVIGVN | |
| | NDLLNRIEVNMIDITYREYLENMNDKRPPHIIKTIASKTQSIKKYSTDILG | |
| | NLYEVKSKKHPQIIKKG | |
| LbCpf1 | SKLEKFTNCYSLSKTLRFKAIPVGKTQENIDNKRLLVEDEKRAEDYKGV | 289 |
| | KKLLDRYYLSFINDVLHSIKLKNLNNYISLFRKKTRTEKENKELENLEIN | |
| | LRKEIAKAFKGNEGYKSLFKKDIIETILPEFLDDKDEIALVNSFNGFTTAF | |
| | TGFFDNRENMFSEEAKSTSIAFRCINENLTRYISNMDIFEKVDAIFDKHE | |
| | VQEIKEKILNSDYDVEDFFEGEFFNFVLTQEGIDVYNAIIGGFVTESGEKI | |
| | KGLNEYINLYNQKTKQKLPKFKPLYKQVLSDRESLSFYGEGYTSDEEVL | |
| | EVFRNTLNKNSEIFSSIKKLEKLFKNFDEYSSAGIFVKNGPAISTISKDIFG | |
| | EWNVIRDKWNAEYDDIHLKKKAVVTEKYEDDRRKSFKKIGSFSLEQLQ | |
| | EYADADLSVVEKLKEIIIQKVDEIYKVYGSSEKLFDADFVLEKSLKKND | |
| | AVVAIMKDLLDSVKSFENYIKAFFGEGKETNRDESFYGDFVLAYDILLK | |
| | VDHIYDAIRNYVTQKPYSKDKFKLYFQNPQFMGGWDKDKETDYRATIL | |
| | RYGSKYYLAIMDKKYAKCLQKIDKDDVNGNYEKINYKLLPGPNKMLP | |
| | KVFFSKKWMAYYNPSEDIQKIYKNGTFKKGDMFNLNDCHKLIDFFKDS | |
| | ISRYPKWSNAYDFNFSETEKYKDIAGFYREVEEQGYKVSFESASKKEVD | |
| | KLVEEGKLYMFQIYNKDFSDKSHGTPNLHTMYFKLLFDENNHGQIRLS | |
| | GGAELFMRRASLKKEELVVHPANSPIANKNPDNPKKTTTLSYDVYKDK | |
| | RFSEDQYELHIPIAINKCPKNIFKINTEVRVLLKHDDNPYVIGIARGERNL | |
| | LYIVVVDGKGNIVEQYSLNEIINNFNGIRIKTDYHSLLDKKEKERFEARQ | |
| | NWTSIENIKELKAGYISQVVHKICELVEKYDAVIALEDLNSGFKNSRVK | |
| | VEKQVYQKFEKMLIDKLNYMVDKKSNPCATGGALKGYQITNKFESFKS | |
| | MSTQNGFIFYIPAWLTSKIDPSTGFVNLLKTKYTSIADSKKFISSFDRIMY | |
| | VPEEDLFEFALDYKNFSRTDADYIKKWKLYSYGNRIRIFRNPKKNNVFD | |
| | WEEVCLTSAYKELFNKYGINYQQGDIRALLCEQSDKAFYSSFMALMSL | |
| | MLQMRNSITGRTDVDFLISPVKNSDGIFYDSRNYEAQENAILPKNADAN | |
| | GAYNIARKVLWAIGQFKKAEDEKLDKVKIAISNKEWLEYAQTSVK | |
| enAsCpf1 | TQFEGFTNLYQVSKTLRFELIPQGKTLKHIQEQGFIEEDKARNDHYKEL | 290 |
| | KPIIDRIYKTYADQCLQLVQLDWENLSAAIDSYRKEKTEETRNALIEEQA | |
| | TYRNAIHDYFIGRTDNLTDAINKRHAEIYKGLFKAELFNGKVLKQLGTV | |
| | TTTEHENALLRSFDKFTTYFSGFYRNRKNVFSAEDISTAIPHRIVQDNFP | |
| | KFKENCHIFTRLITAVPSLREHFENVKKAIGIFVSTSIEEVFSFPFYNQLLT | |
| | QTQIDLYNQLLGGISREAGTEKIKGLNEVLNLAIQKNDETAHIIASLPHR | |
| | FIPLFKQILSDRNTLSFILEEFKSDEEVIQSFCKYKTLLRNENVLETAEALF | |
| | NELNSIDLTHIFISHKKLETISSALCDHWDTLRNALYERRISELTGKITKS | |
| | AKEKVQRSLKHEDINLQEIISAAGKELSEAFKQKTSEILSHAHAALDQPL | |
| | PTTLKKQEEKEILKSQLDSLLGLYHLLDWFAVDESNEVDPEFSARLTGI | |
| | KLEMEPSLSFYNKARNYATKKPYSVEKFKLNFQMPTLARGWDVNREK | |
| | NNGAILFVKNGLYYLGIMPKQKGRYKALSFEPTEKTSEGFDKMYYDYF | |
| | PDAAKMIPKCSTQLKAVTAHFQTHTTPILLSNNFIEPLEITKEIYDLNNPE | |
| | RPSSQYKDLGEYYAELNPLLYHISFQRIAEKEIMDAVETGKLYLFQIYNK | |
| | KEPKKFQTAYAKKTGDQKGYREALCKWIDFTRDFLSKYTKTTSIDLSSL | |
| | DFAKGHHGKPNLHTLYWTGLFSPENLAKTSIKLNGQAELFYRPKSRMK | |
| | RMAHRLGEKMLNKKLKDQKTPIPDTLYQELYDYVNHRLSHDLSDEAR | |
| | ALLPNVITKEVSHEIIKDRRFTSDKFFFHVPITLNYQAANSPSKFNQRVN | |
| | AYLKEHPETPIIGIARGERNLIYITVIDSTGKILEQRSLNTIQQFDYQKKLD | |
| | NREKERVAARQAWSVVGTIKDLKQGYLSQVIHEIVDLMIHYQAVVVLE | |
| | NLNFGFKSKRTGIAEKAVYQQFEKMLIDKLNCLVLKDYPAEKVGGVLN | |
| | PYQLTDQFTSFAKMGTQSGFLFYVPAPYTSKIDPLTGFVDPFVWKTIKN | |
| | HESRKHFLEGFDFLHYDVKTGDFILHFKMNRNLSFQRGLPGFMPAWDI | |
| | VFEKNETQFDAKGTPFIAGKRIVPVIENHRFTGRYRDLYPANELIALLEE | |
| | KGIVFRDGSNILPKLLENDDSHAIDTMVALIRSVLQMRNSNAATGEDYI | |
| | NSPVRDLNGVCFDSRFQNPEWPMDADANGAYHIALKGQLLLNHLKES | |
| | KDLKLQNGISNQDWLAYIQELRN | |

| TadA8r- | | SEQ |
|---|---|---|
| effector | | ID |
| fusions | Sequence | NO |
| N terminal | MKRTADGSEFESPKKKRKV | 313 |
| BP_NLS | | |
| TadA8r | SEVEFSHEYWMRHALTLAKRARDEREVPVGAVLVLNNRVIGEGWN | 308 |
| | KAIGLHDPTAHAEIMALRQGGLVMQNYRLYDATLYSTLEPCVMCA | |
| | GAMIHSRIGRVVFGVRGARHGAVGSLMNVLHYPGIKHRVEITEGILA | |
| | DECAALLCRFFRMPRRVFKAQKKAQSSTD | |
| 32 amino | SGGSSGGSSGSETPGTSESATPESSGGSSGGS | 314 |
| acid linker | | |
| nSpCas9 | DKKYSIGLAIGTNSVGWAVITDEYKVPSKKFKVLGNTDRHSIKKNLI | 281 |
| | GALLFDSGETAEATRLKRTARRRYTRRKNRICYLQEIFSNEMAKVDD | |
| | SFFHRLEESFLVEEDKKHERHPIFGNIVDEVAYHEKYPTIYHLRKKLV | |
| | DSTDKADLRLIYLALAHMIKFRGHFLIEGDLNPDNSDVDKLFIQLVQ | |
| | TYNQLFEENPINASGVDAKAILSARLSKSRRLENLIAQLPGEKKNGLF | |
| | GNLIALSLGLTPNFKSNFDLAEDAKLQLSKDTYDDDLDNLLAQIGDQ | |
| | YADLFLAAKNLSDAILLSDILRVNTEITKAPLSASMIKRYDEHHQDLT | |
| | LLKALVRQQLPEKYKEIFFDQSKNGYAGYIDGGASQEEFYKFIKPILE | |
| | KMDGTEELLVKLNREDLLRKQRTFDNGSIPHQIHLGELHAILRRQED | |
| | FYPFLKDNREKIEKILTFRIPYYVGPLARGNSRFAWMTRKSEETITPW | |
| | NFEEVVDKGASAQSFIERMTNFDKNLPNEKVLPKHSLLYEYFTVYNE | |
| | LTKVKYVTEGMRKPAFLSGEQKKAIVDLLFKTNRKVTVKQLKEDYF | |
| | KKIECFDSVEISGVEDRFNASLGTYHDLLKIIKDKDFLDNEENEDILED | |
| | IVLTLTLFEDREMIEERLKTYAHLFDDKVMKQLKRRRYTGWGRLSR | |
| | KLINGIRDKQSGKTILDFLKSDGFANRNFMQLIHDDSLTFKEDIQKAQ | |
| | VSGQGDSLHEHIANLAGSPAIKKGILQTVKVVDELVKVMGRHKPENI | |
| | VIEMARENQTTQKGQKNSRERMKRIEEGIKELGSQILKEHPVENTQL | |
| | QNEKLYLYYLQNGRDMYVDQELDINRLSDYDVDHIVPQSFLKDDSI | |
| | DNKVLTRSDKNRGKSDNVPSEEVVKKMKNYWRQLLNAKLITQRKF | |
| | DNLTKAERGGLSELDKAGFIKRQLVETRQITKHVAQILDSRMNTKYD | |
| | ENDKLIREVKVITLKSKLVSDFRKDFQFYKVREINNYHHAHDAYLNA | |
| | VVGTALIKKYPKLESEFVYGDYKVYDVRKMIAKSEQEIGKATAKYF | |
| | FYSNIMNFFKTEITLANGEIRKRPLIETNGETGEIVWDKGRDFATVRK | |
| | VLSMPQVNIVKKTEVQTGGFSKESILPKRNSDKLIARKKDWDPKKY | |
| | GGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPA | |
| | AFKYFDTTIDRKRYTSTKEVLDATLIHQSITGLYETRIDLSQLGGD | |
| 4 amino | SGGS | 315 |
| acid linker | | |
| C teminal | KRTADGSEFEPKKKRKV | 316 |
| BP_NLS | | |
| NLS- | MKRTADGSEFESPKKKRKVSEVEFSHEYWMRHALTLAKRARDERE | 317 |
| TadA8r-32 | VPVGAVLVLNNRVIGEGWNKAIGLHDPTAHAEIMALRQGGLVMQN | |
| amino acid | YRLYDATLYSTLEPCVMCAGAMIHSRIGRVVFGVRGARHGAVGSL | |
| linker- | MNVLHYPGIKHRVEITEGILADECAALLCRFFRMPRRVFKAQKKAQS | |
| nSpCas9- | STDSGGSSGGSSGSETPGTSESATPESSGGSSGGSDKKYSIGLAIGTNS | |
| linker-NLS | VGWAVITDEYKVPSKKFKVLGNTDRHSIKKNLIGALLFDSGETAEAT | |
| | RLKRTARRRYTRRKNRICYLQEIFSNEMAKVDDSFFHRLEESFLVEE | |
| | DKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINAS | |
| | GVDAKAILSARLSKSRRLENLIAQLPGEKKNGLFGNLIALSLGLTPNF | |
| | KSNFDLAEDAKLQLSKDTYDDDLDNLLAQIGDQYADLFLAAKNLSD | |
| | AILLSDILRVNTEITKAPLSASMIKRYDEHHQDLTLLKALVRQQLPEK | |
| | YKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKI | |
| | LTFRIPYYVGPLARGNSRFAWMTRKSEETITPWNFEEVVDKGASAQS | |
| | FIERMTNFDKNLPNEKVLPKHSLLYEYFTVYNELTKVKYVTEGMRK | |
| | PAFLSGEQKKAIVDLLFKTNRKVTVKQLKEDYFKKIECFDSVEISGVE | |
| | DRFNASLGTYHDLLKIIKDKDFLDNEENEDILEDIVLTLTLFEDREMIE | |
| | ERLKTYAHLFDDKVMKQLKRRRYTGWGRLSRKLINGIRDKQSGKTI | |
| | LDFLKSDGFANRNFMQLIHDDSLTFKEDIQKAQVSGQGDSLHEHIAN | |
| | LAGSPAIKKGILQTVKVVDELVKVMGRHKPENIVIEMARENQTTQK | |
| | GQKNSRERMKRIEEGIKELGSQILKEHPVENTQLQNEKLYLYYLQNG | |
| | RDMYVDQELDINRLSDYDVDHIVPQSFLKDDSIDNKVLTRSDKNRG | |
| | KSDNVPSEEVVKKMKNYWRQLLNAKLITQRKFDNLTKAERGGLSEL | |
| | DKAGFIKRQLVETRQITKHVAQILDSRMNTKYDENDKLIREVKVITL | |
| | KSKLVSDFRKDFQFYKVREINNYHHAHDAYLNAVVGTALIKKYPKL | |
| | ESEFVYGDYKVYDVRKMIAKSEQEIGKATAKYFFYSNIMNFFKTEIT | |
| | LANGEIRKRPLIETNGETGEIVWDKGRDFATVRKVLSMPQVNIVKKT | |
| | EVQTGGFSKESILPKRNSDKLIARKKDWDPKKYGGFDSPTVAYSVLV | |
| | VAKVEKGKSKKLKSVKELLGITIMERSSFEKNPIDFLEAKGYKEVKK | |
| | DLIIKLPKYSLFELENGRKRMLASAGELQKGNELALPSKYVNFLYLA | |
| | SHYEKLKGSPEDNEQKQLFVEQHKHYLDEIIEQISEFSKRVILADANL | |
| | DKVLSAYNKHRDKPIREQAENIIHLFTLTNLGAPAAFKYFDTTIDRKR | |
| | YTSTKEVLDATLIHQSITGLYETRIDLSQLGGDSGGSKRTADGSEFEP | |
| | KKKRKV | |

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Directed Evolution of an Adenine Base Editor with Improved Activity and Altered Context Preference

Approximately 60% of known disease-associated genetic variations in the human genome are point mutations, close to half of which are G:C to A:T transitions (1, 2). Adenine base editors (ABEs), wherein a deoxyadenosine deaminase is covalently linked to a catalytically impaired CRISPR protein via a flexible linker, can correct G:C to A:T mutations site-specifically in the genome without introducing excessive double-stranded DNA (dsDNA) breaks (3, 4). The deoxyadenosine deaminases in ABEs are variants of the Escherichia coli tRNA-specific adenosine deaminase (TadA) (5) evolved to function on single-stranded DNA (ssDNA). While ABE activity was initially demonstrated with Streptococcus pyogenes Cas9 (SpCas9), more CRISPR proteins have since been demonstrated compatible with TadA variants for adenine base editing (6-8). Correction of disease relevant mutations has been achieved with ABEs in a variety of cell models and organisms (9-17), including non-human primates (18-20).

Seven rounds of directed evolution in E. coli yielded TadA7.10 (3), the TadA variant that comprises the state-of-the-art ABE-ABE7.10—wherein a SpCas9 nickase (nCas9) is employed for target DNA engagement. ABE7.10 edits A into G in a window spanning protospacer positions 4-7 through an inosine (I) intermediate. TadA7.10 is most efficient in deaminating A in a “YA” motif (Y: pyrimidine; T and C) (3), a context preference inherited from WT TadA that deaminates adenosine in the anti-codon loop (U)ACG of Arg tRNA (5). This context bias is most evident when the target A is outside the strong editing window (21). TadA7.10, as is evolved in a SpCas9-guided manner, is less compatible with other CRISPR systems. More active TadA variants, TadA8 (22) and TadA8e (7), were obtained by pushing TadA7.10 through additional rounds of directed evolution with increased selection stringencies. ABE8e is 590-fold faster than ABE7.10 under single turnover conditions (7). With substantially improved deamination activity, ABE8 and ABE8e demonstrated universally higher activity and a broadened editing window (4-8) in human cells (7, 22). These high-activity ABEs can be particularly useful for editing disease-causing mutations in primary cells and in vivo where superior activity is required to compensate deficiency in delivery.

TadA8 and TadA8e, both of which are derivatives of TadA7.10, have inherited the weak “YA” context preference (7, 22, 23). Adenine following a purine (RA, R=A or G) remains a challenging substrate, especially when the target A is outside the most optimal editing window (5-7). We set out to overcome this context dependence of TadA by directed evolution. We started with wildtype (WT) E. coli TadA and designed an evolution campaign to force TadA variants to deaminate A in a “GA” context with fast kinetics. Three rounds of de novo directed evolution followed by DNA shuffling led to TadA8r, a TadA variant that outperforms TadA8 and TadA8e in a “RA” motif without losing activity on “YA”. The de novo harvested mutations in TadA8r (36%, 8 out of 22) are critical for this altered context preference. TadA8r has a shifted editing window when fused to SpCas9 and enables more robust editing at protospacer adjacent motif (PAM) distal positions. Similar to TadA8e, TadA8r is broadly compatible with CRISPR effector proteins including SpCas9 with altered and broadened PAM specificities (24, 25, 26), Staphylococcus aureus Cas9 (SaCas9) (27, 28), Lachnospiraceae bacterium Cas12a (LbCas12a) (29), and Acidaminococcus Cas12a (AsCas12a) (29, 30). ABE8r shows lower off-target DNA and RNA editing compared to ABE8e. The off-target effects of ABE8r can be further reduced by introducing a V106W (31) substitution and mRNA delivery. ABE8r outperforms ABE7.10, ABE8.20, and ABE8e in editing several disease-relevant mutations. The orthogonally evolved ABE8r therefore complements and expands the current ABE family with superior activity and altered context preferences.

### A. Results

### 1. De Novo Directed Evolution of TadA

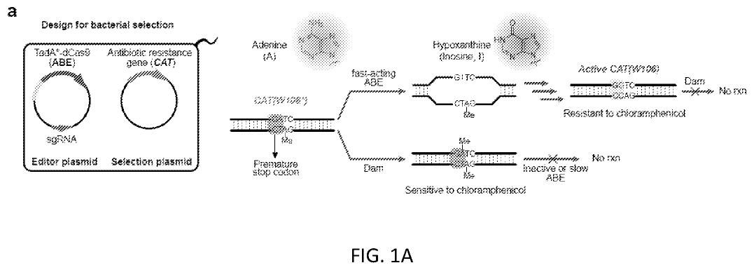

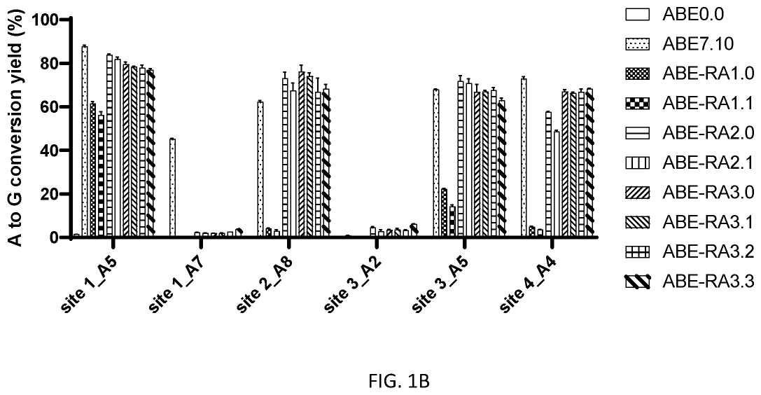

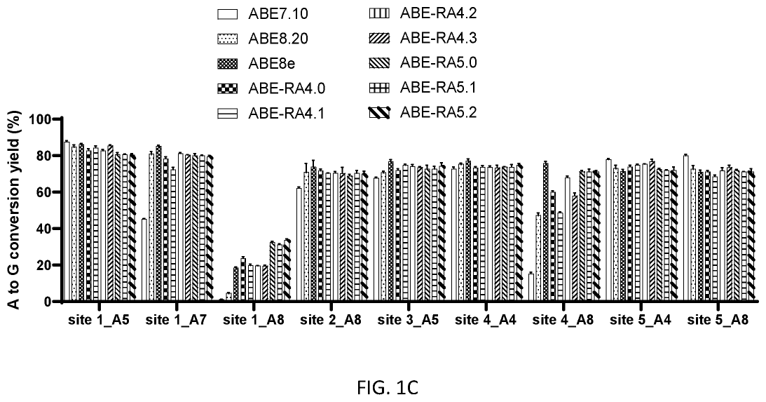

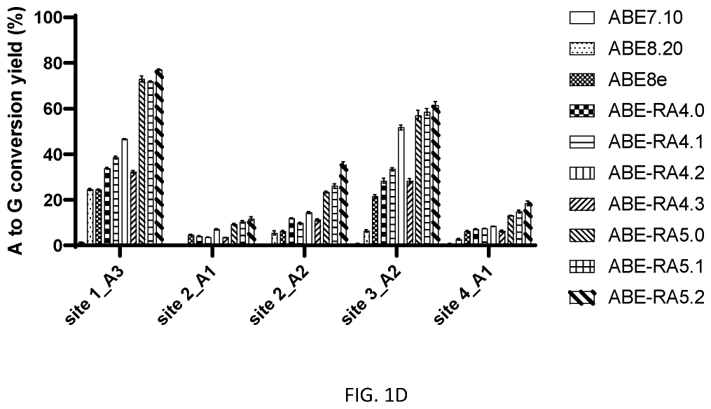

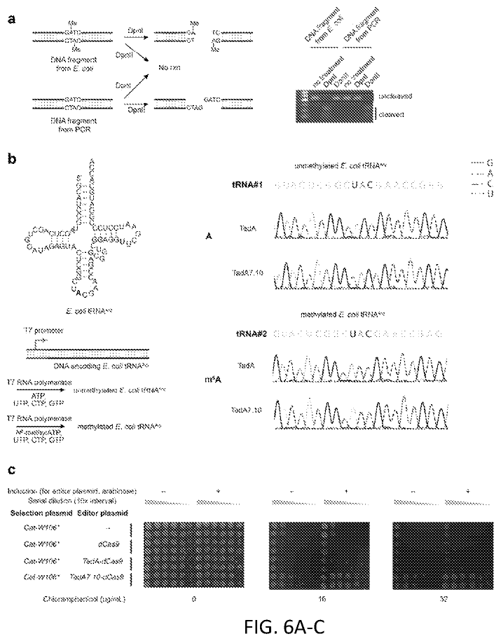

We set out to identify TadA variants that function robustly on deoxyadenosine in “RA” sequences. Our directed evolution scheme is derived from the bacterial selection strategy that yielded TadA7.10 (3) and TadA8.20 (22). Mutation-bearing TadA proteins are recruited to one or more A:T base pairs that inactivate an antibiotic resistance gene (FIG. 1a). Active TadA variants are isolated by collecting bacteria that confer resistance to antibiotic challenges. To route the evolution trajectory of TadA, we placed the target A in a “GATC” context. In E. coli all As in “GATC” sequences are methylated at the N⁶ position by the DNA adenine methyltransferase (Dam) with rare exceptions (FIG. 6a) (32). Hemimethylated “GATC” sites are generated transiently during DNA replication and only persist for a short time (33). We posit it is unlikely for TadA to acquire activity on N⁶-methyldeoxyadenosine through evolution because deamination of N⁶-methyldeoxyadenosine requires hydrolytic removal of methylamine instead of ammonia and wildtype TadA as well as TadA7.10 fully rejects N⁶-methyladenosine in a tRNA substrate (FIG. 6b). Collectively, this design will not only force TadA to accept RA, but also impose strong selection pressure for ultra-fast deamination as TadA needs to compete with Dam for the substrate.

We targeted an A that inactivates the chloramphenicol acetyl transferase gene via a premature stop codon (Cam^{R}-W106*) in first-round selection. Successful deamination introduces an A:T to G:C mutation to Cam^{R}-W106* and fully restores protein activity. While E. coli carrying nuclease deficient Cas9 (dCas9) and TadA-dCas9 succumbed to chloramphenicol challenges, E. coli bearing TadA7.10-dCas9 showed strong survival under the same conditions (FIG. 6c), validating our selection strategy.

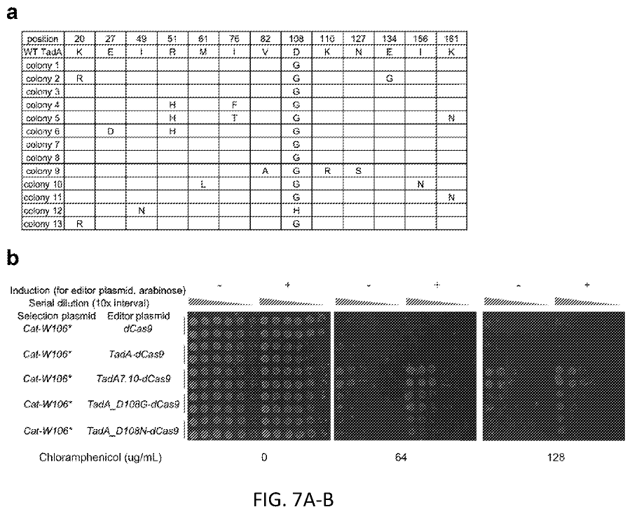

We constructed a TadA library via error prone PCR and cloned this library into the editor plasmid. Bacteria that conferred chloramphenicol resistance were collected. Hits were further validated by subcloning. All survival clones but one contain a D108G mutation (FIG. 7a). D108N was the initial mutation isolated during the evolution of TadA7.10 and was believed to be a critical mutation that enables TadA to function on ssDNA (3, 34). We therefore compared the performance of TadA-D108G and TadA-D108N in our bacterial selection assay. E. coli expressing TadA-D108G-dCas9 survived 64 and 128 μg/mL chloramphenicol with titers 10-fold higher than those expressing TadA-D108N-dCas9 (FIG. 7b), confirming the D108G variant arose in our selection because of efficient deamination of A in “GATC”, rather than codon bias introduced during library construction (35). Three additional consensus mutations emerged in our first-round selection, including K20R, R51H, and K161N. We moved forward with TadA-RA1.0 (D108G) and TadA-RA1.1 (D108G and K161N, Table 1).

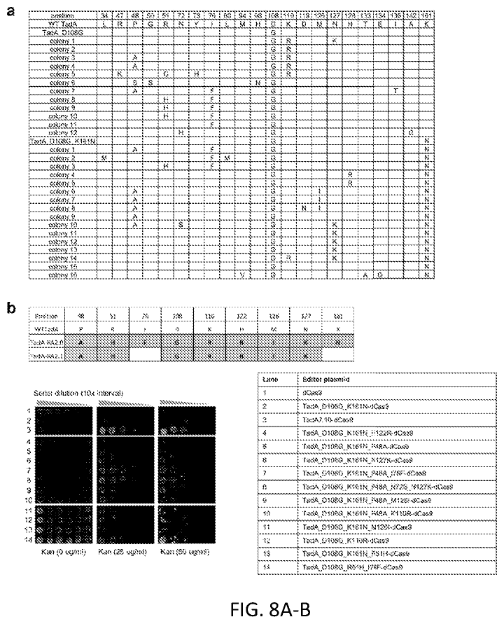

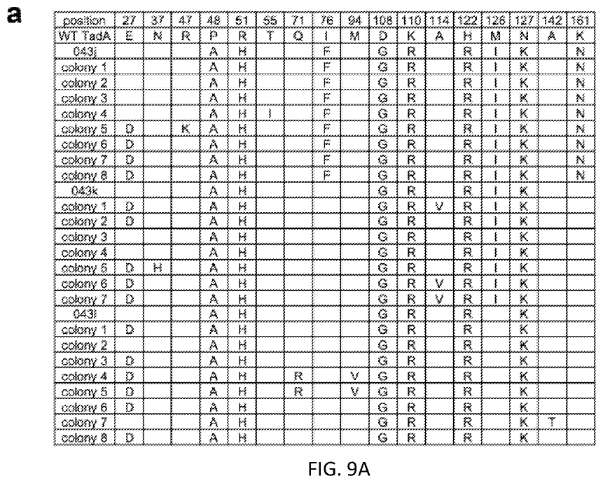

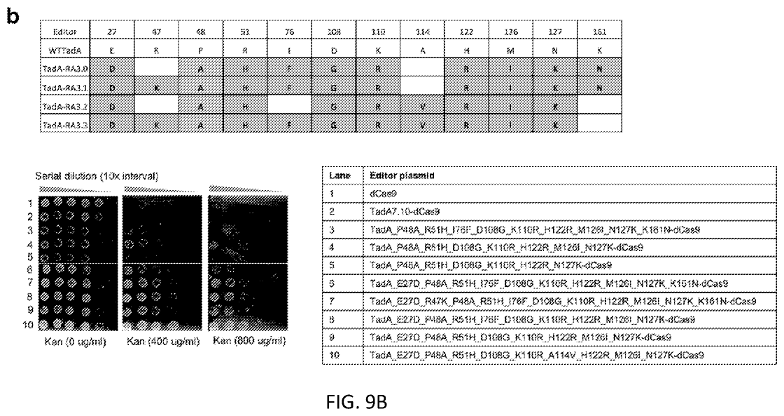

TadA-RA1.0 and TadA-RA1.1 were diversified and subject to second-round selection. To accelerate the accumulation of beneficial mutations, we increased the selection stringency by targeting two premature stop codons surpassing “GATC” in a kanamycin resistance gene (aminoglycoside-3-phosphotransferase, Kan^{R}-W15*W24*). Seven consensus mutations (P48A, R51H, I76F, K110R, H122R, M126I and N127K) emerged in different survival clones, all of which were confirmed beneficial using the bacterial selection assay (Table 1, FIGS. 8a and 8b). These beneficial mutations were incorporated into ABE-RA1.0 and ABE-RA1.1 to form ABE-RA2.0 and ABE-RA2.1. We moved forward with TadA-RA2.0 and TadA-RA2.1 as starting template for error prone PCR. A third round of de novo directed evolution was carried out using Kan^{R}-W15*W24* with higher antibiotic concentration, during which three additional beneficial mutations were isolated: E27D, R47K, A114V (FIGS. 9a and 9b). Note that all mutants evaluated at this stage are substantially more active than TadA7.10 in the bacterial selection assay, resulting in at least two orders of magnitude more survival clones (FIG. 9b). Importantly, mutations we harvested in three rounds of de novo directed evolution do not overlap with mutations hosted by TadA7.10 and TadA7.10-derived TadA8s except P48A. We posit that the RA-only substrate spectrum and the initial acquisition of D108G may have driven our evolution onto an evolution trajectory different from that of TadA7.10.

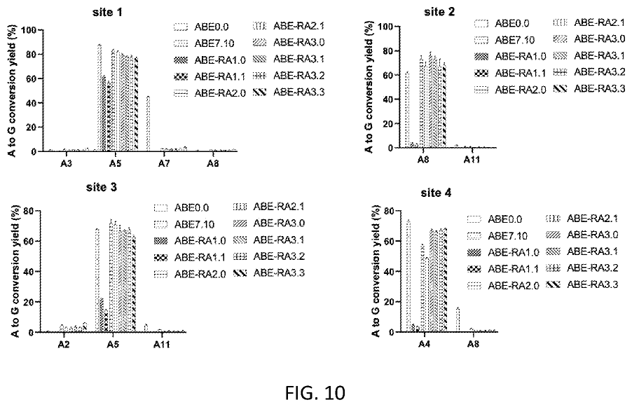

With 12 beneficial mutations identified through de novo evolution, we next characterized representative combinations in mammalian cells. The WT TadA monomer in adenine base editors was found dispensable for editing activity (36), we therefore evaluated TadA variants as TadA*-Cas9 D10A nickase (nCas9) fusion proteins (ABE-RA). Plasmids encoding ABE-RA 1.0, 1.1, 2.0, 2.1, 3.0, 3.1, 3.2, 3.3 and ABE7.10 were delivered into human embryonic kidney (HEK) 293T cells via lipid-mediated transfection with sgRNA plasmids targeting 4 sites on human chromosomes 3, 5, and 6 (FIG. 1b and FIG. 10). Activity accumulation is evident as mutations in more advanced evolution rounds are included. When targeting A in a “GA” motif (A₈ in site 2, A₅ in site 3 and A4 in site 4, in which subscript numbers denote positions in the protospacer), ABE-RA2.0-3.3 delivered 66.8-76.0%, 62.8-71.8% and 48.6-68.1%, a level comparable with ABE7.10 (62.2±0.7%, 67.8±0.3% and 72.8±1.0%, mean±standard deviation, respectively). Specifically, ABE-RA2.0-3.3 outperformed ABE7.10 globally at site 2 (67.3-76.0% versus 62.2%), indicating TadA was rapidly evolved with our de novo scheme. ABE-RA2.0-3.3 generated robust editing at CA₅ in site 1 and TA₄ in site 4 (76.8-83.8% and 62.8-71.8% compared to 87.6±0.7% and 72.8±1.0% by ABE7.10), but showed markedly reduced activity when targeting YA closer to PAM (CA₇ in site 1 and CA₈ in site 4, 1.9-3.7% and 1.0-1.9%, comparing with 45.2% and 15.2% (FIG. 10). Taken together, these results confirm that TadA variants isolated by our de novo directed evolution deaminate deoxyadenosine with an altered context preference.

2. DNA Shuffling with Known Base-Editing Enabling TadA Mutations

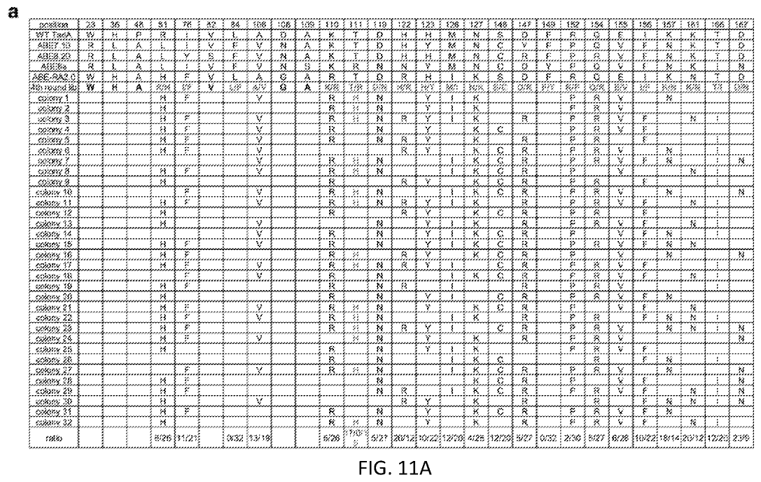

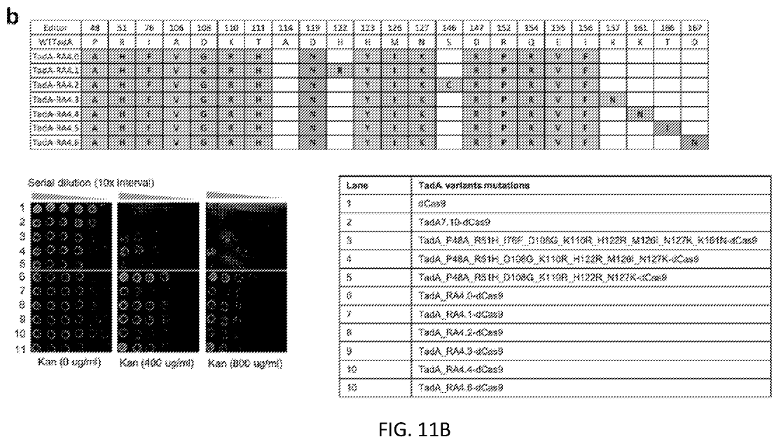

To accelerate the evolution and to recover TadA's activity on “YA” sequences, we next shuffled our de novo acquired mutations with those in TadA7.10, TadA8.20, and TadA8e. We fixed D108G and sorted through more than 30 mutations in two rounds of DNA shuffling. At each of the mutation site, we dosed 1:1 ratio of wildtype amino acid with evolved mutations in the library. The first round of DNA shuffling, or the fourth round of evolution, was carried out using the selection plasmid encoding Kan^{R}-W15*W24*. R51H, K110R, D119N, H123Y, N127K, D147R, R152P, Q154R, E155V, and I156F were strongly enriched (FIG. 11), indicating that these mutations are critical for TadA to function on ssDNA. In contrast, L84F and F149Y were completely absent in survival clones (FIG. 11), suggesting these two mutations are incompatible with the local evolutionary optimum where the current TadA sequence lands. Other mutations are mostly neutral, i.e., either enriched or depleted from the initial shuffling library. Interestingly, a de novo mutation, T111H, emerged in this round of DNA shuffling (Table 1 and FIG. 11). While T111 and R111 were dosed at a 1:1 ratio in the starting library, T111H was adapted by more than 50% of the survival clones (17 out of 32). Given that T111H is extremely rare in the starting library, the enrichment sends a strong signal that T111H is a critical mutation which underpins the current evolution landscape of TadA. We installed into TadA all mutations that significantly enriched in selection and obtained TadA-RA4.0-4.6 (Table 1). All mutants survive strongly in the bacterial selection assay, resulting in four orders of magnitude more survival clones on plates with 400-800 μg/ml Kanamycin (FIG. 11b).

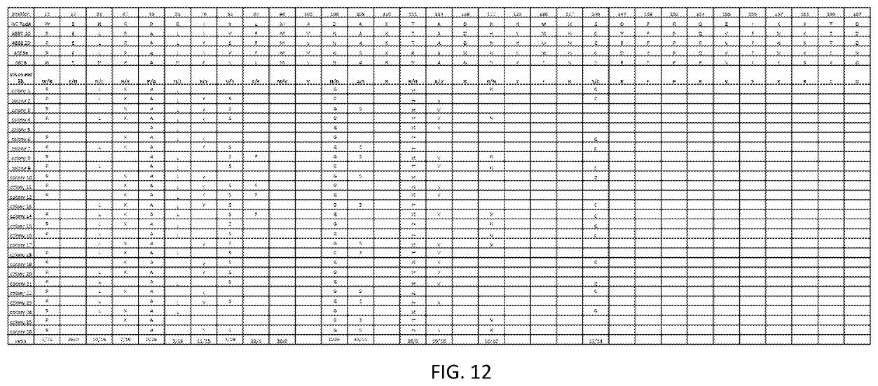

In the final round of DNA shuffling, we increased the selection stringency by forcing TadA to correct two premature stop codons (CA) and an active site mutation (TA) in Cam^{R}-R18*-R65*-H193Y, to maintain the high activity targeting YA sequences. In this round of shuffling, we fixed mutations that are strongly enriched in the 4^{th} round of selection and shuffled the mutations that are not covered in the 4^{th} round of selection and some neutral mutations in 4^{th} round of selection. W23R, H36L, R47K, P48A, R51L, V82S, D108G, T111H, A114V and S146C are strongly enriched in this round of selection and validation (FIG. 12). Incorporation of these beneficial mutations into TadA-RA4s brought us TadA-RA5.0-5.6 (Table 1). The final TadA variants combined mutations from TadA-RA3s, TadA7.10, TadA8.20, and TadA8e, indicating that mutations isolated from different sequence backgrounds and in different evolution trajectories can be compatible.

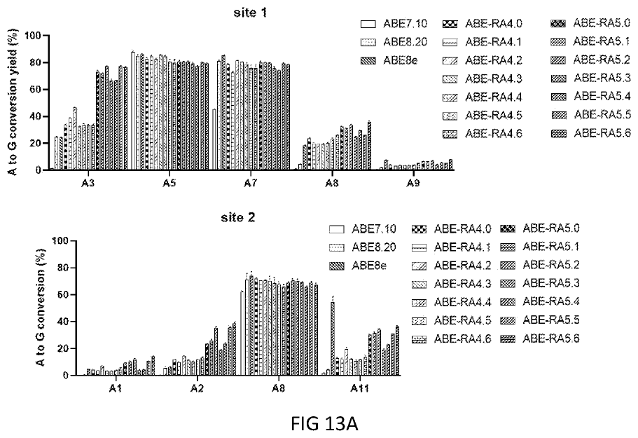

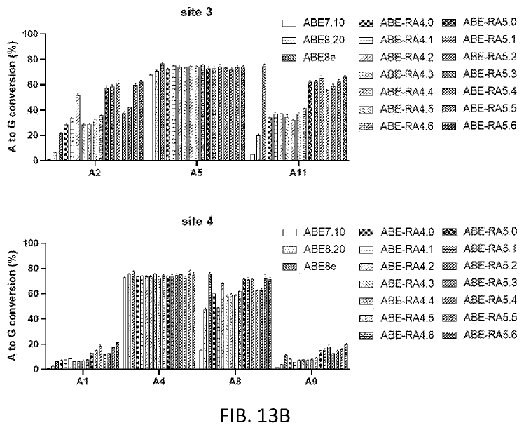

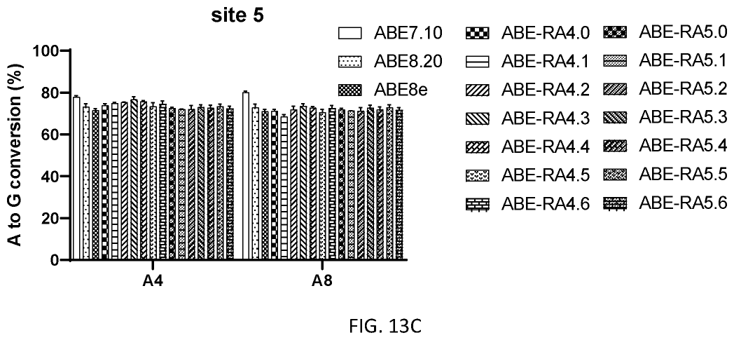

We directed these new ABEs to target sites 1-5 in HEK293T cells and compared them with the state-of-the-art ABEs: ABE7.10, ABE8.20 (22), and ABE8e (7). While outperforming ABE7.10 consistently, ABE-RA4s and ABE-RA5s generated equally strong editing as ABE8.20 and ABE8e, the two most active ABEs characterized to date, at positions 4-8 in the protospacer (FIG. 1c and FIG. 13). ABE-RA4s and ABE-RA5s generated 71.0-85.4% editing at positions 4-8, while ABE8.20 and ABE8e delivered 70.8-84.8% and 70.9-86.2% A:T-to-G:C editing at those positions (A8 in site 1 and 4 excluded). This observation is not surprising as base editing saturates in cooperative cell lines—the mutation rate in the strong editing window is limited by transfection efficiency rather than base editor activity (37). Specifically, A8 in site 1 and site 4 is preceded by G, wherein ABE-RA5s (31.2-33.5% at A8 of site 1, 71.1-71.3% at A8 of site 4) outperformed ABE8.20 (4.5% at A8 of site 1, 47.4% at A8 of site 4) and ABE8e (18.5% at A8 of site 1, 75.8% at A8 of site 4). We next analyzed protospacer positions beyond the canonical editing window. Satisfyingly, ABE-RA4s and ABE-RA5s are universally more active than ABE8.20, and ABE8e in editing positions spanning protospacer positions 1 and 3, and this effect is most evident with ABE-RA5.2, the best ABE variant we obtained in our evolution (FIG. 1d and FIG. 13). Specifically, ABE-RA5.2 edited AA₃ in site 1, and AA₂ in site 2, CA₂ in site 3 to 77.0±0.3%, 35.4±1.4%, and 61.4±1.7%, respectively, wherein editing of ABE7.10 was barely detectable (1.4±0.2%, 0.5±0.1%, 0.8±0.1%). Although ABE8.20 and ABE8e generated significant editing at these sites −24.6±0.5%, 5.5±0.9%, 6.3±0.5% for ABE8.20, and 24.4±0.3%, 6.2±0.3% and 21.5±0.8% for ABE8e, the editing levels are much lower than those delivered by ABE8r. Collectively, ABE-RA5.2 edits A at protospacer positions 1-3 at least 2.8-fold (up to 5.7-fold) more robustly than the most active ABEs developed to date.

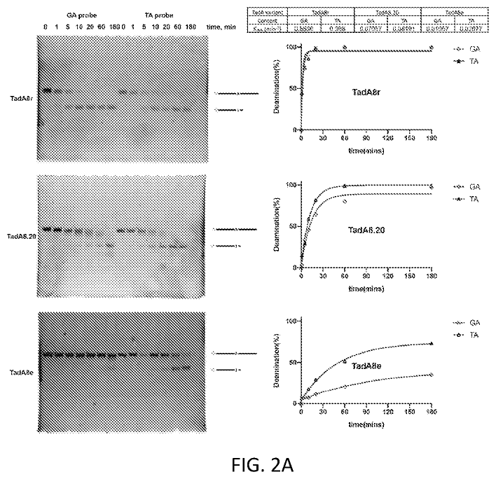

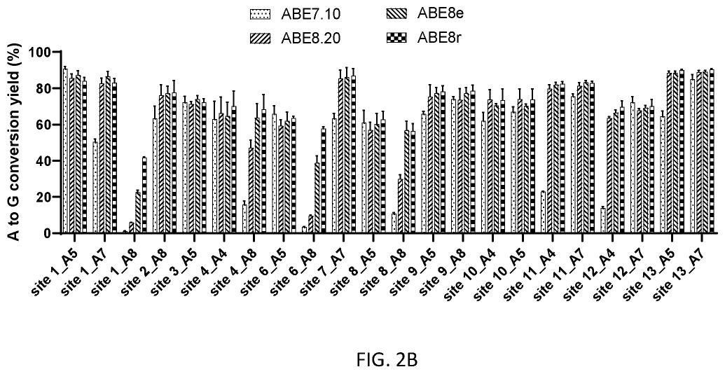

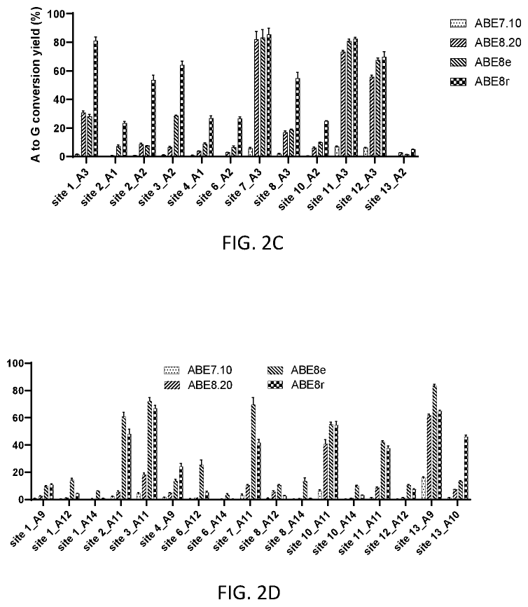

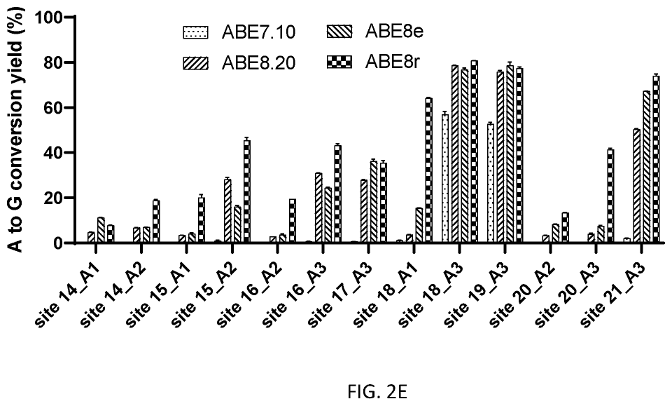

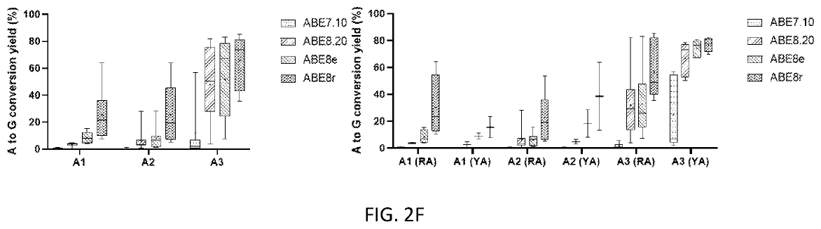

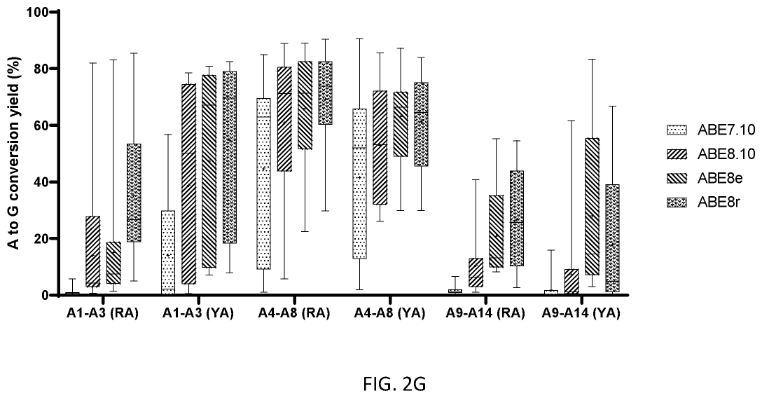

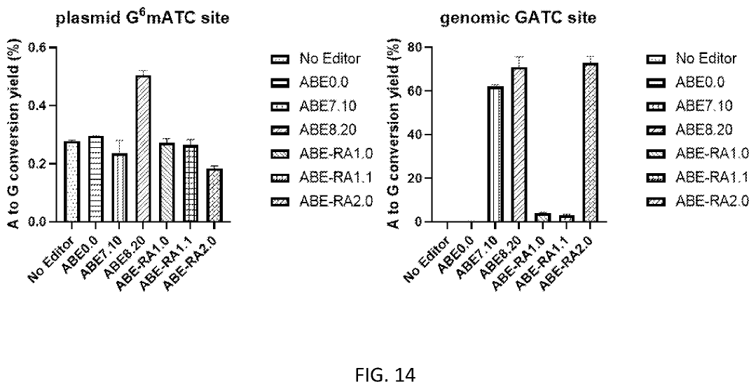

20 To test whether our de novo evolved mutations in TadA-RAs accept N⁶-methyldeoxyadenosine or not, we codelivered ABE-RA2.0, a sgRNA targeting a plasmid G⁶mATC site and a plasmid prepped from E. coli (G⁶mATC is proved to be fully methylated in E. coli) into HEK293T cells. ABE-RA2.0 failed to edit N⁶-methyldeoxyadenosine in a plasmid in HEK293T cells (FIG. 14), confirming that ABE-RA did not acquire activity on N⁶-methyldeoxyadenosine through directed evolution. Finally, we recoded our most advanced ABE, ABE-RA5.2, for mammalian expression and named it ABE8r for further characterization (FIG. 2).

### 3. Characterization of ABE8r in Human Cells

We compared adenine deamination efficiency of TadA8r in ssDNA with TadA8.20 and TadA8e. Maltose binding protein (MBP) fused TadA8r, TadA8.20, and TadA8e were purified through immobilized metal affinity chromatography. A Tobacco Etch Virus (TEV) protease cutting site was installed between MBP and TadA*. After TEV proteinase treatment, TadA8r, TadA8.20, and TadA8e were purified by immobilized metal affinity chromatography, ion-exchange chromatography, and size-exclusion chromatography. DNA deamination assays were carried out using 5′-radiolabeled ssDNA oligos under single-turnover conditions. A-to-I conversion was measured to determine the apparent first-order deamination rate constant (kₐₚₚ) (FIG. 2a). Both TadA8.20 and TadA8e preferred TA over GA (kₐₚₚ=0.07 min⁻¹ and 0.08 min⁻¹ for TadA 8.20 on GA and TA probes, respectively; kₐₚₚ=0.01 min⁻¹ and 0.02 min⁻¹ for TadA8e on GA and TA probes, respectively). The kₐₚₚ for TadA8r is much higher—0.55 min⁻¹ on the GA probe and 0.39 min⁻¹, on on the TA probe). These results suggest that TadA8r has much improved kinetics and altered context preferences compared with previously reported TadA variants.

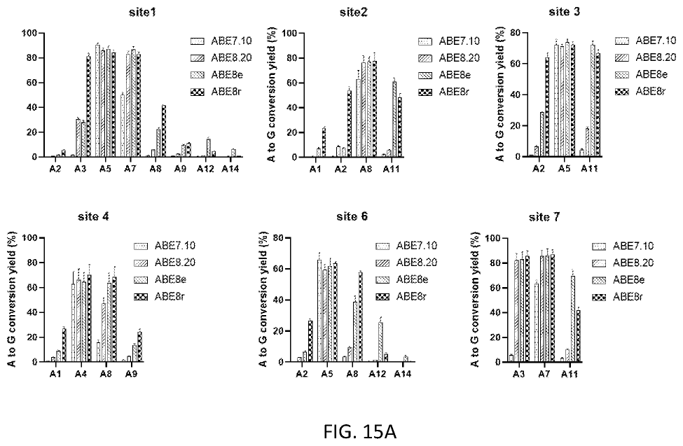

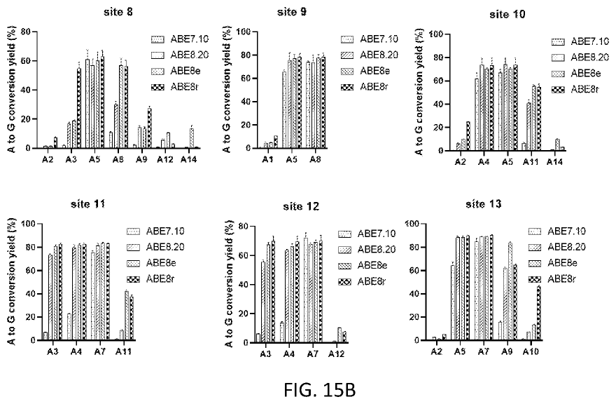

To further characterize ABE8r in mammalian cells, we chose sites with different bases proceeding and following the target A to systematically evaluate the context preference of ABE8r. When the target A situates at protospacer positions 4-8, ABE8r showed superior activity (41.7-90.3% editing among 12 genomic loci, FIG. 2b and FIG. 15). Although ABE8r consistently outperforms ABE7.10, especially at the edges of the strong editing window (protospacer positions 4 and 8), its activity is hardly differentiable with ABE8.20 and ABE8e at positions 4-8. ABE8r shows advantages over ABE8.20 and ABE8e at some A8 positions (site 1, site 4, site 6, and site 8). Since most protospacers contain more than one A, we extended our analysis to cover protospacer positions 1-14. Consistent with what was observed for ABE-RA4s and ABE-RA5s, ABE8r constantly generated much higher editing at protospacer positions 1-3, with the editing level at position 3 frequently approaching saturation (FIG. 2c and FIG. 15). Saturated editing levels are defined by maximum editing observed at protospacer positions 4-8 (˜80% in this study) and are typically limited by cell states and transfection efficiency. ABE8r results in 7-40-fold and 3-fold, 1.9-9.0-fold and 2.3-7.2-fold, 1.0-3.2-fold and 1.0-2.9-fold higher editing at A1, A2 and A3 positions than ABE8.20 and ABE8e, respectively. Trends at protospacer positions 9-14 are less consistent (FIG. 2d and FIG. 15). While still outperforming ABE8.20 in most cases, ABE8r is generally less efficient than ABE8e when editing A more adjacent to PAM, with the exception for some RA sequences. For example, ABE8r and ABE8e generated 5.2±0.9% and 25.3±3.7% editing at CA₁₂ of site 6, respectively (FIG. 2d). However, 46.1±1.0% and 13.2±0.6% editing was observed at AA₁₀ of site 13 for ABE8r and ABE8e. Whilst ABE8e constantly broadens the editing window with a bell-shape editing pattern, ABE8r has its activity more restricted at protospacer positions 9-14, a feature that may enable ABE8r to generate fewer bystander edits and purer editing outcomes.

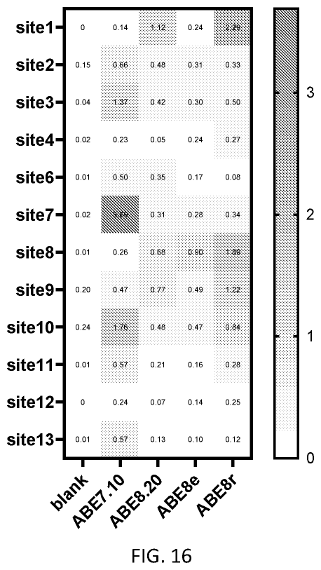

We analyzed indel levels generated by ABE7.10, ABE8.20, ABE8e, and ABE8r. ABE8r delivers indel levels comparable to ABE8.20 and ABE8e, suggesting that the increased deamination activity does not promote more double-stranded breaks in human cells (FIG. 16).

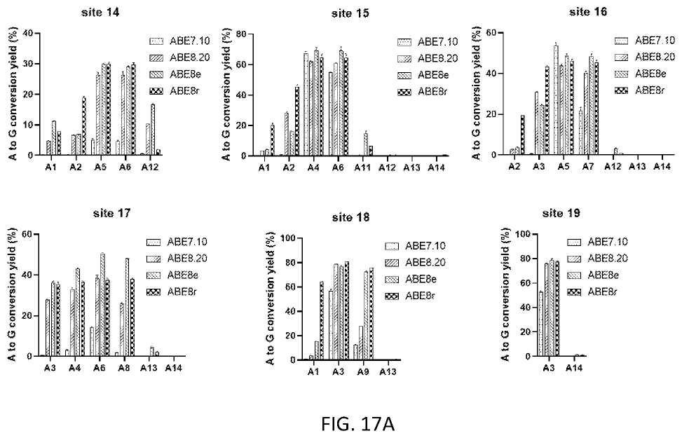

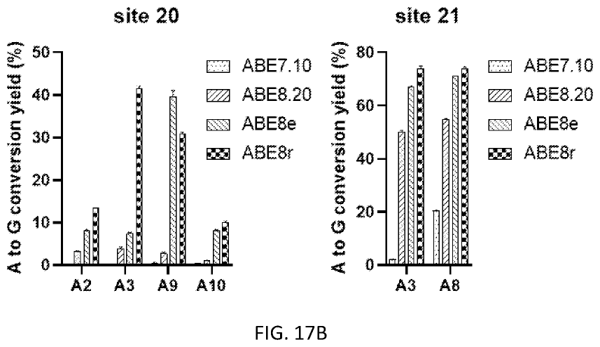

Motivated by the observation that ABE8 efficiently edits PAM distal positions, we included 8 additional target sites with A at protospacer positions 1-3. We confirmed that the observed trend held true with additional genomic loci (FIG. 2e and FIG. 17). Lastly, we summarized the performance of ABE8r at 20 genomic loci in different sequence contexts and compared with that of ABE7.10, ABE8.20, and ABE8e (FIG. 2f). ABE8r edited A at protospacer positions 1-3 to 28.1±20.1%, 29.9±19.2%, and 65.4±18.1%, respectively, whereas ABE7.10 remained mostly inactive at these positions. ABE8.20 and ABE8e accepted A at protospacer positions 1-3, albeit at a much lower level compared to ABE8r—3.2±1.5%, 7.6±7.8%, and 47.2±26.3% for ABE8.20, and 9.2±4.1%, 9.9±7.9%, and 51.2±27.7% for ABE8e, respectively. We further dissected activity based on sequence contexts. ABE8r outperforms ABE7.10, ABE8.20, and ABE8e for both RA and YA sites at protospacer positions 1-3 (FIG. 2f). While ABE8r remains more active than ABE7.10 and ABE8.20 at protospacer position 9-14, it succumbs to ABE8e in editing YA at these positions (FIG. 2g). Satisfyingly, as aimed by our directed evolution designs, ABE8r clearly wins all battles at RA sequences with a more visible margin when the target A is outside the most comfortable editing window. ABE8r, with its superior activity, also broadens the editing window on the PAM distal side, offering a broadened editing window that comfortably covers positions 3-8 in the protospacer.

### 4. Off-Target Activity of ABE8r

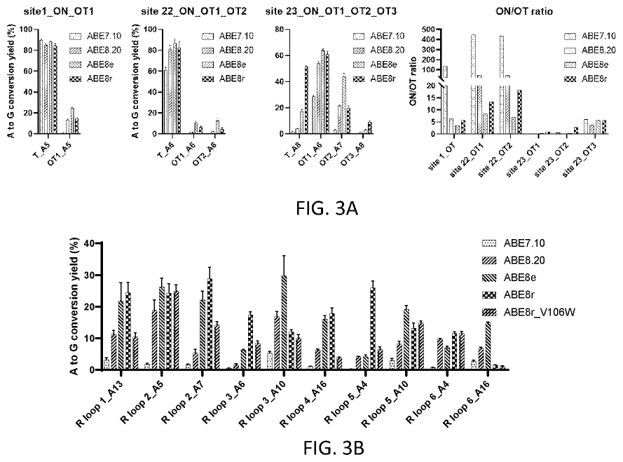

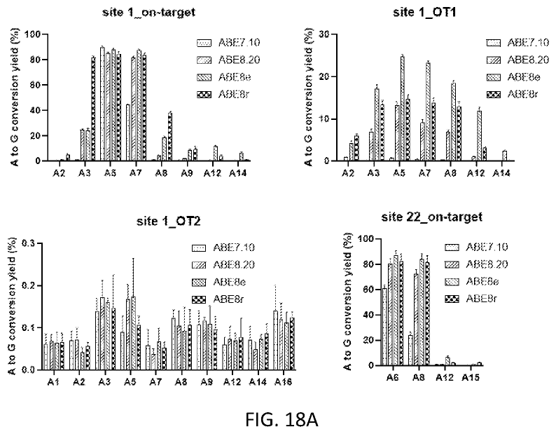

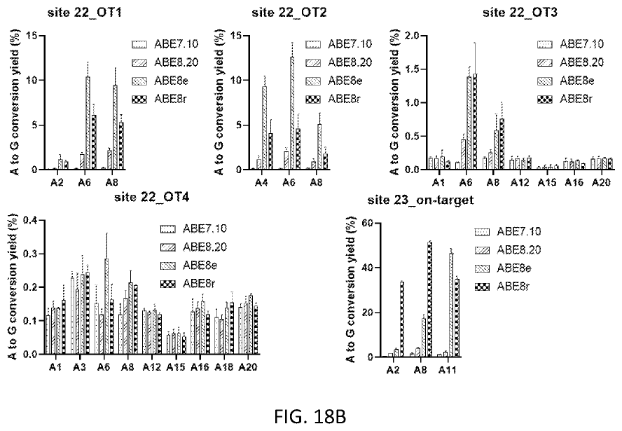

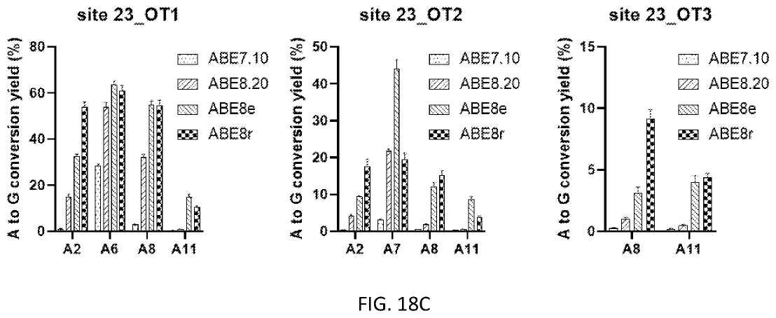

We next evaluated the off-target effects of ABE8r on DNA. Cas9-dependent off-target (OT) activity was analyzed for the top 2-3 OT sites for sites 1 (HEK2), site 22 (HEK3), and 23 (EMX1) identified through genome-wide, unbiased identification of DSBs enabled by sequencing (GUIDE-seq) (38) and in vitro identified genomic sequences susceptible to cleavage (CIRCLE-seq) (39). At OT site 1 of HEK2, ABE7.10, ABE8.20, ABE8e, and ABE8r generated 0.7%, 13.2%, 24.7%, and 14.7% A;T-to-G:C editing, respectively (FIG. 3a). We did not observe significant editing at OT site 2 of HEK2 except for ABE8e (0.2%), suggesting that Cas9-dependent off-target effects do not fully translate to adenine base editing, consistent with previous reports (3). ABE8r generated more obvious Cas9-dependent off-target editing than ABE7.10 (FIG. 18), which is not surprising given its superior DNA-editing activity. Nevertheless, ABE8r produced Cas9-dependent off-target editing at levels comparable to ABE8.20 and much lower than ABE8e. The on-target editing to off-target editing ratios for ABE8r are higher than ABE8e across 8 off-target sites (FIG. 3a, right). Note that the RA preference of ABE8r extends to its off-target editing activity. For example, with overall lower off-target editing observed at HEK2 OT site 1, ABE8r generated 6.1% editing at GA₂, while ABE8e generated 4.1% editing at GA₂. Similar observations were obtained at GA₂ of site 23 OT 1 (FIG. 18).

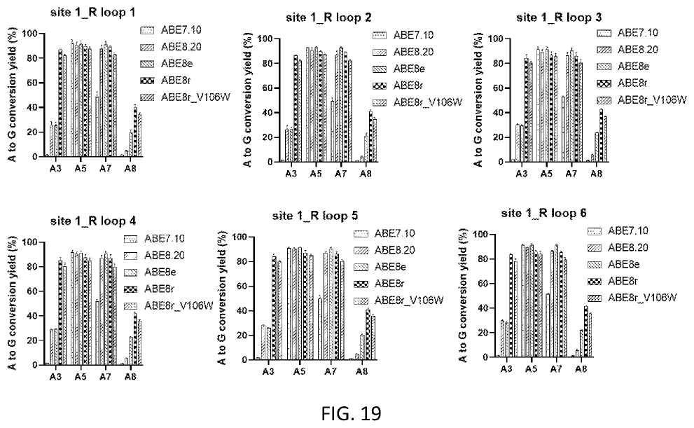

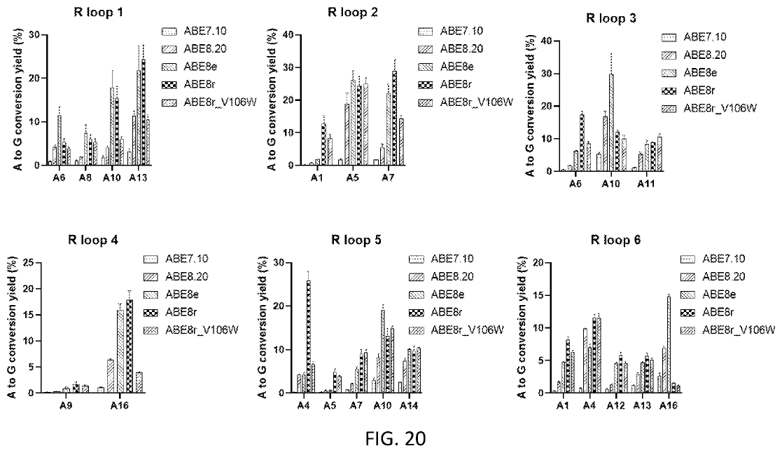

To examine Cas9-independent off-target activity of ABE8r, we adapted an orthogonal R-loop assay previously developed to evaluate genome-wide off-target effects of base editors (40, 41). ABEs were codelivered with a sgRNA targeting site 1. A catalytically inactive SaCas9 (dSaCas9) was delivered to target Sa sites 1-6 to present a constant R loop. Editing at these R loops serves as a surrogate for Cas9-independent off-target activity. On-target activity remained consistent for all ABEs in the presence of dSaCas9 (FIG. 19). ABE8r generated more off-target editing than ABE7.10 at dSaCas9-targeted loci (FIG. 20). Off-target editing generated by ABE8r is mostly comparable to that of ABE8.20, but lower than that of ABE8e. For example, ABE8r produced 12% off-target editing at A3 of R loop 3, compared to 29.8% by ABE8e (FIG. 3b). Introduction of fidelity-improving mutations into evolved TadA variants has been demonstrated to reduce off-target editing by adenine base editors(31, 36). We installed a previously reported mutation, V106W, into ABE8r and obtained ABE8r-A106W. ABE8r-A106W shows markedly lower off-target editing compared to ABE8r (FIG. 3b). For example, ABE8r-A106W generated 3.9% editing at A16 in R loop 4 and 6.6% editing at A4 in R loop 5, while ABE8r delivers 17.8% and 25.9% editing at these positions (FIG. 3b).

5. Compatibility of TadA8r with Different CRISPR Effector Proteins

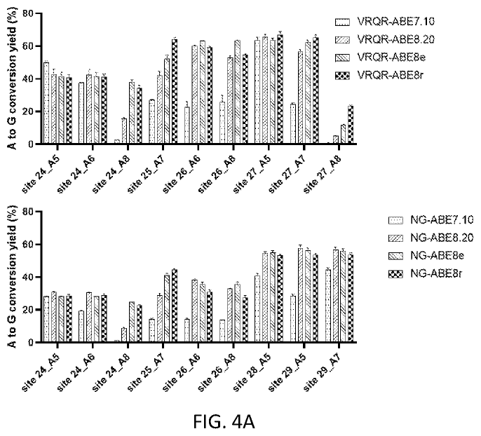

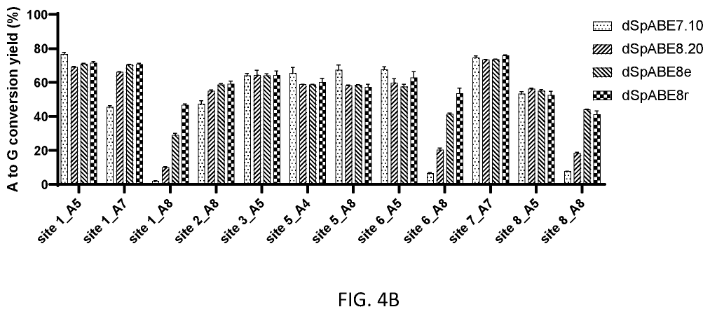

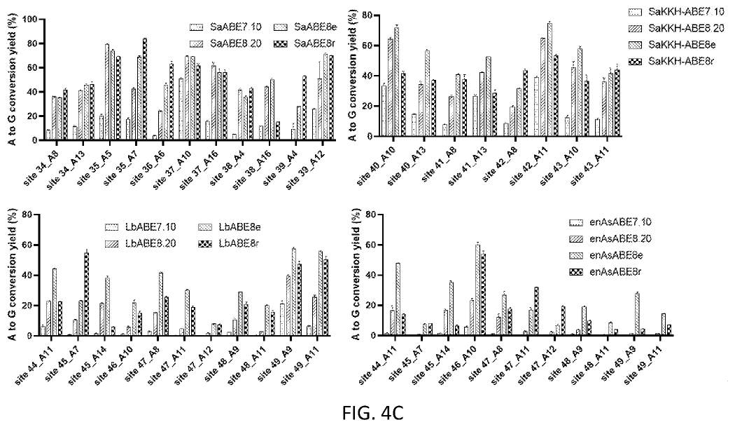

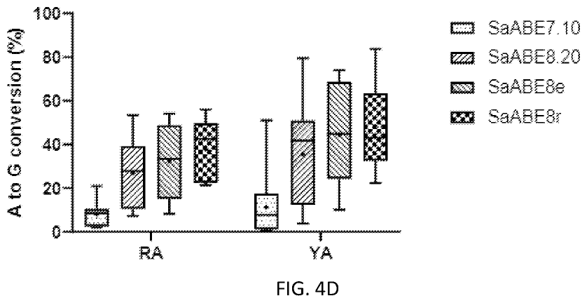

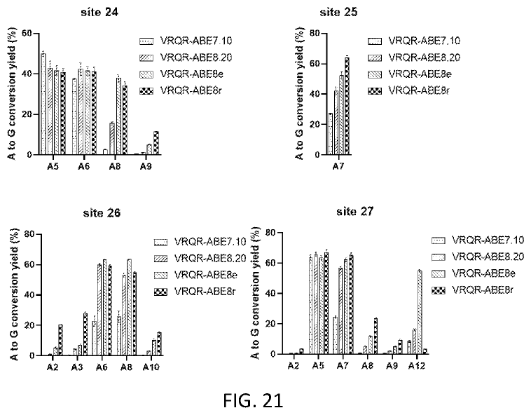

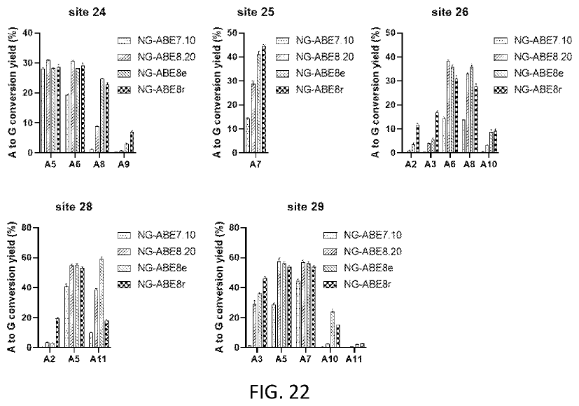

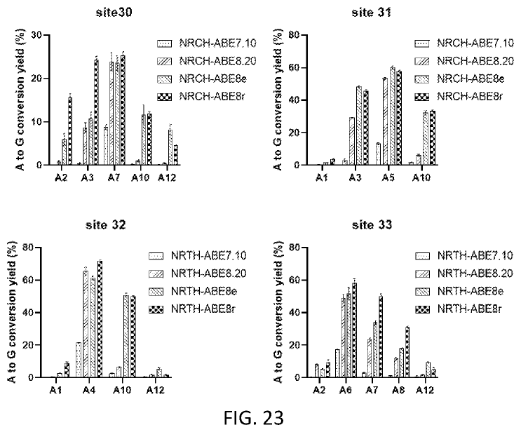

To expand the target scope, we constructed ABE8r variants by replacing SpCas9 with variants of high specificity or altered and broadened PAM specificities, including SpCas9-VRQR (42), SpCas9-NG (25), SpCas9-NRCH (26), and SpCas9-NRTH (26). TadA8r is broadly compatible with these SpCas9 variants, generating 41.2-67.0%, 29.0-53.7%, 25.2-57.8%, and 58.1-71.6% editing at the most strongly edited A in the protospacer with SpCas9-VRQR (42) (FIG. 4a and FIG. 21), SpCas9-NG (25) (FIG. 4a and FIG. 22), SpCas9-NRCH (26) and SpCas9-NRTH (26) (FIG. 4a and FIG. 23), respectively. The overall activity of TadA8r coupled with these SpCas9 variants is higher than, or comparable to, TadA8.20 and TadA8e derivatives. Importantly, the preference of ABE8r for PAM-distal positions and RA sequences persists. For example, editing at CA₂, AA₃ at site 26, GA₂ at site 28, and CA₂, AA₃ at site 30 was higher with TadA8r derivatives than TadA8.20 and TadA8e derivatives.

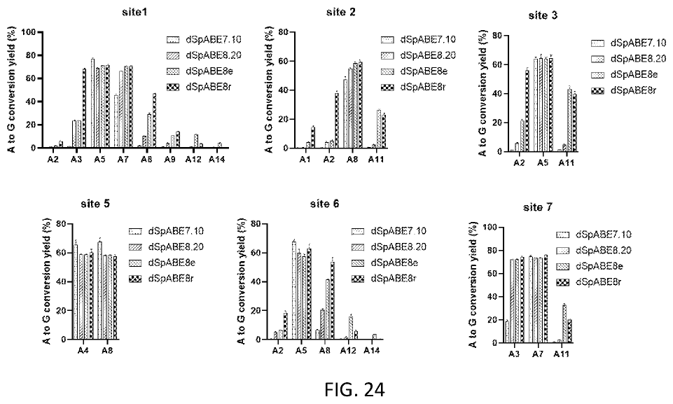

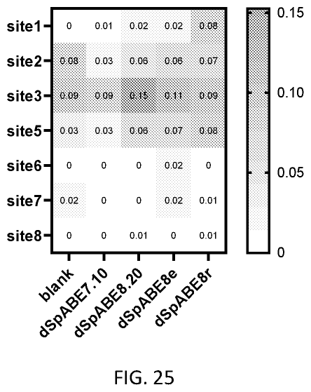

Indels are frequently observed as side products of base editing when highly active deaminases are fused to Cas9 nickase, as simultaneous deamination and nicking may result in double-stranded breaks, likely through an abasic site intermediate (7, 43). To reduce incidents of indels, we constructed an ABE8r variant in which nCas9 was replaced with dCas9 (FIG. 4b and FIG. 24). Editing activity remained high even when the target strand was no longer nicked, suggesting that superior deamination efficiency may surpass preferences of cellular repair machinery for adenine base editing. Importantly, with dCas9 serving as the DNA engaging module, indel formation was reduced to the background level (FIG. 25).

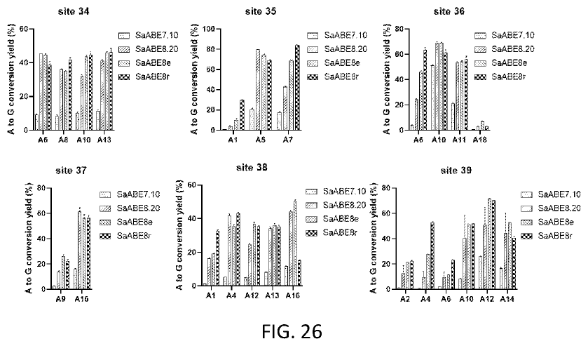

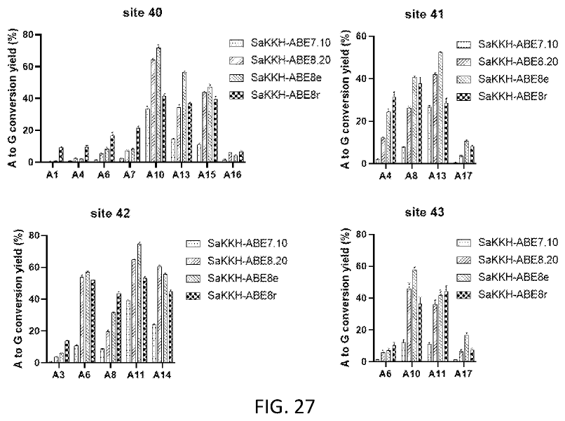

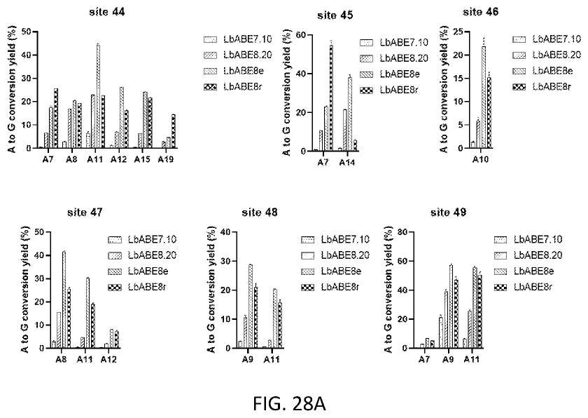

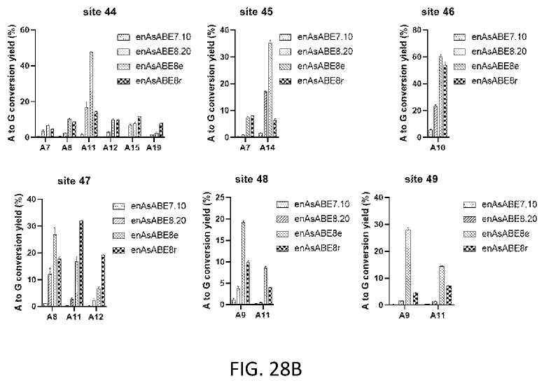

To further increase the application scope, we fused TadA8r to additional CRISPR effector proteins, including SaCas9 (27, 28), SaKKHCas9 (28), LbCas12a (29), and enAsCas12a (29, 30), and characterized these new ABEs in HEK293T cells. Note that no nickase mutations are known for Cas12a. We therefore directly employed nuclease-deficient Cas12a (dCas12a) in LbABE8r and enAsABE8r. We tested 4-6 sites for each new ABE. TadA8r is broadly compatible with these CRISPR effector proteins, generating 15.1-83.7%, 28.5-53.2%, 5.8-54.7%, and 4.0-53.9% editing in forms of SaABE8r, SaKKHABE8r, LbABE8r, and enAsaBE8r, respectively (FIG. 4c and FIG. 26-28). The editing levels are comparable with those produced by SaABE8e, SaKKHABE8e, LbABE8e, and enAsABE8e, and are much higher than ABEs derived from TadA7.10, which is known to be less compatible with non-SpCas9 CRISPR systems (6). As expected, the editing windows are altered when different CRISPR effector proteins are employed (FIG. 26-28). SaABE8r and SaKKHABE8r edit A efficiently at protospacer positions 3-16, whereas LbABE8r and enAsaBE8r edit A at positions 7-15, respectively. These results are consistent with the editing windows proposed for corresponding cytosine base editors (44, 45) and ABE8e (7). SaABE8r and SaKKHABE8r prefer RA sequence and positions distal to the PAM. For example, SaABE8r and SaKKHABE8r show 1.4-2.9-fold and 1.6-7.6-fold higher editing at site 35 (A1), site 36 (A6), site 38 (A1), site 39 (A4), site 40 (A1, A4, A6 and A7), site 41 (A4) and site 42 (A3) than corresponding ABE8.20 and ABE8e derivatives.

Finally, we analyzed 23 target As edited by SaABE8r, and SaKKH-ABE8r to more than 20% and plotted bulk editing efficiencies at RA and YA sequences (FIG. 4d). TadA8r clearly outperforms Tad8e at RA sequences. Collectively, as a highly active deoxyadenosine deaminase, TadA8r is broadly compatible with CRISPR proteins with a preference for RA sequences.

### 6. Application of ABE8r in Correcting Disease-Relevant Mutations

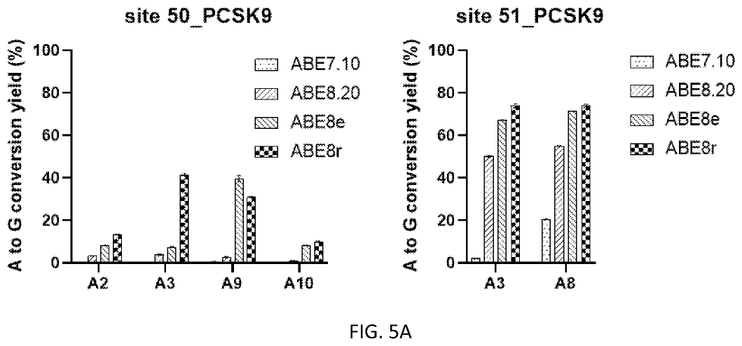

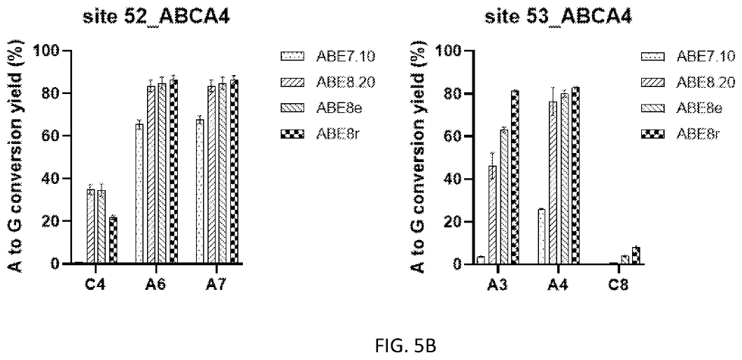

We applied ABE8r to correct disease-causing/associated mutations in human cells. We first applied ABE8r to edit PCSK9 (proprotein convertase subtilisin/kexin type 9), which is mainly expressed in the liver and acts as a negative regulator of low-density lipoprotein (LDL) receptor (46). Loss of function mutations in PCSK9 can lower the level of LDL cholesterol in blood thus presenting a promising approach for reducing the risk of atherosclerotic cardiovascular disease. ABEmax and ABE8.8 have been applied to edit the splicing sites in PCSK9 in vivo (47, 48). We tested ABE7.10, ABE8.20, ABE8e, and ABE8r to edit two splicing sites (A3 of site 42 and A3 of site 43) of PCSK9. We chose these two target sites because the corresponding sgRNAs were predicted to have less DNA off-target effects (47) (FIG. 5a). ABE8r generated 41.4±0.6% editing at site 42, 5.8-fold higher than that of ABE8e (7.4±0.3%). ABE7.10 had no detectable editing at this site, and ABE8.20 gave 3.9±0.3% editing. ABE8r also outperforms ABE7.10, ABE8.20, ABE8e at site 43.

We next applied ABE8r to correct a G:C-to-A:T mutation in ABCA4. The G:C-to-A:T mutation creates a Gly1961Glu mutation that is known to be associated with inherited retinal disease (49). Two sgRNAs were designed to correct this mutation (A6 of site 44 and A3 of site 45). Although all editors generated high editing (83.5%, 84.7%, and 86.3%) when at A6 in site 44, ABE8.20 and ABE8e showed bystander editing at C4 higher than ABE8r(34.9%, 34.6%, and 21.8% for ABE8.20, ABE8e, and ABE8r) (FIG. 5b). ABE8r delivered 81.3% editing at A3 of site 45, while ABE8.20 and ABE8e showed much lower editing, 46.2% and 63.2%. ABE7.10 was barely active at this site, delivering 3.6% A:T-to-G:C editing (FIG. 5b).

These results, taken together, showcase the therapeutic potential of ABE8r, especially for PAM-distal As and RAs, which can be challenging targets for available base editors.

### B. Discussion

Three rounds of de novo directed evolution and two rounds of DNA shuffling brought us ABE8r, a new adenine base editor with improved editing efficiency and altered context preferences. TadA8r is 6.86-fold and 54-fold faster in deaminating GA in ssDNA than TadA8.20 and TadA8e, respectively.

ABE8r shoes Cas9-dependent and Cas9-independent DNA off-target editing comparable to ABE8.20, but lower than ABE8e.

TadA8r is compatible with a suite of effector proteins, including engineered SpCas9s with expanded PAM sequences (SpCas9-VRQR, SpCas9-NG, SpCas9-NRCH and SpCas9-NRTH), SaCas9, SaKKHCas9, LbCpf, and enAsCpf, thereby may deliver A:T-to-G:C editing to sites that are challenging for SpCas9. Replacement of SpCas9 nickase with dSpCas9 in ABE8r reduces the indel levels while maintaining on-target editing efficiencies.

We evaluated ABE8r on two disease relevant loci, PCSK9 and ABCA4. Our results support the therapeutic potential of ABE8r, a new adenine base editor with features complementary to existing adenine base editors.

In addition to ABE8r, we identified ABE-RA2.0, 2.1 and ABE-RA3.0, 3.1, 3.2, 3.3, which delivers robust editing to GA sequences at positions 4-8, but loses activity outside the strong editing window. These editors may therefore be more specific and generate purer editing outcomes.

In summary, ABE8r is a new adenine base editor of improved activity, altered context preferences, shifted editing windows, and high specificity.

### C. General Methods.

DNA amplification was conducted by PCR using Phusion™ High-Fidelity DNA Polymerase (Fisher Scientific, F530L), Phusion U Hot Start DNA Polymerase (Fisher Scientific, F555S) or Taq DNA Polymerase (New England BioLabs, M0273X) unless otherwise noted. All the bacterial and mammalian cell editor plasmids were assembled using Golden Gate Cloning. Selection plasmids and sgRNA constructs were assembled by either user cloning or quick exchange. Starting templates for PCR were either purchased from Addgene or bacterial or mammalian codon-optimized gBlock Gene Fragments by Integrated DNA Technologies. All the primers used for user assembly of sgRNA constructs were listed in (Supplementary Table 1). All editor constructs, selection constructs, sgRNA constructs were transformed with DH5a competent cells. All plasmids were purified by QIAprep Spin Miniprep Kit (Qiagen).

### 1. Generation of Editor Libraries for Directed Evolution.

Libraries of editor constructs were generated by two-piece Golden Gate assembly of a TadA* PCR product and an acceptor plasmid containing the backbone of the editor construct (sgRNA was pre-installed) using restriction enzyme BsaI. All editor plasmids are composed of an SC101 origin of replication, a β-lactamase gene for plasmid maintenance with Ampicillin, a PBAD promoter driving TadA*-dCas9 expression, and a lac promoter driving sgRNA transcription. The architecture of the base editors used during bacterial selection is: TadA*-linker (32 aa)-dCas9. As in different rounds of selection different sgRNAs would be used, we designed a two-dropout golden gate acceptor, in which mRFP was for installation of TadA* using restriction enzyme BsaI, mcherry was for installation of sgRNA using restriction enzyme BsmBI. Before making editor libraries for each round of selection, a sgRNA was pre-installed to form the acceptor plasmid which was used in library construction.

TadA* PCR product in selection rounds 1-3 were generated by error prone PCR of TadA variant templates (Supplementary Table 2) using GeneMorph II Random Mutagenesis Kit (Agilent, 200550) following the manufacturer's protocol. Specifically, 2 μg DNA template (˜125 ng TadA* gene), 800 μM dNTP mix (200 uM each), 0.5 μM forward primer YX209, 0.5 μM reverse primer YX210, 1.25 U Mutazyme II DNA polymerase, 1× Mutazyme II reaction buffer were used for 25 μl PCR reaction using the following program: 95° C., 2 min; 30 cycles of (95° C., 30 s; 60° C., 30 s; 72° C., 1 min); 72° C., 10 min. Mutation rate was about 1-3 mutations/500 bp. The PCR product was purified by gel electrophoresis using a 1% agarose gel and QIAquick Gel Extraction Kit (Qiagen).

TadA* PCR product in selection rounds 4 and 5 were generated by overlapping PCR of several TadA* fragments. Mutations were incorporated either by synthetic DNA oligos or manually mixing PCR templates or primers which contains the mutations to be shuffled in 1:1 ratio. Specifically, TadA* library for the 4^{th} round selection (1^{st} round DNA shuffling) was generated by overlapping PCR of DNA fragments 1A, 1B and 1C (Supplementary Table 3). Fragment 1A was generated by amplification of DNA templates containing manually mixed TadA_R51(R/H) (1:1) with fixed P48A using primers YX201 and WT1681, mutation I76(I/F) was incorporated in primer WT1681. Fragment 1b was generated by amplification of ultramers WT1675/WT1676 (1:1) using primers WT1679/WT1680 (1:1) as forward primer and WT1682 as reverse primer. Mutation L84(L/F) was incorporated in primers WT1679/WT1680, mutations A106(A/V), K110(K/R), T111(T/R), D119(D/N), H122(H/R), H123(H/Y), M126(M/I) and N127(N/K) were incorporated in ultramers WT1675/WT1676 using mixed bases by synthesis. Fragment 1C was generated by amplification of ultramers WT1677/WT1678 (1:1) using primers WT1683 and YX210. Mutations S146(S/C), D147(D/R), F149(F/Y), R152(R/P), Q154(Q/R), E155(E/V), I156(I/F), K157(K/N), K161(K/N), T166(T/I) and D167(D/N) were incorporated in ultramers. After amplification, PCR fragments were gel purified by QIAquick Gel Extraction Kit (Qiagen), applied for overlapping PCR. 200 ng 1A, 140 ng 1B and 100 ng 1C were used to set up 100 ul PCR reaction using Phusion DNA polymerase following the program: 98° C., 3 min; 15 cycles of (98° C., 30 s; 55° C., 30 s; 72° C., 30 s); 75° C. 5 min, then 0.5 μM primers YX209 and YX210 were added to the system and followed by an extra 10 cycles of amplification using 60° C. as annealing temperature. The PCR product was gel purified by QIAquick Gel Extraction Kit (Qiagen). The DNA shuffling for TadA* library for 5^{th} round of selection was similar with that of 4^{th} round TadA* library, DNA fragments 2A, 2B, 2C, 2D and 2E were used for overlapping PCR (Supplementary Table 3). Sequences of DNA oligos used for generation of TadA* libraries and sequencing (Supplementary Table 4).

Editor libraries were assembled by Golden Gate assembly using the following conditions: 2 μg acceptor plasmid, 600 ng TadA* library insert, 200 U BsaI-HF® v2 (New England BioLabs, R3733S), 30 U T4 ligase (Promega, M1801) and 1×T4 ligase buffer in 200 μl reaction were incubated at 37° C. for 24 h, the enzymes were deactivated at 65° C. for 20 min. Assembled editor libraries were purified by QIAquick PCR Purification Kit (Qiagen), eluted with 20 μl H₂O. 15 μl of the eluted product was added into 50 μl NEB® 10-beta electrocompetent E. coli and electroporated with MicroPulser Electroporator (Bio-Rad) using bacteria program. Typically, one electroporation can generate 5-10 million colony forming units (c.f.u.). Electroporated cells were recovered in 10 ml pre-warmed NEB® 10-beta/Stable Outgrowth Medium at 37° C. with shaking for 1 h, then added with 100 ml LB medium (Luria-Bertani medium) and 100 ul/ml ampicillin for bacteria maintenance and cultured for another 16 h before plasmid miniprep (Qiagen).

### 2. Directed Evolution for TadA* Variants

5 μg of editor library plasmid were mixed with 500 μl of home-made electrocompetent S1030 cells containing corresponding selection plasmid, electroporated with MicroPulser Electroporator (Bio-Rad) using bacteria program (50 ul×10 times electroporation). Typically, this round of electroporation can generate 50-100 million colony forming units (c.f.u.). Electroporated S1030 cells were recovered in 50 ml 2×YT medium with 20 mM glucose at 37° C. with shaking for 1 h, then added with 50 ml LB medium and 100 μg/ml ampicillin, corresponding antibiotics for selection plasmid maintenance and 1 mM arabinose to induce overexpression of editor proteins, then cultured for another 16 h to saturation. 2 ml of the saturated culture were plated onto each of 245 mm×245 mm square bioassay dishes containing 1.5% agar-LB, 100 μg/ml ampicillin, 50 μg/ml selection plasmid maintenance antibiotics, and a concentration of the selection antibiotic (Supplementary Table 5), plates were incubated at 37° C. for 24 h. 8-16 survived colonies were isolated, TadA* gene was amplified using primers WT022 and YX140 and submitted for sanger sequencing. All the survived colonies were scraped off the plates and editor library plasmids were isolated by QIAprep Spin Miniprep Kit (Qiagen), TadA* gene was amplified using primers YX209 and YX210, then subcloned with editor backbone acceptor. The survived library was transformed with electrocompetent S1030 cells (containing selection plasmid), the bacteria were induced, cultured and rechallenged on selection plates as above. Next, 16-32 survived colonies were isolated, TadA* gene was amplified using primers WT022 and YX140, and then submitted for Sanger sequencing. Mutations enriched in both selection and validation were cloned to mammalian ABE constructs and tested in HEK293T cells.

### 3. Bacteria Tittering Assay

100 ng editor plasmid was transformed into 50 μl chemical competent S1030 cells which contains the targeting selection plasmid. The S1030 cells were recovered in 1 ml LB medium at 37° C. with shaking for 1 h, then another 1 ml LB medium, 100 μg/ml Ampicillin, 50 g/ml antibiotics for selection plasmid maintenance, 1 mM arabinose were added to the bacterial culture. The culture was incubated at 37° C. with shaking for another 16 h to saturation. The bacterial culture was serial diluted with LB medium at tenfold intervals in total 5 times. Then, 4 μl of each bacterial culture in different concentrations were spotted onto bioassay dishes containing 1.5% agar-LB, 100 μg/ml ampicillin, 50 μg/ml selection plasmid maintenance antibiotics, and a concentration of the selection antibiotic. The plates were incubated at 37° C. for 24 h.

4. Preparation of A- and N⁶-Methyl-A Bearing E. coli tRNA^{Arg}(CGT) Probes

Unmethylated and methylated E. coli tRNA^{Arg}(CGT), tRNA #1, and tRNA #2 were synthesized by in vitro transcription using T7 RNA polymerase. ATP and N⁶-methyl-ATP (TriLink, N-1013) were supplied in the presence of UTP, CTP, and GTP to synthesize unmethylated and methylated RNA, respectively. RNA was purified by E.Z.N.A Micro RNA kits (Omega Bio-Tek, R7034) and quantified by NanoDrop One (Thermo Fisher Scientific). 5. In vitro deamination assays of wildtype TadA and TadA7.10 on E. coli tRNA^{Arg}(CGT) probes and RT-PCR

RNA was always preheated to 95° C. for 3 min and immediately cooled down before use. 200 ng E. coli tRNA #1 or tRNA #2 and 100 nM wildtype TadA or TadA7.10 were incubated in deamination buffer (50 mM Tris, 25 mM KCl, 2.5 mM MgCl₂, 2 mM dithiothreitol, and 10% (v/v) glycerol; pH 7.5) in the presence of 10 U SUPERase⋅In™ RNase Inhibitor (Thermo Fisher Scientific, AM2694) at 37° C. for 1 h. Reactions were quenched by incubating at 95° C. for 10 min. To convert tRNA into cDNA for sequencing, 2 μl reaction mixture was aliquoted and mixed with 0.5 μl of 50 μM reverse transcription primer. Primer annealing was enabled by heating up the mixture to 95° C. for 3 min, cooling down at a ramping rate of 2° C./s, and incubation at 25° C. for 2 min. To the reaction, 0.5 μL of GoScript reverse transcriptase (Promega, A5003) was added together with 2 μL of 5×GoScript RT buffer, 1 μL of 25 mM MgCl₂, 0.5 μL of 10 mM dNTPs, and 3.5 μL nuclease-free H₂O. The reverse transcription reaction was incubated at 42° C. for 1 h and then quenched at 65° C. for 20 min. 1 ul of reverse transcription reaction mixture was used as template for PCR reactions. The PCR follow the program: 95° C. for 3 min; 30 cycles of amplification (denaturing at 95° C. for 10 s, annealing at 60° C. for 10 s followed by extension at 72° C. for 20 s); and final extension at 72° C. for 5 min. sequence of E. coli tRNA, oligos used for reverse transcription and PCR are listed in Supplementary Table 6.

### 6. Single Turnover In Vitro DNA Deamination Assays of TadA8r, TadA8.20 and TadA8e on GA/TA Probes.

The single turnover DNA deamination reactions containing 4 uM TadA variants in deamination buffer (50 mM Tris, 25 mM KCl, 2.5 mM MgCl₂, 2 mM dithiothreitol, and 10% (v/v) glycerol; pH 7.5) and 5′ Fluorescein labeled ssDNA (IDT) (Supplementary Table 6) to a final concentration of 200 nM. All reactions were incubated at 37° C. At various time points (0, 1, 5, 10, 20, 60, 180 mins), 10 uL reaction mixture were aliquoted and quenched by adding 10 ul of hot water and incubating at 95° C. for 10 min. Reaction mixtures were supplied with 100 ug/ml Proteinase K (Fisher scientific) and incubated at 55° C. for 3 h followed by inactivating at 85° C. for 30 mins and 95° C. for 15 mins. To detected adenosine deamination, reaction mixture was incubated with 10 unit of E. coli EndonucleaseV in 1×NEB4 buffer at 37° C. for 1 h. After cleavage by EndoV, samples were mixed with 2-fold PAGE gel loading buffer (95% formamide, 10 mM EDTA, 0.025% SDS), heated at 95° C. for 5 min, resolved on 15% (v/v) denaturing polyacrylamide gel. Uncleavage substrate and cleavage product were visualized by ChemiDoc XRS+(Bio-rad) under fluorescein channel. DNA band quantification were analyzed using ImageJ Software. Curve fitting was done in GraphPad.

### 7. Cell Culture Conditions

HEK293T was purchased from ATCC and cultured in Dulbecco's modified Eagle's medium (DMEM) (Corning, 10-013-CV) supplemented with 10% (v/v) fetal bovine serum (FBS). HEK293T_ABCA4_G1961E stable cell line was generated by prime editing. Briefly, HEK293T cells in 96-well plate were transfected with 200 ng of PE2 editor plasmid and 80 ng of pegRNA plasmid by 0.5 ul of Lipofectamine 2000. After culturing for 3 days, cells were treated with 20 ul of trypsin at 37° C. for 3 min and then diluted with DMEM medium supplemented with 10% FBS. Cells were plated onto 96-well poly-d-Lysine-coated plates making 0-1 cells per well, cultured for 3-4 weeks, monoclonals were isolated. The targeting ABCA4 gene was amplified and sequenced by Sanger sequencing. Correct HEK293T_ABCA4_G1961E stable cell line was maintained in DMEM supplemented with 10% (v/v) FBS.

### 8. HEK293T Plasmid Transfection and Genomic DNA Preparation

HEK293T cells were seeded onto 96-well poly-d-Lysine-coated plates (Corning) at a density of 1×10⁴ cells per well. After 16-24 h, cells were transfected at approximately 70-80% confluency. 200 ng editor plasmid and 40 ng sgRNA plasmid were diluted to 25 μl total volume in Opti-MEM reduced serum medium (Gibco). The solution was mixed with 0.5 μl of Lipofectamine 2000 (Thermo Fisher Scientific) in 25 μl of Opti-MEM reduced serum medium and was incubated at room temperature for 20 min. The 50 μl mixture was then transferred to the Hek293T cells. Cells were cultured for 3 days. Medium was removed and cells were washed with 100 ul 1×PBS buffer (Corning), then 40 ul freshly prepared lysis buffer (100 mM Tris-HCl, pH 8.0, 0.05% SDS, 25 μg/ml Proteinase K (Thermo Fisher Scientific)) was added into each well. 96-well plates with lysis buffer were incubated at 37° C. for 30 min, then the lysates were transformed into 96-well PCR plates and incubated following the program: 55° C., 1 h; 85° C., 30 min; 95° C., 10 min.

### 9. Orthogonal R-Loop Assay

HEK293T cells were seeded onto 96-well poly-d-Lysine-coated plates (Corning) at a density of 1×10⁴ cells per well. After 16-24 h, cells were transfected at approximately 70-80% confluency. 40 ng of SpCas9 sgRNA plasmid, 40 ng of SaCas9 sgRNA plasmid, 150 ng of base editor plasmid and 150 ng of dSaCas9 plasmid were cotransfected into HEK293T cells using 0.5 μl of Lipofectamine 2000. Specifically, all plasmid DNA were mixed with Opti-MEM reduced serum medium in total volume 25 ul. The solution was mixed with 0.5 μl of Lipofectamine 2000 in 25 μl of Opti-MEM reduced serum medium and was incubated at room temperature for 20 min. The 50 μl mixture was then transferred to the Hek293T cells. Cells were cultured for 3 d, then washed with 1×PBS, followed by genomic DNA extraction by addition of 40 μl of freshly prepared lysis buffer (10 mM Tris-HCl, pH 8.0, 0.05% SDS, 25 μg/ml proteinase K directly into each transfected well. The mixture was incubated at 37° C. for 30 min, then the lysates were transformed into 96-well PCR plates and incubated following the program: 55° C., 1 h; 85° C., 30 min; 95° C., 10 min.

### 10. Next Generation Sequencing of Genomic DNA Samples

Genomic DNA of interests were amplified by two rounds of PCR. In the 1^{st} round PCR, genomic DNA was amplified with site specific Illumina primers (containing amplicon specific annealing part and Illumina adapter part) (All the Illumina primer pairs were listed in Supplementary Table 7). Briefly, 1 ul of cell lysate was added into 20 ul PCR reaction containing 1× Standard Taq reaction buffer, 800 uM dNTP mix (200 uM each), 0.5 uM forward primer, 0.5 uM reverse primer and 0.8 U Taq DNA Polymerase. The PCR reaction was carried out following the program: 95° C., 3 min; 25 cycles of (95° C., 30 s; 60° C., 30 s; 68° C., 45 s); 68° C., 5 min. PCR products were verified by electrophoresis with a 2% agarose gel supplemented with ethidium bromide. In the 2^{nd} round PCR, the PCR product of 1^{st} round PCR was barcoded with Unique Illumina Barcoding primers. 1 ul of PCR product from 1^{st} round PCR reaction, was added into 20 ul of 2^{nd} round PCR reaction containing 1× Standard Taq reaction buffer, 800 uM dNTP mix (200 uM each), 0.5 uM Illumina P7 and P5 index primers and 0.8 U Taq DNA Polymerase. The PCR reactions follow the program: 95° C., 3 min; 8 cycles of (95° C., 30 s; 60° C., 30 s; 68° C., 45 s); 68° C., 5 min. PCR products were verified by electrophoresis with a 2% agarose gel before pooling and gel purified using QIAquick Gel Extraction Kit (Qiagen). The DNA was quantified by the KAPA Library Quantification Kit-Illumina (KAPA Biosystems) before being subjected to next-generation sequencing on an Illumina MiSeq Instrument.

### 11. Overexpression and Purification of Recombinant TadA8r Protein.

TadA8r fused to an N-terminal hexahistidine-tagged maltose binding protein (6×His-MBP) were cloned into a pET28a vector with a TEV protease cleavage site (ENLYFQIG) installed between MBP and TadA8r.

BL21 Rosetta 2 (DE3) competent cells were transformed with the recombinant plasmids and grown on Luria broth (LB) agar plates supplemented with 50 μg/mL kanamycin and 25 μg/mL chloramphenicol. Successfully transformed bacteria were always cultured in the presence of 50 μg/mL kanamycin and 25 μg/mL chloramphenicol unless otherwise noted. Single colonies were inoculated into fresh LB medium and grown in an incubator shaker (37° C., 220 rpm) for 12-18 h. A 10 mL saturated start culture was used to inoculate 1 L fresh medium. Bacteria were grown at 37° C. until OD₆₀₀ reached 0.5. The culture was cooled down immediately to 4° C. and induced with 0.1 mM isopropyl β-d-1-thiogalactopyranoside (IPTG). Bacteria were cultured at 16° C. for an additional 20 h before pelleting by centrifugation at 4,000 g.

Bacterial pellets were lysed by sonication in buffer A (50 mM Tris, 500 mM NaCl, 10 mM β-mercaptoethanol, and 10% (v/v) glycerol; pH 7.5). Lysed bacteria were clarified by centrifugation at 4° C., 23,000 g. The supernatant was loaded onto a Ni-NTA Superflow Cartridge (Qiagen, 30761), washed with 30 mL of buffer A supplemented with 50 mM imidazole, and eluted with a gradient of imidazole from 50 mM to 500 mM in buffer A. The eluted protein was incubated with TEV protease and dialyzed in buffer A at 4° C. overnight. The protein mixture was diluted with buffer B (50 mM Tris, 50 mM NaCl, 10 mM $-mercaptoethanol, and 10% (v/v) glycerol; pH 7.0) in a volume that is two-fold to protein mixture. The diluted protein mixture was loaded onto a S column, washed with buffer C (50 mM Tris, 200 mM NaCl, 10 mM β-mercaptoethanol, and 10% (v/v) glycerol; pH 7.0), and eluted with a gradient of buffer C from 200 mM NaCl to 1M NaCl. Finally, MBP-free TadA8.20 was purified by size-exclusion chromatography (Enrich™ SEC 650 10×300 mm Column, Bio-Rad, 7801650) and concentrated to approximately 4 mg/mL. The column was balanced and eluted with buffer D (50 mM Tris, 200 mM NaCl, 10 mM $-mercaptoethanol, and 10% (v/v) glycerol; pH 7.5).

### D. Tables

In the tables below, N=G, A. T. C; W=A. T; R=A, G; Y=C, T; M=A, C; K=G, T; S=C, G.

TABLE 1
Genotypes of ABE-RAs identified in this work. Residue position
in the evolved E. coli TadA portion of ABE are indicated.

| Editor | 23 | 27 | 36 | 47 | 48 | 51 | 76 | 82 | 84 | 106 | 108 | 109 | 110 | 111 | 114 | 119 | 122 |
|---|---|---|---|---|---|---|---|---|---|---|---|---|---|---|---|---|---|
| WTTadA | W | E | H | R | P | R | I | V | L | A | D | A | K | T | A | D | H |
| ABE7.10 | R | | L | | A | L | | | F | V | N | | | | | | |
| ABE8.20 | R | | L | | A | L | Y | S | F | V | N | | | | | | |
| ABE8e | R | | L | | A | L | | | F | V | N | S | | R | | N | N |
| ABE-RA1.0 | | | | | | | | | | | G | | | | | | |
| ABE-RA1.1 | | | | | | | | | | | G | | | | | | |
| ABE-RA2.0 | | | | | A | H | F | | | | G | | R | | | | R |
| ABE-RA2.1 | | | | | A | H | | | | | G | | R | | | | R |
| ABE-RA3.0 | | D | | | A | H | F | | | | G | | R | | | | R |
| ABE-RA3.1 | | D | | K | A | H | F | | | | G | | R | | | | R |
| ABE-RA3.2 | | D | | | A | H | | | | | G | | R | | V | | R |
| ABE-RA3.3 | | D | | K | A | H | F | | | | G | | R | | V | | R |
| ABE-RA4.0 | | | | | A | H | F | | | V | G | | R | H | | N | |
| ABE-RA4.1 | | | | | A | H | F | | | V | G | | R | H | | N | R |
| ABE-RA4.2 | | | | | A | H | F | | | V | G | | R | H | | N | |
| ABE-RA4.3 | | | | | A | H | F | | | V | G | | R | H | | N | |
| ABE-RA4.4 | | | | | A | H | F | | | V | G | | R | H | | N | |
| ABE-RA4.5 | | | | | A | H | F | | | V | G | | R | H | | N | |
| ABE-RA4.6 | | | | | A | H | F | | | V | G | | R | H | | N | |
| ABE-RA5.0 | R | | L | K | A | L | F | S | | V | G | | R | H | | N | |
| ABE-RA5.1 | R | | L | K | A | L | F | S | | V | G | | R | H | | N | N |
| ABE-RA5.2 | R | | L | K | A | L | Y | S | | V | G | | R | H | V | N | |
| ABE-RA5.3 | R | | L | K | A | L | F | S | | V | G | S | R | H | V | N | |
| ABE-RA5.4 | R | | | K | A | L | Y | S | | V | G | S | R | H | V | N | |
| ABE-RA5.5 | R | | L | K | A | L | Y | S | | V | G | | R | H | V | N | |
| ABE-RA5.6 | R | | L | K | A | L | Y | S | | V | G | | R | H | V | N | |

| Editor | 123 | 126 | 127 | 146 | 147 | 149 | 152 | 154 | 155 | 156 | 157 | 161 | 166 | 167 |
|---|---|---|---|---|---|---|---|---|---|---|---|---|---|---|
| WTTadA | H | M | N | S | D | F | R | Q | E | I | K | K | T | D |
| ABE7.10 | Y | | | C | Y | | P | | V | F | N | | | |
| ABE8.20 | | | | C | R | | P | R | V | F | N | | | |
| ABE8e | Y | | | C | | Y | P | | V | F | N | | I | N |
| ABE-RA1.0 | | | | | | | | | | | | | | |
| ABE-RA1.1 | | | | | | | | | | | | N | | |
| ABE-RA2.0 | | I | K | | | | | | | | | N | | |
| ABE-RA2.1 | | I | K | | | | | | | | | | | |
| ABE-RA3.0 | | I | K | | | | | | | | | N | | |
| ABE-RA3.1 | | I | K | | | | | | | | | N | | |
| ABE-RA3.2 | | I | K | | | | | | | | | | | |
| ABE-RA3.3 | | I | K | | | | | | | | | | | |
| ABE-RA4.0 | Y | I | K | | R | | P | R | V | F | | | | |
| ABE-RA4.1 | Y | I | K | | R | | P | R | V | F | | | | |
| ABE-RA4.2 | Y | I | K | C | R | | P | R | V | F | | | | |
| ABE-RA4.3 | Y | I | K | | R | | P | R | V | F | N | | | |
| ABE-RA4.4 | Y | I | K | | R | | P | R | V | F | | N | | |
| ABE-RA4.5 | Y | I | K | | R | | P | R | V | F | | | I | |
| ABE-RA4.6 | Y | I | K | | R | | P | R | V | F | | | | N |
| ABE-RA5.0 | Y | I | K | C | R | | P | R | V | F | | | | |
| ABE-RA5.1 | Y | I | K | C | R | | P | R | V | F | | | | |
| ABE-RA5.2 | Y | I | K | C | R | | P | R | V | F | | | | |
| ABE-RA5.3 | Y | I | K | | R | | P | R | V | F | | | | |
| ABE-RA5.4 | Y | I | K | | R | | P | R | V | F | | | | |
| ABE-RA5.5 | Y | I | K | C | R | | P | R | V | F | N | N | I | N |
| ABE-RA5.6 | Y | I | K | C | R | | P | R | V | F | | | I | N |

Supplementary Table 1.
Primers used for generating sgRNA plasmids

| | | | | SEQ |
|---|---|---|---|---|
| | targeting | | | ID |
| plasmid | site | Primer | sequence | NO: |
| | site 1-23 | Fwd | agagcUagaaatagcaagttaaaataagg | 34 |
| | | primer | | |
| 034c | site 1 | Rev | agctcUaaaacGCAGTCTATGCTTTGTGTTCggtgtttcgtcctt | 35 |
| | | primer | tccacaag | |
| 034d | site 2 | Rev | agctcUaaaacCCACCCAAGTGATCACACTTCggtgtttcgtc | 36 |
| | | primer | ctttccacaag | |
| 060e | site 3 | Rev | agctcUaaaacccccaaaggtgaccgtcctgcggtgtttcgtcctttccacaag | 37 |
| | | primer | | |
| 122e | site 4 | Rev | agctcUaaaacCCAAGACAAACTTGCATCCTCggtgtttcgtc | 38 |
| | | primer | ctttccacaag | |
| 060b | site 5 | Rev | agctcUaaaaccctgacaatcgataggtaccggtgtttcgtcctttccacaag | 39 |
| | | primer | | |
| 034j | site 6 | Rev | agctcUaaaacGCAGTCTATGCCTCATACTCggtgtttcgtcct | 40 |
| | | primer | ttccacaag | |
| 034n | site 7 | Rev | agctcUaaaacGCCCTGGCCTGGGTCAATCCggtgtttcgtcct | 41 |
| | | primer | ttccacaag | |
| 034r | site 8 | Rev | agctcUaaaacGCAGTCTATCCTTGGTCTTCggtgtttcgtcctt | 42 |
| | | primer | tccacaag | |
| 034v | site 9 | Rev | agctcUaaaacCAAAGGTGACCGTCCTGGCTCggtgtttcgt | 43 |
| | | primer | cctttccacaag | |
| 034w | site 10 | Rev | agctcUaaaacCCCAAGTGATCACACTTGTCggtgtttcgtcct | 44 |
| | | primer | ttccacaag | |
| 034x | site 11 | Rev | agctcUaaaacTGGCCTGGGTCAATCCTTGGCggtgtttcgtc | 45 |
| | | primer | ctttccacaag | |
| 122b | site 12 | Rev | agctcUaaaaccagctacctgaagtacttggCggtgtttcgtcctttccacaag | 46 |
| | | primer | | |
| 034m | site 13 | Rev | agctcUaaaacTGACTCATCATTATCTCATCggtgtttcgtcctt | 47 |
| | | primer | tccacaag | |
| 120d | site 14 | Rev | agctcUaaaactttaatcataacaattgcttCggtgtttcgtcctttccacaag | 48 |
| | | primer | | |
| 120n | site 15 | Rev | agctcUaaaaccatttcttttggaatgtattcggtgtttcgtcctttccacaag | 49 |
| | | primer | | |
| 1200 | site 16 | Rev | agctcUaaaacatttcttttggaatgtattcggtgtttcgtcctttccacaag | 50 |
| | | primer | | |
| 120p | site 17 | Rev | agctcUaaaactttcttttggaatgtattcaCggtgtttcgtcctttccacaag | 51 |
| | | primer | | |
| 121f | site 18 | Rev | agctcUaaaaccactatctcaatgcaaatatCggtgtttcgtcctttccacaag | 52 |
| | | primer | | |
| 121g | site 19 | Rev | agctcUaaaacgcaccttggcgcagcggtggCggtgtttcgtcctttccacaag | 53 |
| | | primer | | |
| 121j | site 20 | Rev | agctcUaaaacgcttgcccccttgggccttaCggtgtttcgtcctttccacaag | 54 |
| | | primer | | |
| 121k | site 21 | Rev | agctcUaaaaccgcaggccacggtcacctgcggtgtttcgtcctttccacaag | 55 |
| | | primer | | |
| 034z | site 22 | Rev | agctcUaaaacTCACGTGCTCAGTCTGGGCCggtgtttcgtcct | 56 |
| | | primer | ttccacaag | |
| 034y | site 23 | Rev | agctcUaaaacTTCTTCTTCTGCTCGGACTCggtgtttcgtcctt | 57 |
| | | primer | tccacaag | |
| | site R | Fwd | agtactcUggaaacagaatctactaaaacaaggc | 58 |
| | loop 1-6 | primer | | |
| 069a | R loop 1 | Rev | agagtacUaaaacTAGGACACATGCTGTCTACCACggtgttt | 59 |
| | | primer | cgtcctttccacaag | |
| 069b | R loop 2 | Rev | agagtacUaaaacCCCCAAAGGCCAGGCTGTAAATCggtg | 60 |
| | | primer | tttcgtcctttccacaag | |
| 069c | R loop 3 | Rev | agagtacUaaaacTGTTTAGCACATTACCTGACACggtgttt | 61 |
| | | primer | cgtcctttccacaag | |
| 069d | R loop 4 | Rev | agagtacUaaaacACCCCATGCACCCTCCTCCACCggtgttt | 62 |
| | | primer | cgtcctttccacaag | |
| 069f | R loop 5 | Rev | agagtacUaaaactggctcaatcaatcctcttgccggtgtttcgtcctttccaca | 63 |
| | | primer | ag | |
| 069k | R loop 6 | Rev | agagtacUaaaacttatgatacttcgcacactagtCggtgtttcgtcctttcca | 64 |
| | | primer | caag | |
| | site 24-33 | Fwd | agagcUagaaatagcaagttaaaataagg | 34 |
| | | primer | | |
| 119a | site 24 | Rev | agctcUaaaacGGAGTTTGGCCTTGTTAACCggtgtttcgtcct | 65 |
| | | primer | ttccacaag | |
| 119b | site 25 | Rev | agctcUaaaacCTAATCCCGGAACTGGACCCggtgtttcgtcc | 66 |
| | | primer | tttccacaag | |
| 119k | site 26 | Rev | agctcUaaaacagcccagcagtctatccttgCggtgtttcgtcctttccacaag | 67 |
| | | primer | | |
| 119f | site 27 | Rev | agctcUaaaacGCCGTTTGTACTTTGTCCTCggtgtttcgtcctt | 68 |
| | | primer | tccacaag | |
| 119d | site 28 | Rev | agctcUaaaacGCCAGATAATACGGGTCATCggtgtttcgtcc | 69 |
| | | primer | tttccacaag | |
| 119i | site 29 | Rev | agctcUaaaacAGTCATGGTTTGATGTCTCCggtgtttcgtcct | 70 |
| | | primer | ttccacaag | |
| 128a | site 30 | Rev | agctcUaaaacGTGACAAGTGTGATCACTTGCggtgtttcgtc | 71 |
| | | primer | ctttccacaag | |
| 128b | site 31 | Rev | agctcUaaaacTGATGTCTCCTGCAGTCTATCggtgtttcgtc | 72 |
| | | primer | ctttccacaag | |
| 129a | site 32 | Rev | agctcUaaaacCTTCTTCATCTGCAAGTCATCggtgtttcgtc | 73 |
| | | primer | ctttccacaag | |
| 129d | site 33 | Rev | agctcUaaaactggaaaaatggctttgaatcggtgtttcgtcctttccacaag | 74 |
| | | primer | | |
| | site 34-43 | Fwd | agtactcUggaaacagaatctactaaaacaaggc | 58 |
| | | primer | | |
| 069a | site 34 | Rev | agagtacUaaaacTAGGACACATGCTGTCTACCACggtgttt | 59 |
| | | primer | cgtcctttccacaag | |
| 069b | site 35 | Rev | agagtacUaaaacCCCCAAAGGCCAGGCTGTAAATCggtg | 60 |
| | | primer | tttcgtcctttccacaag | |
| 069c | site 36 | Rev | agagtacUaaaacTGTTTAGCACATTACCTGACACggtgttt | 61 |
| | | primer | cgtcctttccacaag | |
| 069d | site 37 | Rev | agagtacUaaaacACCCCATGCACCCTCCTCCACCggtgttt | 62 |
| | | primer | cgtcctttccacaag | |
| 069k | site 38 | Rev | agagtacUaaaacttatgatacttcgcacactagtCggtgtttcgtcctttccac | 64 |
| | | primer | aag | |
| 069l | site 39 | Rev | agagtacUaaaacgtcaggcctctgtccctctgtaCggtgtttcgtcctttccac | 75 |
| | | primer | aag | |
| 115h | site 40 | Rev | agagtacUaaaacAGGCTGTTGTCATACTTCTCATCggtgtt | 76 |
| | | primer | tcgtcctttccacaag | |
| 115i | site 41 | Rev | agagtacUaaaacGGTAATGACTAAGATGACTGCCggtgtt | 77 |
| | | primer | tcgtcctttccacaag | |
| 115k | site 42 | Rev | agagtacUaaaacGGGTACAATCCTACTCTAGTCCggtgttt | 78 |
| | | primer | cgtcctttccacaag | |
| 115m | site 43 | Rev | agagtacUaaaacTGCTGTCACAGTTAGCTCAGCCggtgttt | 79 |
| | | primer | cgtcctttccacaag | |
| | site 44- | Rev | ATCTacacUtagtagaaattcggtgtttcgtcctttccacaag | 80 |
| | 49_LbABE | primer | | |
| 113a | site | Fwd | agtgtAGAUTGCTGCAAGTAAGCATGCATTTGtttttttaa | 81 |
| | 44_LbABE | primer | gcttgggccgctcgag | |
| 113b | site | Fwd | agtgtAGAUCTAGACAGGGGCTAGTATGTGCAtttttttaa | 82 |
| | 45_LbABE | primer | gcttgggccgctcgag | |
| 113c | site | Fwd | agtgtAGAUCAGCTATTCAGGCTGGCCCGCCCtttttttaa | 83 |
| | 46_LbABE | primer | gcttgggccgctcgag | |
| 113d | site | Fwd | agtgtAGAUGAAGCACATCAAGGACATTCTAAtttttttaa | 84 |
| | 47_LbABE | primer | gcttgggccgctcgag | |
| 113e | site | Fwd | agtgtAGAUGGATAAGCACAGTTTTAAATAGTtttttttaa | 85 |
| | 48_LbABE | primer | gcttgggccgctcgag | |
| 113f | site | Fwd | agtgtAGAUGTTTAAACACACCGGGTTAATAAtttttttaa | 86 |
| | 49_LbABE | primer | gcttgggccgctcgag | |
| | site 44- | Rev | acaagagUagaaattcggtgtttcgtcctttccacaag | 87 |
| | 49_enAsABE | primer | | |
| 114a | site | Fwd | actcttgUAGATTGCTGCAAGTAAGCATGCATTTGtttttt | 88 |
| | 44_enAsABE | primer | taagcttgggccgctcgag | |
| 114b | site | Fwd | actcttgUAGATCTAGACAGGGGCTAGTATGTGCAttttt | 89 |
| | 45_enAsABE | primer | ttaagcttgggccgctcgag | |
| 114c | site | Fwd | actcttgUAGATCAGCTATTCAGGCTGGCCCGCCCtttttt | 90 |
| | 46_enAsABE | primer | taagcttgggccgctcgag | |
| 114d | site | Fwd | actcttgUAGATGAAGCACATCAAGGACATTCTAAttttt | 91 |
| | 47_enAsABE | primer | ttaagcttgggccgctcgag | |
| 114e | site | Fwd | actcttgUAGATGGATAAGCACAGTTTTAAATAGTtttttt | 92 |
| | 48_enAsABE | primer | taagcttgggccgctcgag | |
| 114f | site | Fwd | actcttgUAGATGTTTAAACACACCGGGTTAATAAtttttt | 93 |
| | 49_enAsABE | primer | taagcttgggccgctcgag | |
| | site 50-53 | Fwd | agagcUagaaatagcaagttaaaataagg | 34 |
| | | primer | | |
| PCSK9 | site | Rev | agctcUaaaacgcttgcccccttgggccttaCggtgtttcgtcctttccacaag | 54 |
| | 50_PCSK9 | primer | | |
| PCSK9 | site | Rev | agctcUaaaaccgcaggccacggtcacctgcggtgtttcgtcctttccacaag | 55 |
| | 51_PCSK9 | primer | | |
| ABCA4 | site | Rev | agctcUaaaacctccagggcgaactTcgacaCggtgtttcgtcctttccacaag | 94 |
| | 52_ABCA4 | primer | | |
| ABCA4 | site | Rev | agctcUaaaaccctctccagggcgaactTcgCggtgtttcgtcctttccacaag | 95 |
| | 53_ABCA4 | primer | | |

Supplementary Table 2.
DNA templates used for error prone PCR and guide RNA protospacer information for
each round of selection

| | | TadA | Guide RNA | Guide RNA | |
|---|---|---|---|---|---|
| Round | Template | mutations | protospacer 1 | protospacer 2 | Guide RNA protospacer 3 |
| 1 | wildtype | wildtype | GctctgATCtg | / | 1 |
| | TadA | | aataccacg | | |
| | | | (SEQ ID | | |
| | | | NO: 96) | | |
| 2 | ABE- | D108G, K161N | GCTTGatcG | GactgATCGcaacag | / |
| | RA1.0 | | GAGAGGC | acaat (SEQ ID | |
| | and | | TATT (SEQ | NO: 99) | |
| | ABE- | | ID NO: 97) | | |
| | RA1.1 | | | | |
| 3 | ABE_ | P48A, R51H, | GCTTGatcG | GactgATCGcaacag | / |
| | RA2.0, | I76F, D108G, | GAGAGGC | acaat (SEQ ID | |
| | ABE- | K110R, M126I, | TATT (SEQ | NO: 99) | |
| | RA2.1 | N127K, H122R, | ID NO: 97) | | |
| | and | K161N | | | |
| | ABE- | | | | |
| | RA2.2 | | | | |
| 4 | / | part of the | GCTTGatcG | GactgATCGcaacag | / |
| | | mutations | GAGAGGC | acaat (SEQ ID | |
| | | accumulated | TATT (SEQ | NO: 99) | |
| | | and mutations | ID NO: 97) | | |
| | | from TadA7.10, | | | |
| | | TadA8.20, | | | |
| | | TadA8e | | | |
| 5 | / | part of the | TtctttTcAGtg | gTCAggcTGCaatgt | TacggcGtAGtgCacctgGa |
| | | mutations | ccattggg | gaata (SEQ ID | (SEQ ID NO: 101) |
| | | accumulated | (SEQ ID | NO: 100) | |
| | | and mutations | NO: 98) | | |
| | | from TadA7.10, | | | |
| | | TadA8.20, | | | |
| | | TadA8e | | | |

SUPPLEMENTARY TABLE 3
Generation of DNA fragments used for overlapping PCR in DNA shuffling

| entry | Fwd primer | Rev primer | DNA template | shuffled amino acids |
|---|---|---|---|---|
| 1A | YX209 | WT1681 | plasmids containing | R51(R/H); I76(I/F); |
| | | | TadA_P48A and | with P48A fixed |
| | | | TadA_P48A_R51H (1:1) | |
| 1B | WT1679/WT1680 | WT1682 | DNA ultramer | L84(L/F); |
| | (1:1) | | WT1675/WT1676 | A106(A/V); |
| | | | (1:1) | K110(K/R); |
| | | | | T111(T/R); |
| | | | | D119(D/N); |
| | | | | H122(H/R); |
| | | | | H123(H/Y); |
| | | | | M126(M/I); |
| | | | | N127(N/K); with |
| | | | | D108G fixed |
| 1C | WT1683 | YX210 | DNA ultramer | S146(S/C); |
| | | | WT1677/WT1678 | D147(D/R); |
| | | | (1:1) | F149(F/Y); |
| | | | | R152(R/P); |
| | | | | Q154(Q/R); |
| | | | | E155(E/V); I156(I/F); |
| | | | | K157(K/N); |
| | | | | K161(K/N); |
| | | | | T166(T/I); |
| | | | | D167(D/N) |
| 2A | YX209 | YX443 | TadA8.20 | W23(W/R); |
| | | | | E27(E/D) |
| 2B | YX444 | YX445 | / | H36(H/L); R47(R/K); |
| | | | | P48(P/A); H51(H/L) |
| 2C | YX446 | YX447/YX448 | TadA8.20 | I76(F/Y); V82(V/S); |
| | | (1:1) | | L84(L/F); |
| 2D | YX458 | YX450/YX451 | / | M94(M/V); |
| | | (1:1) | | D108(G/N); |
| | | | | A109(A/S); |
| | | | | H111(H/R); |
| | | | | A114(A/V); with |
| | | | | A106V, K110R, |
| | | | | D119N fixed |
| 2E | YX452 | YX210 | plasmids containing | H122(H/N); |
| | | | TadA_S146S and | S146(S/C); with |
| | | | TadA_S146C (1:1) | H123(H/Y); |
| | | | with all other | M126(M/I); |
| | | | mutations listed | N127(N/K); |
| | | | in the table fixed | D147(D/R); |
| | | | | R152(R/P); |
| | | | | Q154(Q/R); |
| | | | | E155(E/V); I156(I/F) |
| | | | | fixed |

Supplementary Table 4.
DNA oligos used for generation of TadA* libraries and oligos used for amplify and
sequencing TadA* variants

| | | SEQ ID |
|---|---|---|
| Primer | Sequence | NO: |
| YX209 | GATTGGTCTCAacctgcaggtgcagtaaggaggaaaaaaaaatg | 102 |
| YX210 | GATTGGTCTCAgtccccggtgtttcgctaccgga | 103 |
| WT1679 | ccaccctgtatgtgacattcgagccatgcgtgatgtg | 104 |
| WT1680 | ccaccctgtatgtgacactggagccatgcgtgatgtg | 105 |
| WT1681 | tgtcacatacagggtggcatcgaWcaggcggtaattctgcatg | 106 |
| WT1682 | cgtctgccaggattccctctgtgatctccacccggtg | 107 |
| WT1683 | ggaatcctggcagacgagtgcgccgccctgctg | 108 |
| WT1675 | gagccatgcgtgatgtgcgcaggagcaatgatccacagcaggatcggaagagtggtgttcggag | 109 |
| | YgcgggGcgccaRgcgcggcgcagcaggctccctgatgRatgtgctgcRcYaccccggca | |
| | tRaaScaccgggtggagatcacag | |
| WT1676 | gagccatgcgtgatgtgcgcaggagcaatgatccacagcaggatcggaagagtggtgttcggag | 110 |
| | YgcgggGcgccaRgACCggcgcagcaggctccctgatgRatgtgctgcRcYaccccgg | |
| | catRaaScaccgggtggagatcacag | |
| WT1677 | gtgcgccgccctgctgWgccgtttctWtagaatgcSgagacRggWgWtcaaKgcccaga | 111 |
| | agaaSgcacagagctccaYcRactccggtagcgaaacaccg | |
| WT1678 | gtgcgccgccctgctgWgcGAtttctWtagaatgcSgagacRggWgWtcaaKgcccag | 112 |
| | aagaaSgcacagagctccaYcRactccggtagcgaaacaccg | |
| YX443 | ggcgcccacggggacWtctctttcatcccRtgctcgctttgc | 113 |
| YX444 | ccccgtgggcgccgtgctggtgcWcaacaatagagtgatcggagaggg | 114 |
| YX445 | gcggtagggtcgtggWggccgattgScYtgttccatccctctccgatcactct | 115 |
| YX446 | cacgaccctaccgcacacg | 116 |
| YX447 | acatcacgcatggctcgaRtgtcacatacagggtggcatcgWacaggcggtaattctgca | 117 |
| YX448 | acatcacgcatggctcgaRtgtcgaatacagggtggcatcgWacaggcggtaattctgca | 118 |
| YX458 | gccatgcgtgatgtgcgcaggagcaRtgatccacagcaggatcggaagagtggtgttcgg | 119 |
| YX450 | catTcatcagggagcctRctgcgccgYGcCtggMgCcccgCActccgaacaccactcttc | 120 |
| YX451 | catTcatcagggagcctRctgcgccgYGcCtggMgTtccgCActccgaacaccactcttc | 121 |
| YX452 | ggctccctgatgAatgtgctgMacTaccccggc | 122 |
| WT022 | CATTTTGCGCTTCAGCCAT | 123 |
| YX140 | cagtgatcaccgcccatcc | 124 |

Supplementary Table 5.
Antibiotic selection plasmids and their corresponding E. coli antibiotic minimum
inhibitory concentrations (MICs).

| | | | | | | MIC in | Selection |
|---|---|---|---|---|---|---|---|
| | | | SEQ | In- | Position | S1030 | antibiotic |
| | Antibiotic | | ID | activating | of A in | cells | concentration |
| Round | resistance | Target sequence | NO: | mutation | protospacer | (ug/ml) | (ug/ml) |
| 1 | Cam^{R} | gctctgATCtgaata | 96 | W106* | 7 | 8 | 8, 16, 32, 64 |
| | | ccacg | | | | | |
| 2 | Kan^{R} | GCTTGatcGGA | 97 | W15*- | 6, 6 | 4 | 12.5, 25, 50 |
| | | GAGGCTATT | | W24* | | | |
| | | gactgATCGcaac | 99 | | | | |
| | | agacaat | | | | | |
| 3 | Kan^{R} | GCTTGatcGGA | 97 | W15*- | 6,6 | 4 | 50, 100, 200 |
| | | GAGGCTATT | | W24* | | | |
| | | gactgATCGcaac | 99 | | | | |
| | | agacaat | | | | | |
| 4 | Kan^{R} | GCTTGatcGGA | 97 | W15*- | 6, 6 | 4 | 100, 200, |
| | | GAGGCTATT | | W24* | | | 400 |
| | | gactgATCGcaac | 99 | | | | |
| | | agacaat | | | | | |
| 5 | Cam^{R} | ttctttTcAGtgccatt | 98 | R18*- | 6, 6 | 1 | 16, 32, 64, |
| | | ggg | | R65*- | | | 128 |
| | | gTCAggcTGCaa | 100 | H193Y | | | |
| | | tgtgaata | | | | | |
| | | TacggcGtAGtgC | 101 | | | | |
| | | acctgGa | | | | | |

Supplementary table 6.
Sequence of DNA or RNA used in in vitro DNA deamination assays

| | | SEQ |
|---|---|---|
| Oligo | Sequence | ID NO |
| E. coli tRNA | GCAUCCGUAGCUCAGCUGGAUAGAGUACUCGGCUAC | 125 |
| | GAACCGAGCGGUCGGAGGUUCGAAUCCUCCCGGAUG | |
| | CACCA | |
| reverse transcription | TCCGAATAGCGCCCTTCCCCTTGCCCGGCGTTAATGAT | 126 |
| primer | TTGCCCAAATGGTGCATCCG | |
| Fwd primer for RT- | ACACTCTTTCCCTACACGACGCTCTTCCGATCTNNNNG | 127 |
| PCR | CATCCGTAGCTCAGCTGG | |
| Rev primer for RT- | GACTGGAGTTCAGACGTGTGCTCTTCCGATCTTCCGAA | 128 |
| PCR | TAGCGCCCTTCC | |
| GA probe | /56-FAM/TGGGTTGGTGATCGTTTGGTGG | 129 |
| TA probe | /56-FAM/TGGGTTGGTTATCGTTTGGTGG | 130 |

Suppleme + B3: E113ntary Table 7.
Illumina primers used for next generation sequencing

| | | | SEQ |
|---|---|---|---|
| | | | ID |
| | | Sequence | NO |
| site 1_Fwd | YX220 | ACACTCTTTCCCTACACGACGCTCTTCCGATCTNNNNCCA | 131 |
| | | GCCCCATCTGTCAAACT | |
| site 1_Rev | YX221 | TGGAGTTCAGACGTGTGCTCTTCCGATCTTGAATGGATTC | 132 |
| | | CTTGGAAACAATGA | |
| site 2_Fwd | YX473 | ACACTCTTTCCCTACACGACGCTCTTCCGATCTNNNNAAT | 133 |
| | | GTGTCAACTCTTGACAGGGC | |
| site 2_Rev | YX474 | GACTGGAGTTCAGACGTGTGCTCTTCCGATCTGAGCTGC | 134 |
| | | AGGTGTAATGAAGACC | |
| site 3_Fwd | YX473 | ACACTCTTTCCCTACACGACGCTCTTCCGATCTNNNNAAT | 133 |
| | | GTGTCAACTCTTGACAGGGC | |
| site 3_Rev | YX474 | GACTGGAGTTCAGACGTGTGCTCTTCCGATCTGAGCTGC | 134 |
| | | AGGTGTAATGAAGACC | |
| site 4_Fwd | YX327 | ACACTCTTTCCCTACACGACGCTCTTCCGATCTNNNNCCG | 135 |
| | | ACAGCCAGTGGTTAAGT | |
| site 4_Rev | YX328 | TGGAGTTCAGACGTGTGCTCTTCCGATCTGCTTTTCACCG | 136 |
| | | ACTGCACAG | |
| site 5_Fwd | YX473 | ACACTCTTTCCCTACACGACGCTCTTCCGATCTNNNNAAT | 133 |
| | | GTGTCAACTCTTGACAGGGC | |
| site 5_Rev | YX474 | GACTGGAGTTCAGACGTGTGCTCTTCCGATCTGAGCTGC | 134 |
| | | AGGTGTAATGAAGACC | |
| site 6_Fwd | YX325 | ACACTCTTTCCCTACACGACGCTCTTCCGATCTNNNNAGA | 137 |
| | | GACTGATTGCGTGGAGT | |
| site 6_Rev | YX326 | TGGAGTTCAGACGTGTGCTCTTCCGATCTCACTCCAGCCT | 138 |
| | | AGGCAACAA | |
| site 7_Fwd | YX939 | ACACTCTTTCCCTACACGACGCTCTTCCGATCTNNNNAGC | 139 |
| | | ATGCATTTGTAGGCTTGATG | |
| site 7_Rev | YX334 | TGGAGTTCAGACGTGTGCTCTTCCGATCTCCCAGCCAAAC | 140 |
| | | TTGTCAACC | |
| site 8_Fwd | YX516 | ACACTCTTTCCCTACACGACGCTCTTCCGATCTNNNNTTG | 141 |
| | | CTTATTGCTGAGGGGCA | |
| site 8_Rev | YX517 | TGGAGTTCAGACGTGTGCTCTTCCGATCTACCTCTCTCCT | 142 |
| | | CCAGCTGAG | |
| site 9_Fwd | YX473 | ACACTCTTTCCCTACACGACGCTCTTCCGATCTNNNNAAT | 133 |
| | | GTGTCAACTCTTGACAGGGC | |
| site 9_Rev | YX474 | GACTGGAGTTCAGACGTGTGCTCTTCCGATCTGAGCTGC | 134 |
| | | AGGTGTAATGAAGACC | |
| site 10_Fwd | YX473 | ACACTCTTTCCCTACACGACGCTCTTCCGATCTNNNNAAT | 133 |
| | | GTGTCAACTCTTGACAGGGC | |
| site 10_Rev | YX474 | GACTGGAGTTCAGACGTGTGCTCTTCCGATCTGAGCTGC | 134 |
| | | AGGTGTAATGAAGACC | |
| site 11_Fwd | YX939 | ACACTCTTTCCCTACACGACGCTCTTCCGATCTNNNNAGC | 139 |
| | | ATGCATTTGTAGGCTTGATG | |
| site 11_Rev | YX334 | TGGAGTTCAGACGTGTGCTCTTCCGATCTCCCAGCCAAAC | 140 |
| | | TTGTCAACC | |
| site 12_Fwd | YX829 | ACACTCTTTCCCTACACGACGCTCTTCCGATCTNNNNggctt | 143 |
| | | atgaaggcagagactgag | |
| site 12_Rev | YX830 | TGGAGTTCAGACGTGTGCTCTTCCGATCTgttacctctcctttccaag | 144 |
| | | gcac | |
| site 13_Fwd | YX331 | ACACTCTTTCCCTACACGACGCTCTTCCGATCTNNNNGTC | 145 |
| | | TGAGGTCACACAGTGGG | |
| site 13_Rev | YX332 | TGGAGTTCAGACGTGTGCTCTTCCGATCTCTGAGAGCAG | 146 |
| | | GGACCACATC | |
| site 14_Fwd | YX766 | ACACTCTTTCCCTACACGACGCTCTTCCGATCTNNNNtacac | 147 |
| | | ccaattcttcactgatgc | |
| site 14_Rev | YX767 | GACTGGAGTTCAGACGTGTGCTCTTCCGATCTcaaacaaacgtta | 148 |
| | | tgacaaacctcc | |
| site 15_Fwd | YX775 | ACACTCTTTCCCTACACGACGCTCTTCCGATCTNNNNggtga | 149 |
| | | ttcaaagggtatcaggcc | |
| site 15_Rev | YX776 | GACTGGAGTTCAGACGTGTGCTCTTCCGATCTggcactcataaa | 150 |
| | | cagaaggttctacc | |
| site 16_Fwd | YX775 | ACACTCTTTCCCTACACGACGCTCTTCCGATCTNNNNggtga | 149 |
| | | ttcaaagggtatcaggcc | |
| site 16_Rev | YX776 | GACTGGAGTTCAGACGTGTGCTCTTCCGATCTggcactcataaa | 150 |
| | | cagaaggttctacc | |
| site 17_Fwd | YX775 | ACACTCTTTCCCTACACGACGCTCTTCCGATCTNNNNggtga | 149 |
| | | ttcaaagggtatcaggcc | |
| site 17_Rev | YX776 | GACTGGAGTTCAGACGTGTGCTCTTCCGATCTggcactcataaa | 150 |
| | | cagaaggttctacc | |
| site 18_Fwd | YX797 | ACACTCTTTCCCTACACGACGCTCTTCCGATCTNNNNcctgg | 151 |
| | | cctcactggatactc | |
| site 18_Rev | YX940 | TGGAGTTCAGACGTGTGCTCTTCCGATCTgaatgactgaatcggaa | 152 |
| | | caaggc | |
| site 19_Fwd | YX799 | ACACTCTTTCCCTACACGACGCTCTTCCGATCTNNNNctagc | 153 |
| | | cttgcgttccgagg | |
| site 19_Rev | YX800 | TGGAGTTCAGACGTGTGCTCTTCCGATCTcctgcagtccccaagatc | 154 |
| | | g | |
| site 20_Fwd | YX803 | ACACTCTTTCCCTACACGACGCTCTTCCGATCTNNNNagggt | 155 |
| | | gcttgagttgatcctg | |
| site 20_Rev | YX804 | TGGAGTTCAGACGTGTGCTCTTCCGATCTatgctggcctcagctggt | 156 |
| | | g | |
| site 21_Fwd | YX805 | ACACTCTTTCCCTACACGACGCTCTTCCGATCTNNNNcctca | 157 |
| | | cagaaggatgtcggag | |
| site 21_Rev | YX806 | TGGAGTTCAGACGTGTGCTCTTCCGATCTtgcctgtagtgctgacgt | 158 |
| | | c | |
| site 22_Fwd | YX942 | ACACTCTTTCCCTACACGACGCTCTTCCGATCTNNNNtgctg | 159 |
| | | caagtaagcatgcatttg | |
| site 22_Rev | YX629 | TGGAGTTCAGACGTGTGCTCTTCCGATCTCCCAGCCAAAC | 140 |
| | | TTGTCAACC | |
| site 23_Fwd | YX561 | ACACTCTTTCCCTACACGACGCTCTTCCGATCTNNNNCAG | 160 |
| | | CTCAGCCTGAGTGTTGA | |
| site 23_Rev | YX941 | TGGAGTTCAGACGTGTGCTCTTCCGATCTctgcttcgtggcaatgcg | 161 |
| R loop | YX743 | ACACTCTTTCCCTACACGACGCTCTTCCGATCTNNNNcctgc | 162 |
| 1_Fwd | | agtctcctgcttctctg | |
| R loop | YX744 | GACTGGAGTTCAGACGTGTGCTCTTCCGATCTaacccagatgag | 163 |
| 1_Rev | | aggatgaaggc | |
| R loop | YX587 | ACACTCTTTCCCTACACGACGCTCTTCCGATCTNNNNGGA | 164 |
| 2_Fwd | | CATTTCCACCGCAAAATG | |
| R loop | YX588 | TGGAGTTCAGACGTGTGCTCTTCCGATGCTACAGAAAGG | 165 |
| 2_Rev | | TCAGCAGC | |
| R loop | YX745 | ACACTCTTTCCCTACACGACGCTCTTCCGATCTNNNNgctgt | 166 |
| 3_Fwd | | ggcatccagagacatgg | |
| R loop | YX945 | TGGAGTTCAGACGTGTGCTCTTCCGATCTctctttgctccagatttccc | 167 |
| 3_Rev | | ttc | |
| R loop | YX946 | ACACTCTTTCCCTACACGACGCTCTTCCGATCTNNNNgaatc | 168 |
| 4_Fwd | | ctggacaaggtttgaagg | |
| R loop | YX592 | TGGAGTTCAGACGTGTGCTCTTCCGATCTTCCTGAGGTCT | 169 |
| 4_Rev | | AGGAACCCG | |
| R loop | YX835 | ACACTCTTTCCCTACACGACGCTCTTCCGATCTNNNNcatga | 170 |
| 5_Fwd | | aactgtagccccagctac | |
| R loop | YX836 | TGGAGTTCAGACGTGTGCTCTTCCGATCTacttggaaccaacccaa | 171 |
| 5_Rev | | atattcctc | |
| R loop | YX845 | ACACTCTTTCCCTACACGACGCTCTTCCGATCTNNNNcactg | 172 |
| 6_Fwd | | gcctttattcagtccctc | |
| R loop | YX846 | TGGAGTTCAGACGTGTGCTCTTCCGATCTagagcactgagcataga | 173 |
| 6_Rev | | ccaag | |
| site 24_Fwd | YX701 | ACACTCTTTCCCTACACGACGCTCTTCCGATCTNNNNGCT | 174 |
| | | TTAAACATTTGTCTGTGCG | |
| site 24_Rev | YX702 | TGGAGTTCAGACGTGTGCTCTTCCGATCTGTTTTCTGTCC | 175 |
| | | CTCCCTCAGTA | |
| site 25_Fwd | YX705 | ACACTCTTTCCCTACACGACGCTCTTCCGATCTNNNNCAG | 176 |
| | | AGAGAGCAGGACGTCACA | |
| site 25_Rev | YX706 | TGGAGTTCAGACGTGTGCTCTTCCGATCTAGCACTACCTA | 177 |
| | | CGTCAGCACCT | |
| site 26_Fwd | YX516 | ACACTCTTTCCCTACACGACGCTCTTCCGATCTNNNNTTG | 141 |
| | | CTTATTGCTGAGGGGCA | |
| site 26_Rev | YX517 | TGGAGTTCAGACGTGTGCTCTTCCGATCTACCTCTCTCCT | 142 |
| | | CCAGCTGAG | |
| site 27_Fwd | YX925 | ACACTCTTTCCCTACACGACGCTCTTCCGATCTNNNNttctgc | 178 |
| | | tcggactcaggcc | |
| site 27_Rev | YX926 | TGGAGTTCAGACGTGTGCTCTTCCGATCTaaccctatgtagcctcag | 179 |
| | | tcttcc | |
| site 28_Fwd | YX709 | ACACTCTTTCCCTACACGACGCTCTTCCGATCTNNNNGAC | 180 |
| | | AGAGGGAGAGAAACAGAGC | |
| site 28_Rev | YX710 | TGGAGTTCAGACGTGTGCTCTTCCGATCTTTCTAGATGCC | 181 |
| | | GACAAAAGGAT | |
| site 29_Fwd | YX325 | ACACTCTTTCCCTACACGACGCTCTTCCGATCTNNNNAGA | 137 |
| | | GACTGATTGCGTGGAGT | |
| site 29_Rev | YX326 | TGGAGTTCAGACGTGTGCTCTTCCGATCTCACTCCAGCCT | 138 |
| | | AGGCAACAA | |
| site 30_Fwd | YX473 | ACACTCTTTCCCTACACGACGCTCTTCCGATCTNNNNAAT | 133 |
| | | GTGTCAACTCTTGACAGGGC | |
| site 30_Rev | YX474 | GACTGGAGTTCAGACGTGTGCTCTTCCGATCTGAGCTGC | 134 |
| | | AGGTGTAATGAAGACC | |
| site 31_Fwd | YX325 | ACACTCTTTCCCTACACGACGCTCTTCCGATCTNNNNAGA | 137 |
| | | GACTGATTGCGTGGAGT | |
| site 31_Rev | YX326 | TGGAGTTCAGACGTGTGCTCTTCCGATCTCACTCCAGCCT | 138 |
| | | AGGCAACAA | |
| site 32_Fwd | YX325 | ACACTCTTTCCCTACACGACGCTCTTCCGATCTNNNNAGA | 137 |
| | | GACTGATTGCGTGGAGT | |
| site 32_Rev | YX326 | TGGAGTTCAGACGTGTGCTCTTCCGATCTCACTCCAGCCT | 138 |
| | | AGGCAACAA | |
| site 33_Fwd | YX707 | ACACTCTTTCCCTACACGACGCTCTTCCGATCTNNNNCAC | 182 |
| | | TGCTGAACCAGTCAAACTC | |
| site 33_Rev | YX708 | TGGAGTTCAGACGTGTGCTCTTCCGATCTGGCATGGGGA | 183 |
| | | AATATAAACTTG | |
| site 34_Fwd | YX743 | ACACTCTTTCCCTACACGACGCTCTTCCGATCTNNNNcctgc | 162 |
| | | agtctcctgcttctctg | |
| site 34_Rev | YX744 | GACTGGAGTTCAGACGTGTGCTCTTCCGATCTaacccagatgag | 163 |
| | | aggatgaaggo | |
| site 35_Fwd | YX587 | ACACTCTTTCCCTACACGACGCTCTTCCGATCTNNNNGGA | 164 |
| | | CATTTCCACCGCAAAATG | |
| site 35_Rev | YX588 | TGGAGTTCAGACGTGTGCTCTTCCGATGCTACAGAAAGG | 165 |
| | | TCAGCAGC | |
| site 36_Fwd | YX745 | ACACTCTTTCCCTACACGACGCTCTTCCGATCTNNNNgctgt | 166 |
| | | ggcatccagagacatgg | |
| site 36_Rev | YX945 | TGGAGTTCAGACGTGTGCTCTTCCGATCTctctttgctccagatttccc | 167 |
| | | ttc | |
| site 37_Fwd | YX946 | ACACTCTTTCCCTACACGACGCTCTTCCGATCTNNNNgaatc | 168 |
| | | ctggacaaggtttgaagg | |
| site 37_Rev | YX592 | TGGAGTTCAGACGTGTGCTCTTCCGATCTTCCTGAGGTCT | 169 |
| | | AGGAACCCG | |
| site 38_Fwd | YX845 | ACACTCTTTCCCTACACGACGCTCTTCCGATCTNNNNcactg | 172 |
| | | gcctttattcagtccctc | |
| site 38_Rev | YX846 | TGGAGTTCAGACGTGTGCTCTTCCGATCTagagcactgagcataga | 173 |
| | | ccaag | |
| site 39_Fwd | YX847 | ACACTCTTTCCCTACACGACGCTCTTCCGATCTNNNNcaga | 184 |
| | | gtctagagggcagtggtg | |
| site 39_Rev | YX848 | TGGAGTTCAGACGTGTGCTCTTCCGATCTctcccacacacattgaat | 185 |
| | | ctcctg | |
| site 40_Fwd | YX715 | ACACTCTTTCCCTACACGACGCTCTTCCGATCTNNNNCTG | 186 |
| | | ACTCAGCCCTGCAAAGG | |
| site 40_Rev | YX716 | TGGAGTTCAGACGTGTGCTCTTCCGATCTCAAGTCAGGG | 187 |
| | | GAGCGTGTC | |
| site 41_Fwd | YX717 | ACACTCTTTCCCTACACGACGCTCTTCCGATCTNNNNACG | 188 |
| | | TCTCATATGCCCCTTGG | |
| site 41_Rev | YX718 | TGGAGTTCAGACGTGTGCTCTTCCGATCTACGTAGGAATT | 189 |
| | | TTGGTGGGACA | |
| site 42_Fwd | YX721 | ACACTCTTTCCCTACACGACGCTCTTCCGATCTNNNNCCC | 190 |
| | | TGTTCCTAAAGCCCACC | |
| site 42_Rev | YX722 | TGGAGTTCAGACGTGTGCTCTTCCGATCTACTGGTTCTGT | 191 |
| | | TTGTGGCCA | |
| site 43_Fwd | YX220 | ACACTCTTTCCCTACACGACGCTCTTCCGATCTNNNNCCA | 131 |
| | | GCCCCATCTGTCAAACT | |
| site 43_Rev | YX221 | TGGAGTTCAGACGTGTGCTCTTCCGATCTTGAATGGATTC | 132 |
| | | CTTGGAAACAATGA | |
| site 44_Fwd | YX951 | ACACTCTTTCCCTACACGACGCTCTTCCGATCTNNNNccag | 192 |
| | | ggaaacgcccatgc | |
| site 44_Rev | YX654 | TGGAGTTCAGACGTGTGCTCTTCCGATCTCCCAGCCAAAC | 140 |
| | | TTGTCAACC | |
| site 45_Fwd | YX951 | ACACTCTTTCCCTACACGACGCTCTTCCGATCTNNNNccag | 192 |
| | | ggaaacgcccatgc | |
| site 45_Rev | YX654 | TGGAGTTCAGACGTGTGCTCTTCCGATCTCCCAGCCAAAC | 140 |
| | | TTGTCAACC | |
| site 46_Fwd | YX220 | ACACTCTTTCCCTACACGACGCTCTTCCGATCTNNNNCCA | 131 |
| | | GCCCCATCTGTCAAACT | |
| site 46_Rev | YX221 | TGGAGTTCAGACGTGTGCTCTTCCGATCTTGAATGGATTC | 132 |
| | | CTTGGAAACAATGA | |
| site 47_Fwd | YX659 | ACACTCTTTCCCTACACGACGCTCTTCCGATCTNNNNAAA | 193 |
| | | AGGGGCAAGCTTCAGAT | |
| site 47_Rev | YX660 | TGGAGTTCAGACGTGTGCTCTTCCGATCTAGTGAGGAGA | 194 |
| | | AGGCAGGAGG | |
| site 48_Fwd | YX661 | ACACTCTTTCCCTACACGACGCTCTTCCGATCTNNNNTGT | 195 |
| | | TCTGCCCTCACAGAGGT | |
| site 48_Rev | YX662 | TGGAGTTCAGACGTGTGCTCTTCCGATCCCAAAGGACAT | 196 |
| | | ACGGGGAG | |
| site 49_Fwd | YX663 | ACACTCTTTCCCTACACGACGCTCTTCCGATCTNNNNGTG | 197 |
| | | CGTGCTTCTTACATGCC | |
| site 49_Rev | YX664 | TGGAGTTCAGACGTGTGCTCTTCCGATCCAAGTATGCCTT | 198 |
| | | AAGCAGAACAA | |
| site | YX803 | ACACTCTTTCCCTACACGACGCTCTTCCGATCTNNNNagggt | 155 |
| 50_PCSK9_ | | gcttgagttgatcctg | |
| Fwd | | | |
| site | YX804 | TGGAGTTCAGACGTGTGCTCTTCCGATCTatgctggcctcagctggt | 156 |
| 50_PCSK9_ | | g | |
| Rev | | | |
| site | YX805 | ACACTCTTTCCCTACACGACGCTCTTCCGATCTNNNNcctca | 157 |
| 51_PCSK9_ | | cagaaggatgtcggag | |
| Fwd | | | |
| site | YX806 | TGGAGTTCAGACGTGTGCTCTTCCGATCTtgcctgtagtgctgacgt | 158 |
| 51_PCSK9_ | | c | |
| Rev | | | |
| site | YX1095 | ACACTCTTTCCCTACACGACGCTCTTCCGATCTNNNNctgtct | 199 |
| 52_ABCA4_ | | cagttctcagtccgg | |
| Fwd | | | |
| site | YX1096 | GACTGGAGTTCAGACGTGTGCTCTTCCGATCTtagctctgccttat | 200 |
| 52_ABCA4_ | | ggggagg | |
| Rev | | | |
| site | YX1095 | ACACTCTTTCCCTACACGACGCTCTTCCGATCTNNNNctgtct | 199 |
| 53_ABCA4_ | | cagttctcagtccgg | |
| Fwd | | | |
| site | YX1096 | GACTGGAGTTCAGACGTGTGCTCTTCCGATCTtagctctgccttat | 200 |
| 53_ABCA4_ | | ggggagg | |
| Rev | | | |
| site | YX581 | ACACTCTTTCCCTACACGACGCTCTTCCGATCTNNNNGTG | 201 |
| 1_OT1_Fwd | | TGGAGAGTGAGTAAGCCA | |
| site | YX582 | TGGAGTTCAGACGTGTGCTCTTCCGATCTACGGTAGGAT | 202 |
| 1_OT1_Rev | | GATTTCAGGCA | |
| site | YX583 | ACACTCTTTCCCTACACGACGCTCTTCCGATCTNNNNCAC | 203 |
| 1_OT2_Fwd | | AAAGCAGTGTAGCTCAGG | |
| site | YX584 | TGGAGTTCAGACGTGTGCTCTTCCGATCTTTTTTGGTACT | 204 |
| 1_OT2_Rev | | CGAGTGTTATTCAG | |
| site | YX787 | ACACTCTTTCCCTACACGACGCTCTTCCGATCTNNNNTCC | 205 |
| 22_OT1_Fwd | | CCTGTTGACCTGGAGAA | |
| site | YX788 | TGGAGTTCAGACGTGTGCTCTTCCGATCTCACTGTACTTG | 206 |
| 22_OT1_Rev | | CCCTGACCA | |
| site | YX789 | ACACTCTTTCCCTACACGACGCTCTTCCGATCTNNNNTTG | 207 |
| 22_OT2_Fwd | | GTGTTGACAGGGAGCAA | |
| site | YX790 | TGGAGTTCAGACGTGTGCTCTTCCGATCTCTGAGATGTGG | 208 |
| 22_OT2_Rev | | GCAGAAGGG | |
| site | YX791 | ACACTCTTTCCCTACACGACGCTCTTCCGATCTNNNNTGA | 209 |
| 22_OT3_Fwd | | GAGGGAACAGAAGGGCT | |
| site | YX792 | TGGAGTTCAGACGTGTGCTCTTCCGATCTGTCCAAAGGCC | 210 |
| 22_OT3_Rev | | CAAGAACCT | |
| site | YX563 | ACACTCTTTCCCTACACGACGCTCTTCCGATCTNNNNGGG | 211 |
| 23_OT1_Fwd | | AGATTTGCATCTGTGGAGG | |
| site | YX564 | TGGAGTTCAGACGTGTGCTCTTCCGATCTGCTTTTATACC | 212 |
| 23_OT1_Rev | | ATCTTGGGGTTACAG | |
| site | YX565 | ACACTCTTTCCCTACACGACGCTCTTCCGATCTNNNNCAA | 213 |
| 23_OT2_Fwd | | TGTGCTTCAACCCATCACGG | |
| site | YX566 | TGGAGTTCAGACGTGTGCTCTTCCGATCTCCATGAATTTG | 214 |
| 23_OT2_Rev | | TGATGGATGCAGTCTG | |
| site | YX943 | ACACTCTTTCCCTACACGACGCTCTTCCGATCTNNNNagga | 215 |
| 23_OT3_Fwd | | ggtgcaggagctagac | |
| site | YX944 | TGGAGTTCAGACGTGTGCTCTTCCGATCTtcctcgtcctgctctcactt | 216 |
| 23_OT3_Rev | | ag | |

| | | | SEQ | | Effector |
|---|---|---|---|---|---|
| Site | Plasmid | Spacer | ID NO: | PAM | protein |
| site 1 | 034c | GAACACAAAGCATAGACTGC | 217 | GGG | SpCas9 |
| site 2 | 034d | AAGTGTGATCACTTGGGTGG | 218 | TGG | SpCas9 |
| site 3 | 060e | CAGGACGGTCACCTTTGGGG | 219 | TGG | SpCas9 |
| site 4 | 122e | AGGATGCAAGTTTGTCTTGG | 220 | GGG | SpCas9 |
| site 5 | 060b | GGTACCTATCGATTGTCAGG | 221 | AGG | SpCas9 |
| site 6 | 034j | GAGTATGAGGCATAGACTGC | 222 | AGG | SpCas9 |
| site 7 | 034n | GGATTGACCCAGGCCAGGGC | 223 | TGG | SpCas9 |
| site 8 | 034r | GAAGACCAAGGATAGACTGC | 224 | TGG | SpCas9 |
| site 9 | 034v | AGCCAGGACGGTCACCTTTG | 225 | GGG | SpCas9 |
| site 10 | 034w | GACAAGTGTGATCACTTGGG | 226 | TGG | SpCas9 |
| site 11 | 034x | CCAAGGATTGACCCAGGCCA | 227 | GGG | SpCas9 |
| site 12 | 122b | CCAAGTACTTCAGGTAGCTG | 228 | AGG | SpCas9 |
| site 13 | 034m | GATGAGATAATGATGAGTCA | 229 | GGG | SpCas9 |
| site 14 | 120d | aagcaattgttatgattaaa | 230 | TGG | SpCas9 |
| site 15 | 120n | aatacattccaaaagaaatg | 231 | GGG | SpCas9 |
| site 16 | 120o | gaatacattccaaaagaaat | 232 | GGG | SpCas9 |
| site 17 | 120p | tgaatacattccaaaagaaa | 233 | TGG | SpCas9 |
| site 18 | 121f | ATATTTGCATTGAGATAGTG | 234 | TGG | SpCas9 |
| site 19 | 121g | CCACCGCTGCGCCAAGGTGC | 235 | GGG | SpCas9 |
| site 20 | 121j | TAAGGCCCAAGGGGGCAAGC | 236 | TGG | SpCas9 |
| site 21 | 121k | GCAGGTGACCGTGGCCTGCG | 237 | AGG | SpCas9 |
| site 22 | 034z | GGCCCAGACTGAGCACGTGA | 238 | TGG | SpCas9 |
| site 23 | 034y | GAGTCCGAGCAGAAGAAGAA | 239 | GGG | SpCas9 |
| R loop 1 | 069a | GTGGTAGACAGCATGTGTCCTA | 240 | AAGG | SaCas9 |
| | | | | GT | |
| R loop 2 | 069b | GATTTACAGCCTGGCCTTTGGGG | 241 | TCGG | SaCas9 |
| | | | | GT | |
| R loop 3 | 069c | GTGTCAGGTAATGTGCTAAACA | 242 | GAGA | SaCas9 |
| | | | | GT | |
| R loop 4 | 069d | GGTGGAGGAGGGTGCATGGGGT | 243 | CAGA | SaCas9 |
| | | | | AT | |
| R loop 5 | 069f | GGCAAGAGGATTGATTGAGCCA | 244 | GAGA | SaCas9 |
| | | | | GT | |
| R loop 6 | 069k | ACTAGTGTGCGAAGTATCATAA | 245 | AGGA | SaCas9 |
| | | | | GT | |
| site 24 | 119a | GGTTAACAAGGCCAAACTCC | 246 | AGA | NG/VRQR- |
| | | | | | SpCas9 |
| site 25 | 119b | GGGTCCAGTTCCGGGATTAG | 247 | CGA | NG/VRQR- |
| | | | | | SpCas9 |
| site 26 | 119k | CAAGGATAGACTGCTGGGCT | 248 | TGA | NG/VRQR- |
| | | | | | SpCas9 |
| site 27 | 119f | GAGGACAAAGUACAAACGGC | 249 | AGA | VRQR-SpCas9 |
| site 28 | 119d | GATGACCCGTATTATCTGGC | 250 | AGT | NG-SpCas9 |
| site 29 | 119i | GGAGACATCAAACCATGACT | 251 | TGC | NG-SpCas9 |
| site 30 | 128a | CAAGTGATCACACTTGTCAC | 252 | CACC | NRCH-SpCas9 |
| site 31 | 128b | ATAGACTGCAGGAGACATCA | 253 | AACC | NRCH-SpCas9 |
| site 32 | 129a | ATGACTTGCAGATGAAGAAG | 254 | CATT | NRTH-SpCas9 |
| site 33 | 129d | gattcaaagccatttttcca | 255 | GATA | NRTH-SpCas9 |
| site 34 | 069a | GTGGTAGACAGCATGTGTCCTA | 240 | AAGG | SaCas9 |
| | | | | GT | |
| site 35 | 069b | GATTTACAGCCTGGCCTTTGGGG | 241 | TCGG | SaCas9 |
| | | | | GT | |
| site 36 | 069c | GTGTCAGGTAATGTGCTAAACA | 242 | GAGA | SaCas9 |
| | | | | GT | |
| site 37 | 069d | GGTGGAGGAGGGTGCATGGGGT | 243 | CAGA | SaCas9 |
| | | | | AT | |
| site 38 | 069k | ACTAGTGTGCGAAGTATCATAA | 245 | AGGA | SaCas9 |
| | | | | GT | |
| site 39 | 069l | TACAGAGGGACAGAGGCCTGAC | 256 | CTGG | SaCas9 |
| | | | | GT | |
| site 40 | 115h | ATGAGAAGTATGACAACAGCCT | 257 | CAAG | SaKKH_ |
| | | | | AT | SaCas9 |
| site 41 | 115i | GGCAGTCATCTTAGTCATTACC | 258 | TGAG | SaKKH_ |
| | | | | GT | SaCas9 |
| site 42 | 115k | GGACTAGAGTAGGATTGTACCC | 259 | CTCA | SaKKH_ |
| | | | | GT | SaCas9 |
| site 43 | 115m | GGCTGAGCTAACTGTGACAGCA | 260 | TGTG | SaKKH_ |
| | | | | GT | SaCas9 |
| site 44 | 113a/ | TGCTGCAAGTAAGCATGCATTTG | 261 | TTTC | LbCpf1/ |
| | 114a | | | | enAsCpf1 |
| site 45 | 113b/ | CTAGACAGGGGCTAGTATGTGCA | 262 | TTTC | LbCpf1/ |
| | 114b | | | | enAsCpf1 |
| site 46 | 113c/ | CAGCTATTCAGGCTGGCCCGCCC | 263 | TTTG | LbCpf1/ |
| | 114c | | | | penAsCf1 |
| site 47 | 113d/ | GAAGCACATCAAGGACATTCTAA | 264 | TTTA | LbCpf1/ |
| | 114d | | | | penAsCf1 |
| site 48 | 113e/ | GGATAAGCACAGTTTTAAATAGT | 265 | TTTG | LbCpf1/ |
| | 114e | | | | penAsCf1 |
| site 49 | 113f/ | GTTTAAACACACCGGGTTAATAA | 266 | TTTG | LbCpf1/ |
| | 114f | | | | penAsCf1 |
| site | 121j | TAAGGCCCAAGGGGGCAAGC | 236 | TGG | SpCas9 |
| 50_PCSK9 | | | | | |
| site | 121k | GCAGGTGACCGTGGCCTGCG | 237 | AGG | SpCas9 |
| 51_PCSK9 | | | | | |
| site | 133d | TGTCGAAGTTCGCCCTGGAG | 267 | AGG | SpCas9 |
| 52_ABCA4 | | | | | |
| site | 133e | CGAAGTTCGCCCTGGAGAGG | 268 | TGG | SpCas9 |
| 53_ABCA4 | | | | | |
| plasmid | 001a | GCTCTG6mATCTGAATACCACG | 269 | AGG | SpCas9 |
| G6mATC | | | | | |
| site | | | | | |
| plasmid | 034d | AAGTGTGATCACTTGGGTGG | 218 | TGG | SpCas9 |
| GATC | | | | | |
| site | | | | | |
| site 1 | | gaacacaatgcatagattgc | 270 | CGG | SpCas9 |
| OT1 | | | | | |
| site 1 | | aaacataaagcatagactgc | 271 | AAA | SpCas9 |
| OT2 | | | | | |
| site 22 | | cacccagactgagcacgtgc | 272 | TGG | SpCas9 |
| OT1 | | | | | |
| site 22 | | gacacagaccgggcacgtga | 273 | GGG | SpCas9 |
| OT2 | | | | | |
| site 22 | | agctcagactgagcaagtga | 274 | GGG | SpCas9 |
| OT3 | | | | | |
| site 22 | | agaccagactgagcaagaga | 275 | GGG | SpCas9 |
| OT4 | | | | | |
| site 23 | | GAGTTAGAGCAGAAGAAGAA | 276 | AGG | SpCas9 |
| OT1 | | | | | |
| site 23 | | GAGTCTAAGCAGAAGAAGAA | 277 | GAG | SpCas9 |
| OT2 | | | | | |
| site 23 | | gaggccgagcagaagaaaga | 278 | CGG | SpCas9 |
| OT3 | | | | | |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the references cited throughout the disclosure, to the extent that they provide exemplary procedural or other details supplementary to those set forth herein, are specifically incorporated herein by reference.
1. Landrum, M. J. et al. ClinVar: public archive of relationships among sequence variation and human phenotype. Nucleic Acids Res. 42, D980-985 (2014).
2. Landrum, M. J. et al. ClinVar: public archive of interpretations of clinically relevant variants. Nucleic Acids Res. 44, D862-868 (2016).
3. Gaudelli, N. M. et al. Programmable base editing of A*T to G*C in genomic DNA without DNA cleavage. Nature 551, 464-471 (2017).
4. Rees, H. A. & Liu, D. R. Base editing: precision chemistry on the genome and transcriptome of living cells. Nat. Rev. Genet. 19, 770-788 (2018).
5. Wolf, J., Gerber, A. P. & Keller, W. tadA, an essential tRNA-specific adenosine deaminase from Escherichia coli. EMBO J. 21, 3841-3851 (2002).
6. Huang, T. P. et al. Circularly permuted and PAM-modified Cas9 variants broaden the targeting scope of base editors. Nat. Biotechnol. 37, 626-631 (2019).
7. Richter, M. F. et al. Phage-assisted evolution of an adenine base editor with improved Cas domain compatibility and activity. Nat. Biotechnol. 38, 883-891 (2020).
8. Anzalone, A. V., Koblan, L. W. & Liu, D. R. Genome editing with CRISPR-Cas nucleases, base editors, transposases and prime editors. Nat. Biotechnol. 38, 824-844 (2020).
9. Zeng, Y. et al. Correction of the Marfan Syndrome Pathogenic FBN1 Mutation by Base Editing in Human Cells and Heterozygous Embryos. Mol. Ther. 26, 2631-2637 (2018).
10. Ryu, S. M. et al. Adenine base editing in mouse embryos and an adult mouse model of Duchenne muscular dystrophy. Nat. Biotechnol. 36, 536-539 (2018).
11. Liu, Z. et al. Highly efficient RNA-guided base editing in rabbit. Nat. Commun. 9, 2717 (2018).
12. Song, C. Q. et al. Adenine base editing in an adult mouse model of tyrosinaemia. Nat. Biomed. Eng. 4, 125-130 (2020).
13. Li, C. et al. Expanded base editing in rice and wheat using a Cas9-adenosine deaminase fusion. Genome Biol. 19, 59 (2018).
14. Hua, K., Tao, X., Yuan, F., Wang, D. & Zhu, J. K. Precise A.T to G.C Base Editing in the Rice Genome. Mol. Plant 11, 627-630 (2018).
15. Yan, F. et al. Highly Efficient A.T to G.C Base Editing by Cas9n-Guided tRNA Adenosine Deaminase in Rice. Mol. Plant 11, 631-634 (2018).
16. Koblan, L. W. et al. In vivo base editing rescues Hutchinson-Gilford progeria syndrome in mice. Nature 589, 608-614 (2021).
17. Newby, G. A. et al. Base editing of haematopoietic stem cells rescues sickle cell disease in mice. Nature 595, 295-302 (2021).
18. Musunuru, K. et al. In vivo CRISPR base editing of PCSK9 durably lowers cholesterol in primates. Nature 593, 429-434 (2021).
19. Rothgangl, T. et al. In vivo adenine base editing of PCSK9 in macaques reduces LDL cholesterol levels. Nat. Biotechnol. 39, 949-957 (2021).
20. Zhang, W. et al. Multiplex precise base editing in cynomolgus monkeys. Nat. Commun. 11, 2325 (2020).
21. Koblan, L. W. et al. Improving cytidine and adenine base editors by expression optimization and ancestral reconstruction. Nat. Biotechnol. 36, 843-846 (2018).
22. Gaudelli, N. M. et al. Directed evolution of adenine base editors with increased activity and therapeutic application. Nat. Biotechnol. 38, 892-900 (2020).
23. Li, J. et al. Structure-guided engineering of adenine base editor with minimized RNA off-targeting activity. Nat. Commun. 12, 2287 (2021).
24. Kleinstiver, B. P. et al. Engineered CRISPR-Cas9 nucleases with altered PAM specificities. Nature 523, 481-485 (2015).
25. Nishimasu, H. et al. Engineered CRISPR-Cas9 nuclease with expanded targeting space. Science 361, 1259-1262 (2018).
26. Miller, S. M. et al. Continuous evolution of SpCas9 variants compatible with non-G PAMs. Nat. Biotechnol. 38, 471-481 (2020).
27. Ran, F. A. et al. In vivo genome editing using Staphylococcus aureus Cas9. Nature 520, 186-191 (2015).
28. Kleinstiver, B. P. et al. Broadening the targeting range of Staphylococcus aureus CRISPR-Cas9 by modifying PAM recognition. Nat. Biotechnol. 33, 1293-1298 (2015).
29. Zetsche, B. et al. Cpf1 is a single RNA-guided endonuclease of a class 2 CRISPR-Cas system. Cell 163, 759-771 (2015).
30. Kleinstiver, B. P. et al. Engineered CRISPR-Cas12a variants with increased activities and improved targeting ranges for gene, epigenetic and base editing. Nat. Biotechnol. 37, 276-282 (2019).
31. Rees, H. A., Wilson, C., Doman, J. L. & Liu, D. R. Analysis and minimization of cellular RNA editing by DNA adenine base editors. Sci. Adv. 5, eaax5717 (2019).
32. Fang, G. et al. Genome-wide mapping of methylated adenine residues in pathogenic Escherichia coli using single-molecule real-time sequencing. Nat. Biotechnol. 30, 1232-1239 (2012).
33. Marinus, M. G. & Lobner-Olesen, A. DNA Methylation. EcoSal Plus 6 (2014).
34. Losey, H. C., Ruthenburg, A. J. & Verdine, G. L. Crystal structure of Staphylococcus aureus tRNA adenosine deaminase TadA in complex with RNA. Nat. Struct. Mol. Biol. 13, 153-159 (2006).
35. Cadwell, R. C. & Joyce, G. F. Randomization of genes by PCR mutagenesis. PCR Methods Appl 2, 28-33 (1992).
36. Grunewald, J. et al. CRISPR DNA base editors with reduced RNA off-target and self-editing activities. Nat. Biotechnol. 37, 1041-1048 (2019).
37. Thuronyi, B. W. et al. Continuous evolution of base editors with expanded target compatibility and improved activity. Nat. Biotechnol. 37, 1070-1079 (2019).
38. Tsai, S. Q. et al. GUIDE-seq enables genome-wide profiling of off-target cleavage by CRISPR-Cas nucleases. Nat. Biotechnol. 33, 187-197 (2015).
39. Tsai, S. Q. et al. CIRCLE-seq: a highly sensitive in vitro screen for genome-wide CRISPR-Cas9 nuclease off-targets. Nat. Methods 14, 607-614 (2017).
40. Doman, J. L., Raguram, A., Newby, G. A. & Liu, D. R. Evaluation and minimization of Cas9-independent off-target DNA editing by cytosine base editors. Nat. Biotechnol. 38, 620-628 (2020).
41. Yu, Y. et al. Cytosine base editors with minimized unguided DNA and RNA off-target events and high on-target activity. Nat. Commun. 11, 2052 (2020).
42. Kleinstiver, B. P. et al. High-fidelity CRISPR-Cas9 nucleases with no detectable genome-wide off-target effects. Nature 529, 490-495 (2016).
43. Komor, A. C. et al. Improved base excision repair inhibition and bacteriophage Mu Gam protein yields C:G-to-T:A base editors with higher efficiency and product purity. Sci. Adv. 3, eaao4774 (2017).
44. Kim, Y. B. et al. Increasing the genome-targeting scope and precision of base editing with engineered Cas9-cytidine deaminase fusions. Nat. Biotechnol. 35, 371-376 (2017).
45. Li, X. et al. Base editing with a Cpf1-cytidine deaminase fusion. Nat. Biotechnol. 36, 324-327 (2018).
46. Park, S. W. et al. Post-transcriptional regulation of low density lipoprotein receptor protein by proprotein convertase subtilisin/kexin type 9a in mouse liver. J. Biol. Chem. 279, 50630-50638 (2004).
47. Musunuru, K. et al. In vivo CRISPR base editing of PCSK9 durably lowers cholesterol in primates. Nature 593, 429-434 (2021).
48. Rothgangl, T. et al. In vivo adenine base editing of PCSK9 in macaques reduces LDL cholesterol levels. Nat. Biotechnol. 39, 949-957 (2021).
49. Aguirre-Lamban, J. et al. Further associations between mutations and polymorphisms in the ABCA4 gene: clinical implication of allelic variants and their role as protector/risk factors. Invest Ophthalmol Vis Sci. 52, 6206-6212 (2011).

1. A polypeptide comprising SEQ ID NO:1, wherein the polypeptide comprises one or more amino acid substitutions relative to SEQ ID NO:1, wherein the one or more amino acid substitutions comprise a substitution at amino acid 23, 27, 36, 47, 48, 51, 76, 82, 106, 108, 109, 110, 111, 114, 119, 122, 123, 126, 127, 146, 147, 152, 154, 155, 156, 157, 161, 166, 167, and combinations thereof.

2. The polypeptide of claim 1, wherein the one or more amino acid substitutions comprise one or more of W23R, E27D, H36L, R47K, P48A, R51H, R51L, I76F, I76Y, V82S, A106V, D108G, A109S, K110R, T111H, A114V, D119N, H122R, H122N, H123Y, M126I, N127K, S146C, D147R, R152P, Q154R, E155V, I156F, K157N, K161N, T166I, and/or D167N,

3. The polypeptide of claim 1 or 2, wherein the polypeptide comprises a R47K substitution.

4. The polypeptide of any one of claims 1-4, wherein the polypeptide is not substituted at amino acid 84, 109, 122, 149, and/or 157.

5. The polypeptide of any one of claims 1-4, wherein the polypeptide comprises a D108G substitution.

6. The polypeptide of any one of claims 1-5, wherein the polypeptide comprises a K110R substitution.

7. The polypeptide of any one of claims 1-6, wherein the polypeptide comprises a T111H substitution.

8. The polypeptide of any one of claims 1-7, wherein the polypeptide comprises a T111R substitution.

9. The polypeptide of any one of claims 1-8, wherein the polypeptide comprises a A114V substitution.

10. The polypeptide of any one of claims 1-9, wherein the polypeptide comprises a M126I substitution.

11. The polypeptide of any one of claims 1-10, wherein the polypeptide comprises a N127K substitution.

12. The polypeptide of any one of claims 1-11, wherein the polypeptide comprises a W23R substitution.

13. The polypeptide of any one of claims 1-12, wherein the polypeptide comprises a E27D substitution.

14. The polypeptide of any one of claims 1-13, wherein the polypeptide comprises a H36L substitution.

15. The polypeptide of any one of claims 1-14, wherein the polypeptide comprises a P48A substitution.

16. The polypeptide of any one of claims 1-15, wherein the polypeptide comprises a R51H substitution.

17. The polypeptide of any one of claims 1-16, wherein the polypeptide comprises a R51L substitution.

18. The polypeptide of any one of claims 1-17, wherein the polypeptide comprises a I76F substitution.

19. The polypeptide of any one of claims 1-18, wherein the polypeptide comprises a I76Y substitution.

20. The polypeptide of any one of claims 1-19, wherein the polypeptide comprises a V82S substitution.

21. The polypeptide of any one of claims 1-20, wherein the polypeptide comprises a A106V substitution.

22. The polypeptide of any one of claims 1-21, wherein the polypeptide comprises a A109S substitution.

23. The polypeptide of any one of claims 1-22, wherein the polypeptide comprises a D119N substitution.

24. The polypeptide of any one of claims 1-23, wherein the polypeptide comprises a H122R substitution.

25. The polypeptide of any one of claims 1-24, wherein the polypeptide comprises a H122N substitution.

26. The polypeptide of any one of claims 1-25, wherein the polypeptide comprises a H123Y substitution.

27. The polypeptide of any one of claims 1-26, wherein the polypeptide comprises a M126I substitution.

28. The polypeptide of any one of claims 1-27, wherein the polypeptide comprises a S146C substitution.

29. The polypeptide of any one of claims 1-28, wherein the polypeptide comprises a D147R substitution.

30. The polypeptide of any one of claims 1-29, wherein the polypeptide comprises a R152P substitution.

31. The polypeptide of any one of claims 1-30, wherein the polypeptide comprises a Q154R substitution.

32. The polypeptide of any one of claims 1-31, wherein the polypeptide comprises a E155V substitution.

33. The polypeptide of any one of claims 1-32, wherein the polypeptide comprises a I156F substitution.

34. The polypeptide of any one of claims 1-33, wherein the polypeptide comprises a K157N substitution.

35. The polypeptide of any one of claims 1-34, wherein the polypeptide comprises a K161N substitution.

36. The polypeptide of any one of claims 1-35, wherein the polypeptide comprises a T166I substitution.

37. The polypeptide of any one of claims 1-36, wherein the polypeptide comprises a D167N substitution.

38. The polypeptide of any one of claims 1-37, wherein the one or more substitutions comprise or consist of D108G and K161N substitutions.

39. The polypeptide of any one of claims 1-38, wherein the one or more substitutions comprise or consist of P48A, D108G, and K161N substitutions.

40. The polypeptide of any one of claims 1-39, wherein the one or more substitutions comprise or consist of P48A, I76F, D108G, and K161N substitutions.

41. The polypeptide of any one of claims 1-40, wherein the one or more substitutions comprise or consist of P48A, R51H, 176F, D108G, K110R, H122R, M126I, N127K, and K161N substitutions.

42. The polypeptide of any one of claims 1-41, wherein the one or more substitutions comprise or consist of P48A, R51H, D108G, K110R, H122R, M126I, and N127K, substitutions.

43. The polypeptide of any one of claims 1-42, wherein the one or more substitutions comprise or consist of E27D, P48A, R51H, I76F, D108G, K110R, H122R, M126I, N127K, and K161N substitutions.

44. The polypeptide of any one of claims 1-43, wherein the one or more substitutions comprise or consist of E27D, R47K, P48A, R51H, I76F, D108G, K110R, H122R, M126I, N127K, and K161N substitutions.

45. The polypeptide of any one of claims 1-44, wherein the one or more substitutions comprise or consist of E27D, P48A, R51H, D108G, K110R, A114V, H122R, M126I, and N127K substitutions.

46. The polypeptide of any one of claims 1-45, wherein the one or more substitutions comprise or consist of E27D, R47K, P48A, R51H, I76F, D108G, K110R, A114V, H122R, M126I, and N127K substitutions.

47. The polypeptide of any one of claims 1-46, wherein the one or more substitutions comprise or consist of P48A, R51H, I76F, A106V, D108G, K110R, T111H, D119N, H123Y, M126I, N127K, D147R, R152P, Q154R, E155V, and I156F substitutions.

48. The polypeptide of any one of claims 1-47, wherein the one or more substitutions comprise or consist of P48A, R51H, I76F, A106V, D108G, K110R, T111H, D119N, H122R, H123Y, M126I, N127K, D147R, R152P, Q154R, E155V, and I156F substitutions.

49. The polypeptide of any one of claims 1-48, wherein the one or more substitutions comprise or consist of P48A, R51H, I76F, A106V, D108G, K110R, T111H, D119N, H123Y, M126I, N127K, S146C, D147R, R152P, Q154R, E155V, and I156F substitutions.

50. The polypeptide of any one of claims 1-49, wherein the one or more substitutions comprise or consist of P48A, R51H, I76F, A106V, D108G, K110R, T111H, D119N, H123Y, M126I, N127K, D147R, R152P, Q154R, E155V, I156F, and K157N substitutions.

51. The polypeptide of any one of claims 1-50, wherein the one or more substitutions comprise or consist of P48A, R51H, I76F, A106V, D108G, K110R, T111H, D119N, H123Y, M126I, N127K, D147R, R152P, Q154R, E155V, I156F, and K161N substitutions.

52. The polypeptide of any one of claims 1-51, wherein the one or more substitutions comprise or consist of P48A, R51H, I76F, A106V, D108G, K110R, T111H, D119N, H123Y, M126I, N127K, D147R, R152P, Q154R, E155V, I156F, and T166I substitutions.

53. The polypeptide of any one of claims 1-52, wherein the one or more substitutions comprise or consist of P48A, R51H, I76F, A106V, D108G, K110R, T111H, D119N, H123Y, M126I, N127K, D147R, R152P, Q154R, E155V, I156F, and D167N substitutions.

54. The polypeptide of any one of claims 1-53, wherein the one or more substitutions comprise or consist of W23R, H36L, R47K, P48A, R51L, 176F, V82S, A106V, D108G, K110R, T111H, D119N, H123Y, M126I, N127K, S146C, D147R, R152P, Q154R, E155V, and I156F substitutions.

55. The polypeptide of any one of claims 1-54, wherein the one or more substitutions comprise or consist of W23R, H36L, R47K, P48A, R51L, 176F, V82S, A106V, D108G, K110R, T111H, D119N, H122N, H123Y, M126I, N127K, S146C, D147R, R152P, Q154R, E155V, and I156F substitutions.

56. The polypeptide of any one of claims 1-55, wherein the one or more substitutions comprise or consist of W23R, H36L, R47K, P48A, R51L, I76Y, V82S, A106V, D108G, K110R, T111H, A114V, D119N, H123Y, M126I, N127K, S146C, D147R, R152P, Q154R, E155V, and I156F substitutions.

57. The polypeptide of any one of claims 1-56, wherein the one or more substitutions comprise or consist of W23R, H36L, R47K, P48A, R51L, 176F, V82S, A106V, D108G, A109S, K110R, T111H, A114V, D119N, H123Y, M126I, N127K, D147R, R152P, Q154R, E155V, and I156F substitutions.

58. The polypeptide of any one of claims 1-57, wherein the one or more substitutions comprise or consist of W23R, R47K, P48A, R51L, I76Y, V82S, A106V, D108G, A109S, K110R, T111H, A114V, D119N, H123Y, M126I, N127K, D147R, R152P, Q154R, E155V, and I156F substitutions.

59. The polypeptide of any one of claims 1-58, wherein the one or more substitutions comprise or consist of W23R, H36L, R47K, P48A, R51L, I76Y, V82S, A106V, D108G, K110R, T111H, A114V, D119N, H123Y, M126I, N127K, S146C, D147R, R152P, Q154R, E155V, I156F, K157N, K161N, T166I, and D167N substitutions.

60. The polypeptide of any one of claims 1-59, wherein the one or more substitutions comprise or consist of W23R, H36L, R47K, P48A, R51L, I76Y, V82S, A106V, D108G, K110R, T111H, A114V, D119N, H123Y, M126I, N127K, S146C, D147R, R152P, Q154R, E155V, I156F, T166I, and D167N substitutions.

61. The polypeptide of any one of claims 1-60, wherein the one or more substitutions comprise or consist of P48A, D108G, M126I, and K161N substitutions.

62. The polypeptide of any one of claims 1-61, wherein the one or more substitutions comprise or consist of P48A, D108G, N127K, and K161N substitutions.

63. The polypeptide of any one of claims 1-62, wherein the one or more substitutions comprise or consist of P48A, I76F, D108G, K110R, N127K, and K161N substitutions.

64. The polypeptide of any one of claims 1-57, wherein the one or more substitutions comprise or consist of P48A, R51H, 176F, D108G, K110R, M126I, N127K, and K161N substitutions.

65. The polypeptide of any one of claims 1-64, wherein the one or more substitutions comprise or consist of D108G, K110R, N127K, and K161N substitutions.

66. The polypeptide of any one of claims 1-65, wherein the one or more substitutions comprise or consist of P48A, R51H, I76F, A106V, D108G, K110R, T111H, D119N, H123Y, M126I, N127K, D147R, R152P, Q154R, E155V, and I156F substitutions.

67. The polypeptide of any one of claims 1-66, wherein the one or more substitutions comprise or consist of P48A, R51H, I76F, A106V, D108G, K110R, T111H, D119N, H123Y, M126I, N127K, S146C, D147R, R152P, Q154R, E155V, and I156F substitutions.

68. The polypeptide of any one of claims 1-67, wherein the one or more substitutions comprise or consist of P48A, R51H, I76F, A106V, D108G, K110R, T111H, D119N, H123Y, M126I, N127K, D147R, R152P, Q154R, E155V, I156F, and K157N substitutions.

69. The polypeptide of any one of claims 1-68, wherein the one or more substitutions comprise or consist of P48A, R51H, I76F, A106V, D108G, K110R, T111H, D119N, H123Y, M126I, N127K, D147R, R152P, Q154R, E155V, I156F, and K161N substitutions.

70. The polypeptide of any one of claims 1-69, wherein the one or more substitutions comprise or consist of P48A, R51H, I76F, A106V, D108G, K110R, T111H, D119N, H123Y, M126I, N127K, D147R, R152P, Q154R, E155V, I156F, and T166I substitutions.

71. The polypeptide of any one of claims 1-70, wherein the polypeptide comprises or consists of a polypeptide having the amino acid sequence of one of SEQ ID NOS:2-30 or 291-312.

72. The polypeptide of any one of claims 1-71, wherein the polypeptide comprises at least 75% sequence identity to SEQ ID NO:1.

73. The polypeptide of any one of claims 1-72, wherein the polypeptide comprises at least 75% sequence identity to one of SEQ ID NOS:2-30 or 291-312.

74. The polypeptide of claim 73, wherein the polypeptide comprises at least 80% sequence identity to SEQ ID NO:26.

75. The polypeptide of any one of claims 72-74, wherein the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, and/or 167 is substituted.

76. The polypeptide of claim 75, wherein the substitution is with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

77. The polypeptide of any one of claims 1-76, wherein the polypeptide comprises at least 2 amino acid substitutions relative to SEQ ID NO:1.

78. The polypeptide of claim 77, wherein the at least two substitutions are at amino acid positions 23, 27, 36, 47, 48, 51, 76, 82, 106, 108, 109, 110, 111, 114, 119, 122, 123, 126, 127, 146, 147, 152, 154, 155, 156, 157, 161, 166, and/or 167.

79. The polypeptide of claim 78, wherein the at least two substitutions are selected from W23R, E27D, H36L, R47K, P48A, R51H, R51L, I76F, I76Y, V82S, A106V, D108G, A109S, K110R, T111H, A114V, D119N, H122R, H122N, H123Y, M126I, N127K, S146C, D147R, R152P, Q154R, E155V, I156F, K157N, K161N, T166I, and D167N,

80. The polypeptide of any one of claims 1-79, wherein the polypeptide modifies adenosine bases in a nucleic acid molecule.

81. The polypeptide of claim 80, wherein the nucleic acid molecule is a RNA or a DNA molecule.

82. The polypeptide of claim 80 or 81, wherein the nucleic acid molecule is single-stranded.

83. The polypeptide of claim 80 or 81, wherein the nucleic acid molecule is double-stranded.

84. The polypeptide of any one of claims 1-82, wherein the polypeptide is covalently linked to an effector protein.

85. The polypeptide of claim 84, wherein the effector protein comprises a Cas protein, or a variant thereof.

86. The polypeptide of claim 85, wherein the effector comprises a catalytically impaired Cas protein.

87. The polypeptide of any one of claims 85-86, wherein the Cas protein comprises a Cas9 protein.

88. The polypeptide of claim 86 or 87, wherein the effector or Cas protein is further defined as Sp dCas9 (D10A, H840A), Sp nCas9 (D10A), Hf nCas9 (D10A), Sp VQR nCas9 (D10A), Sp VRER nCas9 (D10A), Sa nCas9 (D10A), Sa KKH nCas9 (D10A), dCas12a, SpCas9(D10A)-NG, xCas9 (D10A), Sp dCas9, Sp dCas9, Sp n xCas9 (D10A), Sa nCas9 (D10A), Sp Cas9-VRQR, SpCas9-NG, SpCas9-NRCH, SpCas9Nrth, LbCpf, enAsCpf, or SaKH nCas9 (D10A).

89. The polypeptide of any one of claims 84-88, wherein the effector protein comprises the amino acid sequence of one of SEQ ID NOS:281-290 or an amino acid sequence with at least 80% sequence identity to one of SEQ ID NOS:281-290.

90. The polypeptide of any one of claims 84-89, wherein the effector protein is fused to the N-terminus of the polypeptide.

91. The polypeptide of any one of claims 84-89, wherein the effector protein is fused to the C-terminus of the polypeptide.

92. The polypeptide of any one of claims 84-91, wherein the polypeptide comprises a linker between the effector protein and the polypeptide.

93. The polypeptide of claim 92, wherein the linker comprises SEQ ID NO:314 or an amino acid having at least 80% sequence identity to SEQ ID NO:314.

94. The polypeptide of any one of claims 1-93, wherein the polypeptide comprises one or more nuclear localization signals.

95. The polypeptide of any one of claims 1-94, wherein the polypeptide comprises SEQ ID NO:317 or an amino acid sequence having at least 85% sequence identity to SEQ ID NO:317.

96. A nucleic acid encoding the polypeptide of any one of claims 1-95.

97. An expression vector comprising the nucleic acid of claim 96.

98. A host cell comprising the polypeptide of any one of claims 1-95, the nucleic acid of claim 96, or the expression vector of claim 97.

99. A method of making a cell comprising transferring the nucleic acid of claim 96 or the expression vector of claim 97 into a cell.

100. A method for making a polypeptide comprising transferring the expression vector in claim 97 under conditions sufficient for expression of the polypeptide encoded on the expression vector.

101. A method for modifying adenine bases and/or for editing adenine bases in a nucleic acid molecule comprising contacting the nucleic acid with the polypeptide of any one of claims 1-95.

102. The method of claim 101, wherein the nucleic acid comprises DNA.

103. The method of claim 101, wherein the nucleic acid comprises RNA.

104. The method of any one of claims 101-103, wherein the nucleic acid comprises a Protospacer Adjacent Motif (PAM) motif and wherein the adenine is at a position at least 4, 5, 6, 7, 8, 9, 10, 11, 12, 13, 14, or 15 (or any derivable range therein) bases distal from the PAM.

105. The method of any one of claims 101-104, wherein the adenine is adjacent to a purine.

106. The method of any one of claims 101-104, wherein the adenine is adjacent to a pyrimidine.

107. The method of any one of claims 101-106, wherein the adenine base is modified to an inosine base.

108. The method of any one of claims 101-107, wherein the adenine base is edited to a guanine base.

109. The method of any one of claims 101-108, wherein the method is performed in vitro, in vivo, or ex vivo.

110. A method for directed evolution of an editor, the method comprising:
(i) generating a library of variant genes of the editor by mutagenesis;
(ii) selecting or screening for one or more variants with increased fitness, wherein each variant comprises one or more substitutions in the amino acid sequence of the editor;
(iii) generating a library of variant genes by mutagenesis, wherein the template variant genes comprises the one or more variants with increased fitness;
(iv) selecting or screening for one or more variants with increased fitness, wherein each variant comprises one or more substitutions in the amino acid sequence of the editor;
(v) repeating steps (iii) and (iv) iteratively between 0-10 additional times;
(vi) generating a library of variant genes; wherein the library comprises variant genes that combines the one or more substitutions of the selected variants of (iv) or (v);
(vii) selecting or screening for one or more variants with increased fitness; wherein each variant comprises one or more substitutions in the amino acid sequence of the editor;
(ix) repeating steps (iv) and (v) or steps (vii) and (viii) iteratively between 0-10 additional times.

111. The method of claim 110, wherein steps (i)-(ix) are performed in order.

112. The method of claim 110 or 111, wherein (i) generating a library of variant genes of the editor by mutagenesis comprises mutagenesis by chemical mutagens, error prone PCR, transposons, or DNA shuffling.

113. The method of claim 112, wherein the mutagenesis comprises mutagenesis by error prone PCR.

114. The method of any one of claims 110-113, wherein the library of comprises a combinatorial library with coverage of at least 80% coverage of the substitution combinations.

115. The method of any one of claims 110-114, wherein the library of comprises a combinatorial library with coverage of at least 95% coverage of the substitution combinations.

116. The method of claim 114 or 115, wherein the combinatorial library is created by overlapping PCR fragments comprising DNA encoding for the one or more substitutions.

117. The method of any one of claims 110-116, wherein the library comprises at least 1000 different editor variants.

118. The method of any one of claims 114-117, wherein the combinatorial library comprises combinations of at least 3 of the one or more substitutions.

119. The method of any one of claims 110-118, wherein steps (ii), (v), and/or (viii), comprise selecting for one or more variants with increased fitness, wherein the selection comprises editing of at least two different nucleotides of a selection gene.

120. The method of claim 119, wherein the selection gene comprises an antibiotic resistance gene.

121. The method of any one of claims 110-120, wherein the editor comprises TadA, Cas9, Cas11, Cas12, Cas13, a zinc finger, Cpf1, CDA1, ADAR, ADAR1, ADAR2, a deaminase, an adenine base editor, a cytidine deaminase, APOBEC1, first-generation base editor (BE1), BE2, BE3, HF-BE3, BE4-GAM, YE1-BE3, EE-BE3, YE2-BE3, VQR-BE3, VRER-BE3, Sa-BE3, Sa-BE4, SaBE4-Gam, SaKKH-BE3, Cas12a-BE, Target-AID, Target-AID-NG, xBE3, eA3A-BE3, A3A-BE3, BE-PLUS, TAM, CRISPR-X, ABE7.9, ABE7.10, xABE, ABESa, VQR-ABE, VRER-ABE, SaKKH-ABE, Gam, an editor of SEQ ID NO:1-33, or a substitutional variant thereof.

122. The method of any one of claims 110-121, wherein the increased fitness comprises an increase in the rate of deamination, increased editing of an adenine in a RA context, wherein R denotes a purine base and A denotes an adenine; increased editing of an adenine in a YA context, wherein Y denotes a pyrimidine base and A denotes an adenine; increased editing at protospacer positions 1, 2, and/or 3.

123. The method of any one of claims 110-122, wherein the method further comprises cloning and/or sequencing the variants with increased fitness.

124. The method of claim 123, wherein the variants are sequenced by Next generation sequencing methods.
